SEQUENCE LISTING (11 sequences)

SEQ ID NO: 1 - RNA, 23 nt, Human coronavirus
aauaguuuaaaaauuacagaaga

SEQ ID NO: 2 - RNA, 21 nt, Human coronavirus
uccuucuuuagaaacuauaca

SEQ ID NO: 3 - RNA, 24 nt, Human coronavirus
augucaucccuacuauaacucaaa

SEQ ID NO: 4 - RNA, 21 nt, Human coronavirus
uuaaaauauaaugaaaaugga

SEQ ID NO: 5 - RNA, 25 nt, Human coronavirus
cuugaagccccuuuucucuaucuuu

SEQ ID NO: 6 - RNA, 21 nt, Human coronavirus
caacuauaaauuaaacacaga

SEQ ID NO: 7 - RNA, 24 nt, Human coronavirus
uugaauacaccaaaagaucacauu

SEQ ID NO: 8 - DNA, 36 bp, Artificial Sequence
  Synthetic
gttgacattgattattgactagttattaatagtaat

SEQ ID NO: 9 - DNA, 18 bp, Artificial Sequence
  Synthetic
ccatagagcccaccgcat

SEQ ID NO: 10 - DNA, 30 bp, Artificial Sequence
  Synthetic
tagttattaatagtaatcaattacggggtc

SEQ ID NO: 11 - DNA, 26 bp, Artificial Sequence
  Synthetic
gcagtgaaaaaaatgctttatttgtg

(19) United States
(12) Patent Application Publication
(10) Pub. No.: US 2023/0398220 A1
(43) Pub. Date: Dec. 14, 2023

(54) SIRNA-NANOBOWL-MEDIATED INTERVENTION OF COVID-19

(71) Applicant: THE REGENTS OF THE UNIVERSITY OF CALIFORNIA, Oakland, CA (US)

(72) Inventors: Deependra Kumar Ban, San Diego, CA (US); Ratneshwar Lal, La Jolla, CA (US)

(21) Appl. No.: 18/251,227

(22) PCT Filed: Nov. 5, 2021
(86) PCT No.: PCT/US2021/058353

§ 371 Date: Apr. 28, 2023

Related U.S. Application Data
(60) Provisional application No. 63/110,931, filed on Nov. 6, 2020.

(51) Int. Cl. A61K 41/00; A61K 47/69; C12N 15/113; A61K 45/06; A61K 31/573; A61K 39/215; A61K 47/02

(52) U.S. Cl. CPC A61K 41/00; A61K 47/6929; C12N 15/1131; A61K 45/06; A61K 31/573; A61K 39/215; A61K 47/02; C12N 2310/14

(57) ABSTRACT

Provided are methods, systems, and devices that pertain to siRNA-based nanobowl-mediated intervention for COV-ID-19.

### CROSS-REFERENCE TO RELATED APPLICATIONS

This application claims the benefit of U.S. Provisional Patent Application No. 63/110,931 filed on Nov. 6, 2020, the contents of which are incorporated by reference in their entirety.

### STATEMENT REGARDING SPONSORED RESEARCH

This invention was made with government support under R01 AG028709 awarded by the National Institutes of Health. The government has certain rights in the invention.

### SEQUENCE LISTING

This application contains a Sequence Listing, which is submitted in ASCII format via EFS-Web, and is hereby incorporated by reference in its entirety. The ASCII copy, created on Nov. 5, 2021, is named SequenceListing.txt and is 8 KB in size.

### TECHNICAL FIELD

The present technology relates to methods, systems, and devices that pertain to small interfering RNA (siRNA)-based nanobowl-mediated intervention of SARS-CoV-2 replication and treatment for COVID-19.

### BACKGROUND

Coronaviruses are a large family of RNA viruses that cause diseases in mammals and birds. There are hundreds of different strains of coronaviruses, most of which circulate among animals such as pigs, camels, bats, and cats. Coronaviruses can infect humans and cause upper-respiratory tract diseases that range from mild to lethal in severity. Notably, new coronaviruses have emerged from animal reservoirs over the past two decades causing serious and widespread illnesses in humans, some of which have resulted in death.

In November 2002, SARS coronavirus (SARS-CoV) began infecting humans resulting in severe acute respiratory syndrome (SARS). The SARS epidemic affected 26 countries and resulted in more than 8000 cases by 2003. In September 2012, Middle East respiratory syndrome (MERS) was identified in humans and was caused by the MERS coronavirus (MERS-CoV). By late 2019, a total of 2494 laboratory-confirmed cases of MERS had been reported globally with 858 associated deaths and a fatality rate of 34.4%. To this day, MERS continues to cause sporadic and localized outbreaks.

The most recent novel coronavirus to cause worldwide pandemic and health crises is SARS-CoV-2, which causes coronavirus disease 2019 (COVID-19). SARS-CoV-2 emerged in December 2019 and was declared a global pandemic by the World Health Organization (WHO) on Mar. 11, 2020. According to recent reports, COVID-19 is highly contagious (>243 million positive cases to date) and causes a high morbidity rate (>4 million deaths to date) worldwide. COVID-19 symptoms include fever, cough, shortness of breath, myalgia, fatigue, pharyngitis, headache, hemoptysis, and gastrointestinal maladies. While SARS-CoV-2 infection does not necessarily result in COVID-19 or other symptoms, those who do develop COVID-19 or show symptoms can rapidly progress to severe disease or death. Those at greatest risk for developing COVID-19 are over the age of 65 or have comorbidities, such as cardiovascular disease, cancer, and other diseases and/or conditions that render humans more likely to develop an infection.

The SARS-CoV-2 pandemic is devastating to global economic and societal health and survival, yet there is no effective treatment available. Current attempts at finding effective treatment include WHO-recommended repurposing of already available medicines (e.g., remdesivir, lopinaovir/ritonavir, anti-inflammatory steroids) for treating COVID-19 infections. However, most of these drugs either fail to efficiently mitigate infections or have side effects. There are also efforts to develop vaccines via virus attenuation, using virus-specific proteins, and nucleic acids. But this approach will protect only people who are already immunized—not those with new infections. Additionally, siRNA-based approaches to block viral replication and the use of repurposed drugs would require a focused, on-demand, and image-guided (i.e., trackable) delivery system to ensure locally effective doses and to minimize systemic distribution of untoward effects. Therefore, a need exists for improved therapeutic strategies to inhibit viral spread, treat current COVID-19 infections, and prevent new infections.

### SUMMARY

The present technology relates to methods, systems, and devices of siRNA-based nanobowl-mediated intervention for treatment of viral infections and diseases, including those affecting the respiratory systems, for example, COVID-19.

In some aspects, provided are nanobowl-based therapeutic systems comprising a nanobowl and one or more nucleic acids targeting a virus. In some embodiments, the virus is a coronavirus, for example, SARS-CoV, MERS-CoV, SARS-CoV-2, or a variant thereof. In some embodiments, the one or more nucleic acids are conjugated to the nanobowl through disulfide bonds.

In some embodiments, the one or more nucleic acids comprise siRNAs. In some embodiments, siRNAs each comprise a nucleotide sequence that is identical or complementary to a genetic sequence of SARS-CoV-2. In some embodiments, the genetic sequence is conserved among different strains of SARS-CoV-2. In some embodiments, the genetic sequence is located in the Orf1ab, S, M, or N gene regions. In some embodiments, the siRNAs each comprise a nucleotide sequence that is at least 80%, at least 85%, at least 90%, at least 95%, at least 96%, at least 97%, at least 98%, at least 99%, or 100% identical to any one of SEQ ID NOs: 1-7. In some embodiments, the siRNAs each comprise a nucleotide sequence complementary to a nucleotide sequence that is at least 80%, at least 85%, at least 90%, at least 95%, at least 96%, at least 97%, at least 98%, at least 99%, or 100% identical to any one of SEQ ID NOs: 1-7.

In some embodiments, the nanobowl further comprises iron oxide (10) nanoparticles. In some embodiments, the nanobowl is coated with a heat-sensitive coating, a biodegradable coating, and/or a lipid coating.

In some embodiments, the therapeutic system further comprises one or more additional therapeutic agents loaded to the nanobowl, wherein the one or more additional therapeutic agents are selected from the group consisting of an antiviral agent, an anti-inflammatory agent, an antimalaria agent, and a biological agent. In some embodiments, the antiviral agent is selected from the group consisting of remdesivir, favipiravir, lopinavir/ritonavir, nitazoxanide, danoprevir, umifenovir, nafamostat, brequinar, merimepodib, molnupiravir, opaganib, and ivermectin. In some embodiments, the anti-inflammatory agent is selected from the group consisting of ruxolitinib, baricitinib, dapagliflozin, eicosapentaenoic acid (EPA), tocilizumab, sarilumab, ravulizumab, losmapimod, pacritinib, bucillamine, tradipitant, lenzilumab, acalabrutinib, otilimab, abivertinib maleate, selinexor, brequinar, ibudilast, apilimod dimesylate, gimsilumab, dociparastat sodium, itolizumab, pemziviptadil, prednisolone, dexamethasone, reparixin, brensocatib, emapalumab, and anakinra. In some embodiments, the antimalaria agent is hydroxychloroquine or chloroquine. In some embodiments, the biologic agent is an antibody specific to SARS-CoV-2 or a vaccine against SARS-CoV-2.

In some aspects, provided are compositions comprising the therapeutic system according to various embodiments of the present technology.

In some aspects, provided are methods of treating or preventing infections or diseases caused by a virus in a subject in need thereof, comprising administering to the subject a therapeutically effective amount of the therapeutic system or the composition according to various embodiments of the present technology. In some embodiments, the virus is a coronavirus, for example, SARS-CoV, MERS-CoV, SARS-CoV-2, or a variant thereof.

In some embodiments, the method further comprises delivering the therapeutic system or the composition to a target cell, tissue, or organ in the subject by application of an external stimulus. In some embodiments, the external stimulus comprises a magnetic field.

In some embodiments, the method further comprises controlling load release of the therapeutic system by application of an internal or external stimulus. In some embodiments, the internal stimulus comprises a biochemical substance. In some embodiments, the external stimulus comprises a magnetic field, light, heat, or pH.

### BRIEF DESCRIPTION OF THE DRAWINGS

FIG. 1A is a schematic of nanobowl functionalization and conjugation of siRNAs with a nanobowl encapsulated by target-specific molecules. FIG. 1B is a schematic of targeted, controlled delivery of COVID-19 drugs (e.g., monoclonal antibodies, mesylates, lopinavir/ritonavir, hydroxychloroquine, remdesivir) by external magnetic field and SARS-CoV-2 specific siRNA release in infected cells by glutathione activity.

FIGS. 2A-2B are schematics of siRNA-nanobowl-based intervention of COVID-19 replication.

FIGS. 3A-3L show development of nanobowls for cDNA transfection. FIG. 3A is a schematic of nanobowl synthesis and surface functionalization. A 100 nm polystyrene (PS) template was used to generate an eccentric cavity in the nanobowl synthesis from tetraethyl orthosilicate (TEOS) condensation. Dimethyl formamide (DMF) was used to dissolve away the PS template, followed by amine functionalization with 3-aminopropyltriethoxysilane (APTES). FIG. 3B shows generation of vector-less linearized cDNA construct by polymerase chain reaction (PCR) containing the promoter and poly A tail regions. FIG. 3C is a cDNA loading curve showing μg of cDNA bound per mg of amine-functionalized nanobowl (left y-axis, black) and percent cDNA loading efficiency calculated as percent of mixed cDNA that bound (right y-axis, red). Circles and squares depict data from supercoiled cDNA and linearized cDNA, respectively. The points depict ±scanning electron microscopy (SEM) from mean values, performed in triplicate. FIG. 3D shows unstained transmission electron microscopy (TEM) image of purified nanobowls. FIGS. 3E-3L are TEM images of acutely dissociated dorsal root ganglion (DRG) neurons (FIGS. 3E, 3G), SCG neurons (FIG. 3H), human embryonic kidney (HEK) cells (FIGS. 3F, 3J), ND7/23 (FIG. 3I), HeLa (FIG. 3K) and L-cells (FIG. 3L) following a 4-hour incubation with nanobowls (30 μg/ml). The TEM images (FIGS. 3E-3L) were taken from 60 nm thin sections with negative staining. Note that images in FIG. 3E and FIG. 3F show nanobowls at the point of internalization.

FIGS. 4A-4I show characterization of nanobowl size distribution. SEM (FIGS. 4A-4F) and TEM images (FIGS. 4G-4H) of DMF-washed, PS core removed and purified silica nanobowls. FIG. 4I shows dynamic light scattering (DLS) raw intensity data showing a representative nanobowl hydrodynamic size distribution in water before and after APTES (amine) functionalization.

FIGS. 5A-5C show thermogravimetric analysis (TGA) of nanobowl and amine-functionalized nanobowl. FIG. 5A shows total weight loss profile from 100-1000° C. of nanobowl (black) and amine-functionalized nanobowls (red). Individual weight loss (black) and differential of weight loss (red) are plotted with respect to temperature for nanobowls post-DMF wash (FIG. 5B) and nanobowls coated with APTES post-DMF wash (FIG. 5C). Peaks (local minima) in the differential graphs indicate major regions of weight loss. Below 100° C., weight loss in both samples is caused by loss of adsorbed water. Between 100-300° C., weight loss is caused by loss of bound water and solvents like ethanol that are used in the synthesis and purification of nanobowls. These two mass losses are similar between both samples (FIGS. 5B-5C). Above 300° C., the mass loss is higher in the amine-functionalized nanobowls due to presence of bound amines due to APTES salinization that burns off and causes weight loss between 300° C. to 1000° C. Weight loss values in different temperature regimes are presented in Table 2.

FIGS. 6A-6B show analysis of linearized and supercoiled DNA loading on a nanobowl. As shown in FIG. 6A, binding data for linear (Lin) and supercoiled (SC) clover were plotted and fitted. SC can be easily fit with an exponential increase model reaching a saturation plateau in the entire range from 0-50 μg/mg DNA dosage (red circles and black dotted line). However, linearized DNA binding on a nanobowl does not follow the same pattern of saturating binding as supercoiled. We were able to fit linear loading data in the range <=25 μg/mg (black squares and solid black line). In this range, the binding pattern follows an increasing exponential form, which is consistent between both types of DNA constructs. Y-axis was normalized to saturation binding value of supercoiled at 50 μg/ml and binding value of linearized at 25 μg/ml within a scale of 0 to 1. FIG. 6B shows amount of tdTomato (tdT) (linearized) cDNA-loaded (black) and loading efficiency (red) at various amounts mixed with nanobowls (0-50 μg/mg). Red and black traces show representative loading profiles, with mean and ±SEM values, performed in duplicate.

FIGS. 7A-7D show transfection of HEK cells with DOPE/DOTAP encapsulated Stöber silica nanoparticles. SEM (FIG. 7A) and DLS (FIG. 7B) characterization of Stöber silica nanoparticles after synthesis and purification were shown. DLS data was acquired from ˜50 μg/ml silica nanoparticles dispersed in water. DH=415.3±21.9 nm PDI 0.207±0.043 were measured. FIGS. 7C-7D show fluorescent microscopy images of 0.5 mg/ml clover cDNA-loaded Stöber silica nanobowls (˜9 μg clover supercoiled plasm id per mg silica nanoparticle) 48 hours post-transfection in HEK 298 cells captured with a 10× objective. FIG. 7C shows the fluorescence channel after pseudo-coloring and FIG. 7D shows the overlay of the fluorescence and the phase channels for the same field of view.

FIGS. 8A-8J show that encapsulation of nanobowls with “helper” lipids results in clover expression. FIGS. 8A-8B show Western blots of clover expression in HEK and ND7/23 cells transfected with nanobowl loaded with supercoiled clover cDNA without (FIG. 8A) and with (FIG. 8B) 1:1 DOPE:DOTAP coating. In FIG. 8A, each lane was loaded with 0.2 μg/μl; in FIG. 8B, 0.075 and 0.25 μg/μl per lane were loaded for HEK and ND7/23 cells, respectively. The 36 kDa band represents clover. Vinculin (116 kDa) was used as the loading control. FIG. 8C is a schematic drawing showing 100 nm extruded lipids were mixed with DNA-loaded nanobowls to prepare lipid-encapsulated nanobowls (LNBs). FIG. 8D shows TEM images of LNBs with negative staining showing a lipid layer (approx. 5 nm diameter) surrounding LNBs. The white arrow indicates the thickness of the lipid layer on the nanobowl. FIGS. 8E-8F show TEM images of LNBs internalized in HEK (FIG. 8E) & ND7/23 (FIG. 8F) cells 4 hours after treatment with 0.5 mg/ml. The white arrows indicate nanobowl clusters found in the cytoplasm. FIGS. 8G-8J show phase (left), fluorescence (middle), and overlay (right) images acquired with 20×objective showing clover expression in transfected cells with linearized and supercoiled clover at 10 μg/mg LNB (0.5 mg/ml) after 48 hours. All scale bars measure 50 μm.

FIGS. 9A-9F show determination of LNB toxicity. FIGS. 9A-9D show viability (% live cells) measurement with the MTT assay of cells incubated in LNBs (0-1 mg/ml). The summary plots depict mean±SEM, performed in triplicate. The 0 mg/ml data point refers to cells treated only with 1:1 Opti-Minimum Essential Media (MEM) and DPBS and not exposed to nanobowls. FIG. 9E shows flow cytometry scatter plots showing single HEK cell populations with respect to clover emission (y-axis) and live/dead dye 7-AAD emission (x-axis). Quadrant Q1 (I) shows live cells expressing clover (9.1%), Q2 (II) shows dead cells expressing clover (1%), Q3 (III) shows live cells with no/negligible clover expression (82.8%), and Q4 (IV) shows dead cells with no/negligible clover expression (7.1%). This scatter plot was collected from HEK cells treated with 0.5 mg/ml nanobowls loaded with 10 μg/mg linearized clover cDNA for 48 hours. FIG. 9F shows plot of HEK cell viability (red) and clover expression (black) 48 hours post-transfection with varying LNB concentrations (0.05-1.0 mg/ml) loaded with 10 μg/mg linearized clover cDNA.

FIGS. 10A-10F show dose response of clover expression in HEK and ND7/23 cells. FIGS. 10A, 10B, 10D, 10E show Western blot experiments illustrating clover expression in HEK cells and ND7/23 cells 48 hours post-transfection with either supercoiled or linear cDNA under varying μg/mg cDNA loading. The cells were transfected with LNBs (0.5 mg/ml) loaded with increasing amounts of cDNA (0-50 μg/mg). The Western blots used anti-green fluorescent protein (GFP) and anti-vinculin (loading control). The 36 kDa band represents clover expressed in each sample. FIGS. 10C, 10F show the densitometric analysis of the Western blots for relative clover and vinculin expression. The values represent the mean with standard deviation of 2 independent experiments for supercoiled cDNA (FIG. 10C) and 2 independent measurements of a representative experiment for linearized cDNA (FIG. 10F).

FIGS. 11A-11F show transfection of DRG neurons with LNBs. FIGS. 11A-11B show TEM images of acutely dissociated DRG neurons acquired 4 (FIG. 11A) and 24 (FIG. 11B) hours post-treatment with LNBs (0.5 mg/ml). The images were taken from negatively stained 60 nm tissue sections. Clover expression in dissociated DRG neurons (FIG. 11C) and glial cells (FIG. 11D) 48 hours post-transfection (in vitro) with clover cDNA-loaded (10 μg/mg) LNBs (0.5 mg/ml) are shown. Phase (i) and fluorescence images (ii) were taken with a 40× objective. Scale bars in FIGS. 11C-11D depict 50 μm. FIGS. 11E-11F show phase (i) and fluorescence (ii) images of acutely dissociated DRG neurons taken 72 hours post-transfection (ex vivo) of DRG tissue with LNBs (1 mg/ml) loaded with tdT (50 μg/mg). The images were acquired with a 20×objective. Scale bars in FIGS. 11E-11F depict 50 μm.

FIG. 12 shows LNB dose-dependent transfection of HEK cells with linearized clover cDNA. Phase (left), fluorescence (middle), and overlay (right) images acquired with a 10× objective of HEK cells 48 hours post-transfection with varying concentrations of LNB (0.05-1 mg/ml) loaded with 10 μg/mg linearized clover cDNA. The samples were analyzed in flow cytometry for viability and toxicity trends (FIGS. 9E-9F). Bottom row represents positive control used in flow cytometry with Lipofectamine 2000 (4 μg) 24 hours post-transfection. All scale bars measure 100 μm.

FIGS. 13A-13B show types of linear DNA chemisorption schemes used with nanobowls. FIG. 13A shows that amine group on the nanobowl was covalently linked to carboxyl-terminated linearized DNA with an EDC linker (EDC conjugation chemistry). FIG. 13B shows that amine-coated nanobowls were first conjugated with DBCO N-hydroxysuccinimide (NHS) ester to confer DBCO functionality on nanobowls followed by attachment of azido-terminated linearized cDNA on DBCO-functionalized nanobowls by click chemistry.

FIGS. 14A-14G show transfection of HEK and ND7/23 cells with functionalized linear cDNA-loaded LNBs. FIG. 14A shows a 48-hour release profile of carboxylated (Lin-C) and azido-functionalized (Lin-A) linearized cDNA from nanobowl surfaces in DPBS, with or without p-mercaptoethanol at 37° C. FIG. 14B shows a Western blotting plot illustrating clover expression (36 kDa) in HEK cells 48 hours post-transfection with 0.5 mg/ml LNB loaded with Lin-A, Lin-C, linear (Lin), and supercoiled (SC) cDNAat 10 μg/mg LNB. Vinculin was used as the loading control (116 kDa). FIG. 14C shows mean (±standard deviation) relative expression levels of clover and vinculin in HEK cells when transfected with either linear or supercoiled cDNA constructs bound to LNBs. FIGS. 14D-14G show phase (left), fluorescence (center), and overlay (right) images acquired with a 20×objective showing clover expression in HEK cells and ND7/23 cells following transfection with LNB loaded (0.5 mg/ml) with Lin-C (FIGS. 14D, 14F) or Lin-A (FIGS. 14E, 14G) cDNA (10 μg/mg). Scale bars indicate 50 μm.

FIGS. 15A-15D show transfection of HeLa cells with LNBs carrying 4 types of clover cDNA constructs. HeLa cells were transfected for 4 hours with 0.5 mg/ml LNB loaded with either supercoiled (SC) or linearized (Lin) or carboxylated linearized (Lin-C) or azido linearized (Lin-A) clover cDNA (10 μg/mg). Phase (left), fluorescence images (middle), and overlay (right) were acquired with a 20× objective 48 hours post-transfection. Scale bars indicate 50 μm.

FIGS. 16A-16D show transfection of L-cells with LNBs carrying 4 types of clover cDNA constructs. L-cells were transfected for 4 hours with 0.5 mg/ml LNB loaded with either SC or Lin or Lin-C or Lin-A clover cDNA (10 μg/mg). Phase (left), fluorescence (middle), and overlay (right) images were acquired with a 20× objective 48 hours post-transfection. Scale bars indicate 50 μm.

FIGS. 17A-17G show coupling of GIRK channels with YFP-tagged MOR or kappa opioid receptor (KOR) in HEK cells. FIG. 17A shows phase contrast (i) and fluorescence (ii) images of HEK cells transfected with LNBs (0.5 mg/ml) loaded with YFP-MOR, GIRK1, and GIRK4 cDNA plasmids. Images were acquired with a 20×objective (scale bar 50 μm). Inset in (ii) depicts a fluorescence image acquired with a 40×objective (scale bar 50 μm). FIG. 17B shows raw traces of fluorescence signals before and during MOR agonist Oxycodone application (dotted line at t=30 second) in HEK cells transfected with GIRK1, GIRK4, and YFP-MOR Y-axis depicts the percentage change in relative fluorescence units (RFU). FIG. 17C shows concentration-response relationship of Oxycodone applied to MOR transfected HEK cells. FIG. 17D shows raw traces of fluorescence signals during application of Fentanyl to MOR transfected HEK cells. FIG. 17E shows raw traces of fluorescence signals before and during agonist application (dotted line at t=30 second) in HEK cells transfected with GIRK1, GIRK4, and K opioid receptors (KORs) at various doses of agonist U-50488. FIG. 17F shows concentration-response of U-50488 applied to KOR transfected HEK cells. FIG. 17G shows raw fluorescence traces for KOR opioid U-69593. Each point in (FIG. 17C) and (FIG. 17F) represents the mean percentage change of the RFU. The smooth curves were obtained by fitting the points to the Hill equation.

FIGS. 18A-18B show internalization of Cy3-tagged LNBs in DRG tissue incubated 6 hours in 0.5 or 1 mg/ml Cy3-LNBs. The DRG tissue was enzymatically dissociated 6 hours post-transfection period. Phase (i) and fluorescence images (ii) were acquired with a 20× objective. White arrows indicate dissociated neurons. Scale bars indicate 50 μm.

FIG. 19 is a schematic showing the design of a lipid-encapsulated, DNA-loaded nanobowl and the uptake of the nanobowl into a cell through endosomal entrapment and release for expression of proteins encoded by the DNA.

FIG. 20A is a schematic of nanobowl functionalization showing conjugation of siRNA with nanobowl functionalized with S-protein. FIG. 20B is a schematic of targeted delivery of S-protein functionalized nanobowl, controlled payload delivery of monoclonal antibody (mAb), repurposed drug (e.g., Remdesivir/Lopinavir), and anti-inflammatory drugs by external magnetic field, and SARS-CoV-2 specific siRNA release in infected cells by glutathione activity. The bottom panel shows SEM and fluorescence images of primary DRG neurons showing internalization of nanobowl and nucleic acid.

FIG. 21 shows schematic and electron microscopy (EM) images of various morphologies a nanobowl can adapt.

FIG. 22 shows design of a nanocarrier with enhanced on/off release capability (left) and magnetic guided delivery of theragnostic carriers to defined sites (right).

FIGS. 23A-23D show internalization and transfection of rat DRG neurons with nanobowls. FIG. 23A shows TEM images showing internalization of lipid-coated nanobowls in neurons from acutely dissociated rat DRG. FIG. 23B shows phase, fluorescence, and overlay images taken with 20× objective showing expression of linearized tdT cDNA in dissociated neurons from nanobowl transfected DRG tissues. FIG. 23C shows phase, fluorescence, and overlay images taken with 20× objective showing expression of linearized tdT cDNA in glia dissociated from nanobowl-transfected DRG tissues. FIG. 23D shows phase, fluorescence, and overlay images of an HEK cell line transfected with linearized clover cDNA. All optical microscopy scale bars are 50 μm.

FIG. 24A shows in vivo mouse tumor penetration using a Sm—Co magnet over the skin near the induced mouse tumor (for 2 hours) after the magnetic nanobowls (MNBs) were tail injected into the blood stream. The red spot on the mice represents the tumor site. Average number of MNBs trapped in the tumor cells was ˜2 orders of magnitude greater with magnetic attraction vs the control sample (MNBs injected without magnetic field). FIG. 24B shows fluorescein (FITC) imaging in surgically obtained tumor tissues showing the accumulated MNBS in the tumor tissue using a magnet. The DAPI image shows the tumor structure. FIG. 24C shows y-z vertical section (left) and x-z horizontal section (right) of the tumor colony after 2 hours vertical magnetic force pull by a Sm—Co magnet placed under the glass slide supporting the colony. The white arrows indicate that after 2 hours, the control sample (with the drug capsules but no magnetic pull) contains no MNBs within the colony, while the magnetic vectored MNBs (green) are pulled toward the colony bottom area, passing through the tumor colony thickness completely (˜several cell thickness) (scale bar=50 μm). Actin for red, DNA (Dapi) for blue, and FITC marker for green. FIG. 24D shows comparative growth rates of MT2 (breast cancer cells), without vs with radio frequency (RF) drug release.

FIGS. 25A-25E show that magnetic force allows blood brain barrier (BBB) crossing of magnetic nanobowls (MNBs). A small magnet was implanted in the right hemisphere (FIG. 25B, right). One week after implantation, MNBs were injected by intravenous (i.v.) in the tail. FIG. 25A shows confocal analysis shows MNBs (green) in the ipsilateral hemisphere, with low background level in contralateral side; nuclei (red, TOPRO-3); scale bar: 500 μm. FIG. 25B, left, shows confocal images of coronal brain sections showing enrichment of the MNBs near where the magnet was placed. Scale bar: 100 μm. FIG. 25C shows external magnetic force increases MNB level in the brain. FIG. 25D shows relative fluorescence level from A (*p<0.05). FIG. 25E shows hematoxylin and eosin and confocal microscopy (left), perivascular and brain cortex accumulation of MNB (middle), and AFM image of MNB uptake in human endothelial cells (right).

FIG. 26 shows Cy3-loaded nanobowls internalized in acutely dissociated neurons post in vivo injection in rat DRG.

FIGS. 27A-27D show alternating magnetic field (AMF) actuation for diseased tissue-specific therapeutic delivery. FIG. 27A shows bulk temperature increases upon application of AMF to a solution of magnetic nanobowl (2 mg/ml), iron oxide (10) (Fe₃O₄) amounting to 0.06 mg/ml, with a schematic of setup on the left. FIG. 27B shows guiding efficiency for nanobowls was determined in vitro using particle trajectories. Nanobowl cluster trajectories were imaged in different fluid flow and magnetic conditions. V/B used was larger than the average in commercial magnetic resonance imaging (MRI) machines. In 15 μm/s fluid velocity, clusters of nanobowls deviate 15° due to magnetic force (FIG. 27C), CA fluorescence (dashed), and bulk temperature (solid) measurements upon AMF stimulation to 0.05 mg/ml magnetic nanobowl (15 μg/mL IO). Three wells were measured for each condition, with and without AMF stimulus. Bulk temperature is similar under both conditions, while significantly higher fluorescence is seen upon AMF stimulus. AMF strength for (FIG. 27A) and (FIG. 27C) is 90 kHz, 18 mT. FIG. 27D is a schematic representation to demonstrate the application of non-invasive localized AMF in animals.

FIG. 28 shows ultrasound (upper panel) and photoacoustic (red) imaging in vitro condition using 3 nm seed and further deposition of different amounts of gold.

FIG. 29 shows time dependent release of siRNA from nanobowls in D PBS buffer with 500 mM DTT reducing agent maintained at 37° C. Representative data (scatter plot) and asymptotic exponential fit (red) are shown. Loading data shows that maximum loading was achieved at 8.17 μg/mg nanobowl. All measurements done in Nanodrop.

FIGS. 30A-30B show in vitro magnetic field-induced uptake and localization of magnetic nanobowls in HEK cells without (control: FIG. 30A, left; FIG. 30B, top panel) and with (experimental: FIG. 30A, right; FIG. 30B, bottom panel) magnetic field after 2 hours of exposure. FIG. 30A shows HEK cells grown in 35 mm cell culture dish at 70% confluence were exposed to fluorescently labeled magnetic nanobowls. In the experimental group, a ½×¼×¼ inch neodymium rare earth ring/donut magnet was placed under the coverslip to pull magnetic nanobowls inside the cells. In the control group, no magnet force was placed. FIG. 30B shows uptake of nanobowls by HEK cells in the presence of a magnetic field. Phase (i), fluorescence in 488 nm channel (ii), and overlay images (iii) depicted were taken with a 10× objective.

FIG. 31A shows MNB injected in mouse brain (top) against PBS as control (bottom). FIG. 31B shows GFAP activity of MNB against PBS. FIG. 31A shows accumulation and clearance of MNB. FIG. 31A shows neural inflammation induced by MNB. FIG. 31E shows MNB accumulation in brain. FIG. 31F shows radiant efficiency in brain, kidney, and liver. FIG. 31A shows distribution of MNB.

FIG. 32A is a diagram showing the design of a nanobowl loaded with siRNA and liposomes containing dexamethasone. FIG. 32B shows an EM image of a nanobowl with a pore and magnetic particles on the outer surface. These magnetic nanobowls are enclosed in a pH sensitive liposome for pH-dependent release of payload.

FIG. 33 shows a diagram illustrating the synthesis of a paramagnetic nanobowl for drug loading and magnetic guided delivery and drug release (top panel), as well as an EM image of nanobowls containing paramagnetic magnetic nanoparticles.

FIG. 34 is a graph showing size changes in nanobowl diameter measured using a DLS instrument comparing nanobowls with paramagnetic magnetic nanoparticles (Fe-J NB) to non-functionalized nanobowls (J N B).

FIG. 35 shows time-dependent release of siRNA adsorbed onto nanobowls through various mechanisms as indicated in the presence of 400 mM DTT reducing agent at 37° C.

FIGS. 36A-36B show in vitro uptake of the siRNA-nanobowl complex. FIG. 36A shows siRNA-nanobowl treated cells; FIG. 36B shows nanobowl treated cells only. HEK cells were treated with 0.5 mg/ml nanobowls loaded with siRNA. Total dosage of siRNA per sample was ˜8 μg/ml. Cells were incubated for 4 hours post-treatment, then washed and fixed in 4% PFA. Control wells got 0.5 mg/ml nanobowls with no siRNA. The siRNA has Cy5 dye which was used for the imaging.

FIG. 37 shows the structure of dexamethasone tagged with FITC.

FIG. 38 shows 48-hour FITC-tagged dexamethasone release from nanobowls in vitro at 37° C. More efficient heat-mediated release of dexamethasone from nanobowls with magnetic ion particle coating (nanobowl-IONP, circles) was observed compared to non-magnetic nanobowls (nanobowl, squares).

FIGS. 39-40 show cell viability in response to treatment with siRNA and dexamethasone in a silica nanobowl (FIG. 39, nanobowl) or magnetic silica nanobowl (FIG. 40, IONP-nanobowl) in HEK cells containing specific cell receptors. Viability data was collected using MTT assay on HEK 298 cell line. X-axis represents nanobowl concentration (mg/ml). A 0 mg/ml concentration data point represents a healthy cell without any nanobowl treatment as control. All data is normalized to the viability of the control cell population. Both nanobowls were pre-loaded with siRNA and dexamethasone.

### DETAILED DESCRIPTION

The SARS-CoV-2 pandemic poses a considerable personal, economic, and societal toll and threatens a worldwide economic collapse. More alarming are the numbers of COVID-19 infection-related fatalities and the long term health impact due to asymptomatic abnormalities. However, there is no effective treatment to date. The WHO supports repurposing of existing drugs such as remdesivir (nucleotide analog for adenosine), lopinavir/ritonavir (protease inhibitor), and anti-inflammatory steroids. These drugs, however, are used at high doses with untoward off-target effects.

As SARS-CoV-2 is a single-stranded RNA virus, it is possible to select several conserved regions of its genome and develop therapies using siRNAs that target the viral genome and intervene viral replication at the genetic level. However, development of effective siRNA therapy is limited by poor targeted delivery in in vivo conditions. Among available viral and non-viral delivery systems, none is able to deliver to all kinds of cell types without limitations and/or side effects.

To be effective, therapeutic agents (e.g., drugs, siRNAs, stem cells, antibodies) must be administered efficiently with no-to-minimal side effects. Current drug delivery systems with constant-rate, zero-order release are inadequate to meet the cyclic or irregular drug requirement in whole organisms. Moreover, systemic and non-targeted delivery systems require a higher dose with potentially multi-organ side effects. Thus, there is a need for new regimes of on-demand therapeutic delivery with tissue (and cell)-specific targeting strategies. Several micro and nano therapeutic delivery systems have been developed using organic and inorganic matrices, with targeted delivery using antibodies or homing molecules. However, these systems have inherent limitations which preclude their effective clinical application.

Treatment of SARS-CoV-2 related infections and diseases is further complicated by the preexisting health conditions of the subjects (e.g., chronic lung, liver, and kidney diseases; asthma; heart disease; diabetes; and compromised immunity). Thus, selective delivery of repurposed drugs and RNA/DNA-based therapeutics to target sites through a focused, on-demand, and image-guided (i.e., trackable) delivery system will ensure the local effective dose and minimize systemic distribution with untoward effects.

While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

To the extent any materials incorporated herein by reference conflict with the present disclosure, the present disclosure controls.

### Definitions

Unless otherwise specified, each of the following terms has the meaning set forth in this section.

The indefinite articles “a” and “an” denote at least one of the associated noun and are used interchangeably with the terms “at least one” and “one or more.”

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (V_{H}) and light (V_{L}) chains of the immunoglobulin with a short linker peptide. Similarly, a disulfide linked Fv antibody can be generated by linking the V_{H} and V_{L} using an interdomain disulfide bond. On the other hand, sdAbs consist of only the variable region from either the heavy or light chain and usually are the smallest antigen-binding fragments of antibodies. A VHH antibody is the antigen-binding fragment of heavy chain only. A diabody is a dimer of scFv fragment that consists of the V_{H} and V_{L} regions noncovalently connected by a small peptide linker or covalently linked to each other. The antibodies disclosed herein, including those that comprise an immunogenically active portion of an immunoglobulin molecule, retain the ability to bind a specific antigen.

The term “antigen” refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

The term “epitope” includes any molecule, structure, amino acid sequence, or protein determinant that is recognized and specifically bound by a cognate binding molecule, such as an antibody or a T cell receptor, or other binding molecule, domain, or protein.

The term “including” is used interchangeably with the term “including, but not limited to.”

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “prevent,” “preventing,” or “prevention” in relation to a given disease, disorder, or viral infection means preventing the onset of disease, disorder, or viral infection development if none had occurred; preventing the disease, disorder, or viral infection from occurring in a subject that may be predisposed to the disease, disorder, or viral infection but has not yet been diagnosed as having the disease, disorder, or viral infection; and/or preventing further disease/disorder/infection development if already present.

The term “subject” refers to a mammalian subject, preferably a human. A “subject in need thereof” refers to a subject who has been infected with an RNA virus, for example, a coronavirus (e.g., SARS-CoV-2), has been diagnosed with a disease caused by an RNA virus, or is at an increased risk of infection or developing a severe illness caused by a coronavirus. The phrases “subject” and “patient” are used interchangeably herein.

The term “treat,” “treating,” or “treatment” in relation to a given disease, disorder, or viral infection (e.g., COVID-19 and/or SARS-CoV-2 infection), includes, but is not limited to, inhibiting the disease, disorder, or viral infection, for example, arresting the development of the disease, disorder, or viral infection; relieving the disease, disorder, or viral infection, for example, causing regression of the disease, disorder, or viral infection; or relieving a condition caused by or resulting from the disease, disorder, or viral infection, for example, relieving or treating symptoms of the disease, disorder, or viral infection.

A “therapeutically effective amount” as used herein is an amount that produces a desired effect in a subject for a disease, disorder, or viral infection (e.g., COVID-19 and/or SARS-CoV-2 infection). In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability); the physiological condition of the subject (e.g., age, body weight, sex, disease type and stage, medical history, general physical condition, responsiveness to a given dosage, and other present medications); the nature of any pharmaceutically acceptable carriers, excipients, and preservatives in the composition; and the route of administration. One skilled in the clinical and pharmacological arts will be able to determine a therapeutically effective amount through routine experimentation, namely, by monitoring a subject's response to administration of the therapeutic composition and adjusting the dosage accordingly. For additional guidance, see, for example, Remington: The Science and Practice of Pharmacy, 22 nd Edition, Pharmaceutical Press, London, 2012, and Goodman & Gilman's The Pharmacological Basis of Therapeutics, 12th Edition, McGraw-Hill, New York, NY, 2011, the entire disclosures of which are incorporated by reference herein.

The terms “SARS-CoV-2,” “COVID,” and “COVID-19” are used interchangeably throughout the present disclosure.

### Nanobowl-Based Therapeutic System and Compositions Thereof

In some aspects, the present technology provides a nanobowl-based therapeutic system. In some embodiments, the nanobowl-based therapeutic system comprises a nanobowl and one or more nucleic acids (e.g., siRNAs) targeting a disease-causing virus. In some embodiments, the virus is a coronavirus. Non-limiting examples of disease-causing coronaviruses include SARS-CoV, MERS-CoV, and SARS-CoV-2, and variants thereof. In some embodiments, the coronavirus is the SARS-CoV-2 coronavirus or its variants, including, for example, the alpha variant (B.1.1.7), and beta variant (B.1.351), the gamma variant (P.1), the delta variant (B.1.617.2), the lambda variant (C.37), the mu variant (B.1.621), the kappa variant (B.1.617.1), the iota variant (B.1.526), the eta variant (B.1.525), the epsilon variant (B.1.427/111.429), the zeta variant (P.2), and the theta variant (P.3).

In some embodiments, the nanobowl-based therapeutic system of the present technology comprises a nanobowl for targeted and controlled delivery of therapeutics (e.g., siRNAs and/or drugs targeting the SARS-CoV-2 virus or a variant thereof). The nanobowl utilized in the present technology may be similar to those described in International Patent Publication No. 2015/192149 titled “Nanostructured carriers for guided and targeted on-demand substance delivery,” the entire disclosures of which are incorporated by reference herein. As the name suggests, the nanobowl may be a hollow “bowl” shaped nanocarrier having a silica core suitable for customization and loading of therapeutic agents. For example, the nanobowl may comprise silica-magnetic capsules, silica-gold magnetic nanogold bowls, or silica-gold magnetic nanobowls. These magnetically guided, stimuli-responsive, polymer-gated, multifunctional, theragnostic delivery-enabled nanobowls allow controlled on-off cargo release using external and internal stimuli, such as magnetic field, heat, pH, and biochemical manipulations. The nanobowl delivery system has a flexible modular design allowing rapid adaptation and integration for specific diagnostic and/or therapeutic applications, making this an ideal platform for development of therapeutic applications. The outer surface can be tailored or functionalized for target (e.g., cell, tissue) recognition or for capturing and encapsulating external biomolecules. The inner cavity can be tailored for defined payload capacity which would be unfeasible for currently available nanoparticle-based delivery systems. The gold and iron particles allow on-off release of the payload by RF magnetic heating or near infrared heating (NIR), as well as photoacoustic, ultrasound, or MRI to track the delivery system.

In some embodiments, the nanobowl of the nanobowl-based therapeutic system may be made of organic or inorganic materials suitable for therapeutic applications. In some embodiments, the nanobowl is made of inorganic material, for example, silica (also known as silicon dioxide) or derivatives thereof (e.g., tetraethyl orthosilicate (TEOS)). The nanobowl may be porous or non-porous. As the name suggests, the nanobowl may be a hollow “bowl” shaped with an interior surface and an exterior surface. The interior surface and/or exterior surface may be functionalized in the same way or differently to accommodate loading of therapeutic agents.

In some embodiments, the nanobowl of the present technology may be further functionalized and/or comprise one or more surface modifications to improve its functionality. In some embodiments, the nanobowl may be made magnetic or thermally sensible by attaching gold and/or iron oxide (IO) nanoparticles on the surface of the nanobowl. Or, in other embodiments, the gold and/or IO nanoparticles may be dispersed in the silica core of the nanobowl. The gold and/or IO coating can facilitate thermally activable release of drug payload upon application of a magnetic field.

In some embodiments, the nanobowl may be coated with a heat-sensitive coating to allow thermally controllable release of drug payload. A non-limiting example of such heat-sensitive coating includes N-isopropylacrylamide (NIPAM). A heat-sensitive coating, such as an NIPAM coating, can protect the payload from interacting with the environment, prevent spontaneous leaking, as well as enable conditional delivery in response to a specific temperature. After delivery to the target site, application of an environmental change (e.g., a shift in temperature, magnetic fields) can induce magnetic hyperthermia and change the permeability properties of the coating to achieve controlled release of the payload.

In some embodiments, the nanobowl may be coated with a biodegradable coating. Non-limiting examples of biodegradable polymers include polylactic-polyglycolic acid (PLGA) and p(MMAco-NIPAM). Biodegradable coating offers the advantage that when the payload is released, the coating can bio-degrade to allow disposition of the nanobowl by the body.

In some embodiments, the nanobowl may be coated with a lipid coating or encapsulated by a liposome to allow protection against immune responses, spontaneous leakage, and blood shear force. The coating or encapsulation may also facilitate endocytosis, that is, the incorporation of the nanobowl into a cell. In some embodiments, the lipid or liposome may comprise 1,2-dioleoyl-sn-glycero-3-phosphoethanolamine (18:1 (Δ9-Cis) PE or DOPE), 1,2-dioleoyl-3-trimethylammonium-propane (18:1TAP or DOTAP). These lipids can increase stability of the nanobowl, facilitate uptake of encapsulated nanobowls by cells and/or release of drug loads due to their ability to fuse with cellular lipid bilayers and vesicular compartments. In some embodiments, the liposome may comprise 1,2-Dipalmitoyl-sn-glycero-3-phosphocholine.

In some embodiments, the nanobowl-based therapeutic system of the present technology comprises one or more nucleic acids targeting the genome of a virus, including a coronavirus, such as the SARS-CoV-2 virus or a variant thereof. The one or more nucleic acids may target the same virus or different viruses, respectively. In some embodiments, the nucleic acids may be DNA or RNA molecules. In some embodiments, the nucleic acids may be circular or linear.

In some embodiments, the nucleic acids may be short interfering RNAs (siRNAs). SiRNAs, also known as silencing RNAs, are a class of double-stranded RNAs (dsRNAs) typically 19-27 base pairs in length and operating within the RNA interference (RNAi) pathway for gene silencing based on sequence complementarity. In some embodiments, the siRNAs of the present technology can be a dsRNA comprising a hairpin structure, or alternatively, a dsRNA without the hairpin structure. In some embodiments, the siRNAs of the present technology may be 19-27 base pairs in length, for example, 19-20 base pairs in length.

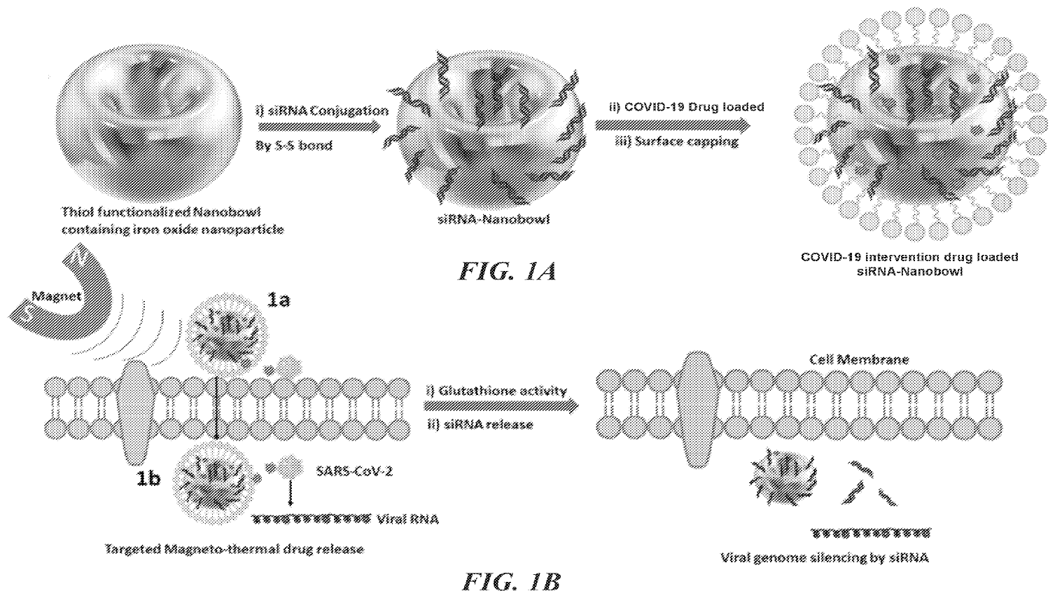

In some embodiments, the siRNA may comprise or consist of a nucleotide sequence that is identical or complementary to a genetic sequence of a virus, including a coronavirus, such as the SARS-CoV-2 virus or a variant thereof. The genetic sequence can be a conserved sequence among different strains of the virus. For example, the siRNAs may target conserved regions of different SARS-CoV-2 viral strains (FIG. 1). In some embodiments, the siRNAs may target the Orf1ab, S, M, and/or N gene regions of the SARS-CoV-2 genome.

In some embodiments, the siRNA may comprise or consist of a nucleotide sequence set forth in any one of SEQ ID NOs: 1-7 or at least 80% identical (e.g., at least 80%, at least 85%, at least 90%, at least 95%, at least 96%, at least 97%, at least 98%, at least 99%, or 100% identical) to any one of SEQ ID NOs: 1-7. In some embodiments, the siRNA may comprise or consist of a nucleotide sequence complimentary to the nucleotide sequence set forth in any one of SEQ ID NOs: 1-7 or at least 80% identical (e.g., at least 80%, at least 85%, at least 90%, at least 95%, at least 96%, at least 97%, at least 98%, at least 99%, or 100% identical) to any one of SEQ ID NOs: 1-7.

In some embodiments, the nucleic acids (e.g., siRNAs) of the present technology can be attached or conjugated with the nanobowl, for example, through covalent bonds. In some embodiments, the surface of the nanobowl may be functionalized, for example, by chemical modifications using reagents known to a person of skill in the art, to enable covalent linkage to the nucleic acids. Non-limiting examples of such chemical modifications include functionalization with amine groups, carboxyl groups, and azide groups. In some embodiments, the siRNAs are conjugated to the nanobowl through disulfide bonds. Certain cellular enzymes are able to break down disulfide bonds, thereby facilitating siRNA release from the nanobowl carrier inside a cell.

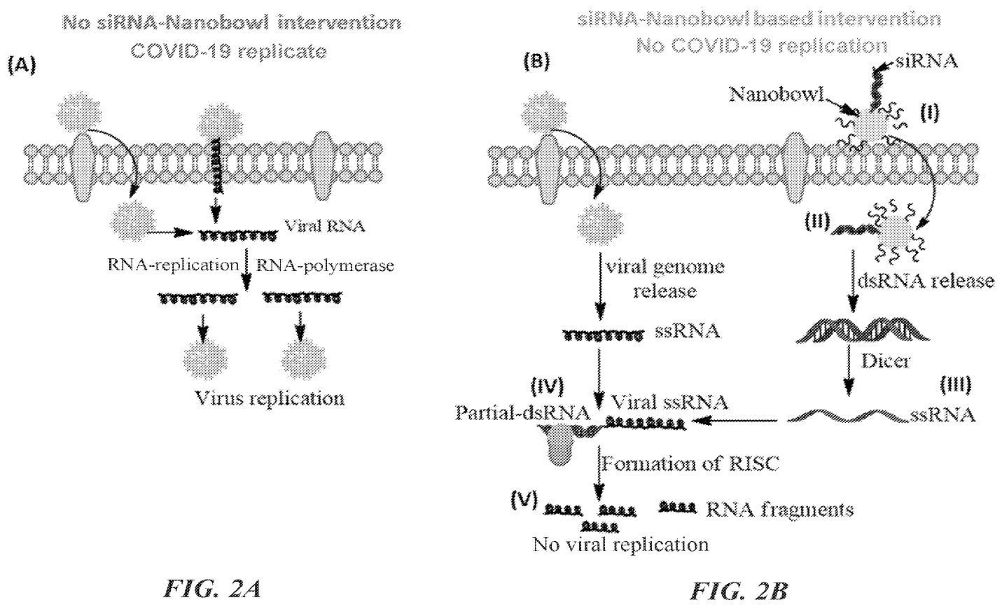

In some embodiments, the nanobowl-based therapeutic system of the present technology involves the following aspects or mechanisms of function: (1) identification of 19-nucleotide dsRNA or siRNA, with or without hairpin, containing several conserved regions of SARS-CoV-2 strains; (2) conjugation of siRNA with a nanobowl, a highly efficient delivery/transfection vehicle, using disulfide bonds (S—S); (3) targeted delivery of siRNA-nanobowl and localization to target site using external magnetic field; (4) release of siRNA via S—S bond breakage by cellular glutathione; (5) processing of siRNA by cellular machinery into single-stranded siRNA and hybridization of the singled-stranded siRNA with conserved regions of COVID-19 viral RNA; and (6) fragmentation of viral genome by cellular molecular machinery (e.g., the RNA-induced silencing complex (RISC)) and inhibition of COVID-19 replication or multiplication (FIG. 2).

In some embodiments, the nanobowl-based therapeutic system of the present technology further comprises one or more additional therapeutic agents for the treatment of SARS-CoV-2 infection or COVID-19. The one or more additional therapeutic agents can be loaded to the nanobowl (e.g., loaded into the hollow cavity, to the internal surface, or to the exterior surface of the nanobowl) using approaches suitable for the physicochemical natures of the payload and the nanobowl surface for controlled and targeted delivery and release in a subject, in combination with the siRNA therapy.

In some embodiments, the one or more additional therapeutic agents comprise an antiviral agent, an anti-inflammatory agent, an antimalaria agent, and/or a biologic agent. In some embodiments, the antiviral agent is remdesivir (e.g., Veklury®); favipiravir (e.g., Avigan®); lopinavir/ritonavir (e.g., Kaletra®, Aluvia®); nitazoxanide (e.g., Alinia®); danoprevir (e.g., Ganovo®); umifenovir (e.g., Arbidol®); nafamostat, brequinar, merimepodib, molnupiravir, opaganib (e.g., Yeliva®); and/or ivermectin (e.g., Soolantra®, Stromectol®, Sklice®). In some embodiments, the anti-inflammatory agent is ruxolitinib (e.g., Jakafi®); baricitinib (e.g., Olumiant®); dapagliflozin (e.g., Farxiga®); eicosapentaenoic acid (EPA, in free acid or ethyl ester form, e.g., Lovaza®, Epadel®, Vascepa®); tocilizumab (e.g., Actemra®); sarilumab (e.g., Kevzara®); ravulizumab (e.g., Ultomiris®; losmapimod, pacritinib, bucillamine, tradipitant, lenzilumab, acalabrutinib (e.g., Calquence®); otilimab, abivertinib maleate, selinexor (e.g., Xpovio®); brequinar, ibudilast, apilimod dimesylate, gimsilumab, dociparastat sodium, itolizumab (Alzumab™); pemziviptadil, prednisolone, dexamethasone, reparixin, brensocatib, emapalumab, and/or anakinra. In some embodiments, the antimalaria agent is hydroxychloroquine or chloroquine. In some embodiments, the biologic agent is an antibody, for example, an antibody recognizing the SARS-CoV-2 coronavirus. In these embodiments, the antibody may recognize at least a portion of the SARS-CoV-2 virus, such as an epitope on a spike protein. In some embodiments, the biological agent is a vaccine, for example, a vaccine for the SARS-CoV-2 coronavirus.

In some embodiments, the present technology based on the combination of nanobowls and siRNA molecules for hybridization with viral RNA can achieve the following features: (1) the performance of siRNA specific for SARS-CoV-2 conserved regions; (2) the efficient delivery of siRNA to the target site to induce viral genetic target binding and to activate the cellular machinery and fragmentation of the viral RNA, thereby inhibiting viral multiplication; (3) siRNA conjugation with the nanobowl to enable efficient delivery of the siRNA to the target and to mitigate inherent limitations of siRNA delivery; (4) the functionalized nanobowl system capable of controlled delivery and release of one or more COVID-19 drugs, such as monoclonal antibodies, mesylate, lopinavir/ritonavir, rem desivir, for combination COVID-19 treatment with siRNA intervention.

As shown in more details in the working examples, research using different drugs, cells lines, and animal models show that the nanobowl delivery system can deliver drugs, DNA/RNA, and small molecules using pH and/or heat-mediated on-off release, and it is non-toxic in animal models. This delivery platform may also include the following features: (1) hollow-sphere nanocapsules containing defined insertion of theragnostic biological agents; (2) magnetic nanoparticles embedded between silica and gold concentric two shells; (3) transport of these carriers by magnetic vector force and by specific targeting ligands in pre-defined tissues (e.g., brain, lung, heart); (4) controlled release of the genetic cargo, drugs, imaging contrast agent in on-off switch mode by a remotely applied RF field, NIR, or pH-change; and (5) the continuous monitoring of their traverse within the body (e.g., by using photoacoustic imaging). This innovative platform technology for a wireless-controlled, magnetically guided, on-demand theragnostic delivery system will provide effective delivery vehicles for therapeutics, as well as contrast/staining reagents to monitor efficacy of administered drug and disease progression, thus contributing to rapid and effective personalized global health. Applied to the treatment of COVID-19, the siRNA-nanobowl (siRNB)-based blockage of viral replication using conserved regions of SARS-CoV-2 strains, as well as the possibility of simultaneous delivery of one or more therapeutics for COVID-19 (e.g., peptides, proteins, antibodies, drugs), could provide an effective treatment system with limited side effects in present and future pandemics.

In some aspects, the nanobowl-based therapeutic system according to various embodiments disclosed herein is present in a composition.

In some embodiments, the composition may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells as disclosed herein further comprise a suitable infusion media.

### Methods of Treatment

In some aspects, the nanobowl-based therapeutic system may be used in the treatment and/or prevention of infections and/or diseases caused by RNA viruses (e.g., coronaviruses) or amelioration of one or more symptoms associated thereof in a subject. Non-limiting examples of infections and/or diseases caused by coronaviruses include SARS (caused by the SARS-CoV virus), MERS (caused by the MERS-CoV virus), and COVID-19 (caused by the SARS-CoV-2 virus and variants thereof). In some embodiments, the infections and/or diseases are caused by the SARS-CoV-2 virus or its variants, including, for example, the alpha variant (B.1.1.7), and beta variant (B.1.351), the gamma variant (P.1), the delta variant (B.1.617.2), the lambda variant (C.37), the mu variant (B.1.621), the kappa variant (B.1.617.1), the iota variant (B.1.526), the eta variant (B.1.525), the epsilon variant (B.1.427/111.429), the zeta variant (P.2), and the theta variant (P.3). In some embodiments, the treatment and/or prevention of infections and/or diseases comprise prevention or inhibition of viral replication or multiplication.

In some embodiments, the methods comprise administering to a subject in need thereof a therapeutically effective amount of the nanobowl-based therapeutic system, or a composition comprising the same, according to various embodiments of the present technology.

In some embodiments, the methods comprise delivering the nanobowl-based therapeutic system, or a composition comprising the same, according to various embodiments of the present technology to a target (e.g., cells, tissues, organs) inside of the subject through the application of external stimuli. A non-limiting example of such external stimuli includes a magnetic field.

In some embodiments, the methods comprise releasing the siRNA and/or one or more therapeutic agents from the nanobowl-based therapeutic system at the target site (e.g., inside a target cell) in a controlled manner through the application of internal or external stimuli. Non-limiting examples of such internal stimuli include a biochemical substance (e.g., a biochemical substance present inside the target cell). Non-limiting examples of such external stimuli include magnetic field, light, heat, and pH.

In some embodiments, the nanobowl-based therapeutic system, or a composition comprising the same, is administered to the subject in a range of from about 1 mg/kg to about 500 mg/kg, from 10 mg/kg to about 150 mg/kg, from 30 mg/kg to about 120 mg/kg, from 60 mg/kg to about 90 mg/kg, for example, at a dose of about 15 mg/kg, about mg/kg, about 45 mg/kg, about 60 mg/kg, about 75 mg/kg, about 90 mg/kg, about 105 mg/kg, about 120 mg/kg, about 135 mg/kg, about 150 mg/kg, or more. In some embodiments, the nanobowl-based therapeutic system, or a composition comprising the same, is administered to the subject to provide a daily dose of up to about 0.5 g, about 1 g, about 2 g, about 3 g, about 4 g, about 5 g, about 6 g, about 7 g, about 8 g, about 9 g, about g, about 11 g, about 12 g, about 13 g, about 14 g, about 15 g, about 16 g, about 17 g, about 18 g, about 19 g, about 20 g, or more. For example, the nanobowl-based therapeutic system, or a composition comprising the same, may be administered in an amount sufficient to provide a daily dose of about 50 mg to about 10000 mg, about 100 mg to about 7500 mg, or about 100 mg to about 5000 mg; for example, about 50 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, about 4000 mg, about 4100 mg, about 4200 mg, about 4300 mg, about 4400 mg, about 4500 mg, about 4600 mg, about 4700 mg, about 4800 mg, about 4900 mg, about 5000 mg, about 5100 mg, about 5200 mg, about 5300 mg, about 5400 mg, about 5500 mg, about 5600 mg, about 5700 mg, about 5800 mg, about 5900 mg, about 6000 mg, about 6100 mg, about 6200 mg, about 6300 mg, about 6400 mg, about 6500 mg, about 6600 mg, about 6700 mg, about 6800 mg, about 6900 mg, about 7000 mg, about 7100 mg, about 7200 mg, about 7300 mg, about 7400 mg, about 7500 mg, about 7600 mg, about 7700 mg, about 7800 mg, about 7900 mg, about 8000 mg, about 8100 mg, about 8200 mg, about 8300 mg, about 8400 mg, about 8500 mg, about 8600 mg, about 8700 mg, about 8800 mg, about 8900 mg, about 9000 mg, about 9100 mg, about 9200 mg, about 9300 mg, about 9400 mg, about 9500 mg, about 9600 mg, about 9700 mg, about 9800 mg, about 9900 mg, or about 10000 mg.

In some embodiments, the nanobowl-based therapeutic system, or a composition comprising the same, may be administered in a manner appropriate to the disease, condition, or disorder to be treated as determined by persons skilled in the medical art, for example, inhalation, oral administration, subcutaneous administration, intravenous administration, intramuscular administration, intradermal administration, intrathecal administration, intratracheal administration, or intraperitoneal administration.

In some embodiments, the nanobowl-based therapeutic system, or a composition comprising the same, may be administered to the subject once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the nanobowl-based therapeutic system, or a composition comprising the same, may be administered every day, every other day, every third day, weekly, biweekly (i.e., every other week), every third week, monthly, every other month, or every third month.

In some embodiments, the nanobowl-based therapeutic system, or a composition comprising the same, may be administered over a predetermined time period. Alternatively, the nanobowl-based therapeutic system, or a composition comprising the same, may be administered until a particular therapeutic benchmark is reached. In some embodiments, the methods provided herein include a step of evaluating one or more therapeutic benchmarks in a biological sample, such as, but not limited to, the presence or absence of a virus or symptoms associated thereof, to determine whether to continue administration of the nanobowl-based therapeutic system or a composition comprising the same.

In some embodiments, the methods further comprise administering to the subject a pharmaceutically effective amount of one or more additional therapeutic agents as described to obtain improved or synergistic therapeutic effects. In some embodiments, the subject is administered the one or more additional therapeutic agents before administration of the nanobowl-based therapeutic system or a composition comprising the same. In some embodiments, the subject is co-administered the one or more additional therapeutic agents and the nanobowl-based therapeutic system, or a composition comprising the same. In some embodiments, the subject is administered the one or more additional therapeutic agents after administration of the nanobowl-based therapeutic system, or a composition comprising the same.

In some embodiments, the one or more additional therapeutic agents comprise an antiviral agent, an anti-inflammatory agent, an antimalaria agent, and/or a biologic agent. In some embodiments, the antiviral agent is remdesivir (e.g., Veklury®); favipiravir (e.g., Avigan®); lopinavir/ritonavir (e.g., Kaletra®, Aluvia®); nitazoxanide (e.g., Alinia®); danoprevir (e.g., Ganovo®); umifenovir (e.g., Arbidol®); nafamostat, brequinar, merimepodib, molnupiravir, opaganib (e.g., Yeliva®); and/or ivermectin (e.g., Soolantra®, Stromectol®, Sklice®). In some embodiments, the anti-inflammatory agent is ruxolitinib (e.g., Jakafi®); baricitinib (e.g., Olumiant®); dapagliflozin (e.g., Farxiga®); EPA (in free acid or ethyl ester form, e.g., Lovaza®, Epadel®, Vascepa®); tocilizumab (e.g., Actemra®); sarilumab (e.g., Kevzara®); ravulizumab (e.g., Ultomiris®); losmapimod, pacritinib, bucillamine, tradipitant, lenzilumab, acalabrutinib (e.g., Calquence®); otilimab, abivertinib maleate, selinexor (e.g., Xpovio®); brequinar, ibudilast, apilimod dimesylate, gimsilumab, dociparastat sodium, itolizumab (Alzumab™); pemziviptadil, prednisolone, dexamethasone, reparixin, brensocatib, emapalumab, and/or anakinra. In some embodiments, the antimalaria agent is hydroxychloroquine or chloroquine. In some embodiments, the biologic agent is an antibody, for example, an antibody recognizing the SARS-CoV-2 coronavirus. In some embodiments, the biological agent is a vaccine, for example, a vaccine for the SARS-CoV-2 coronavirus.

As one of ordinary skill in the art would understand, the one or more additional therapeutic agents and the nanobowl-based therapeutic system or a composition comprising the same can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

### Example 1. Lipid-Encapsulated Silica Nanobowls as an Efficient and Versatile DNA Delivery System

Non-mesoporous Janus silica nanobowls (nanobowls) are unique in that they possess two different non-porous surfaces per particle for loading biological molecules and can thus be designed with multifunctional properties. Although silica nanobowls have been successfully employed for both targeted therapeutic and diagnostic applications, their ability to deliver DNA has not yet been fully explored. The purpose of this study was to design and develop an in vitro transfection agent that would exploit the distinct characteristics of the silica nanobowl. First, we determined that the nanobowl surface can be linked to either supercoiled cDNA plasm ids or vector-less, linear cDNA constructs. Additionally, the linearized cDNA can be functionalized and chemisorbed on nanobowls in order to obtain a controlled release. Second, the successful transfection of cells studied was dependent on the lipid coating of the nanobowl. Although both nanobowls and LNBs were capable of undergoing endocytosis, nanobowls appeared to remain within vesicles as shown by TEM. Third, fluorescence microscopy and Western blotting assays revealed that transfection of four different cell lines and acutely isolated rat sensory neurons with LNBs loaded with either linear or supercoiled cDNA constructs coding for the fluorescent protein, clover and tdT, resulted in protein expression. Fourth, two separate opioid receptor-ion channel signaling pathways were functionally reconstituted in HEK cells transfected with LNBs loaded with three separate cDNA constructs. Overall, these results lay the foundation for the use and further development of LNBs as in vitro transfection agents.

### Introduction

Nanomaterials have large surface area to volume ratios, and their porosity allows for high DNA condensation efficiencies. In addition to low cost and scalable synthesis, nano vectors possess other favorable attributes. For instance, their size, shape, surface chemistry, optical, and magnetic properties are tunable. Furthermore, their biocompatibility and stealth properties allow for a reduced immune recognition and efficient cellular internalization. Organic nanomaterials, employed for in vitro gene delivery, include solid lipid, polymeric, hydrogel nanoparticles and dendrimers. Inorganic nanoparticles are attractive candidates for DNA delivery as they have robust structures that can retain their shape and chemical properties upon extended exposure to the biological milieu. Moreover, inorganic nanoparticles possess optical and magnetic properties that can be exploited for simultaneous tracking and diagnostic applications.

Among inorganic nanomaterials, silica nanomaterials are particularly useful for DNA delivery due to their chemical inertness, low cytotoxicity, low cost, controllable porosities and shapes, and surface chemistry that is easily engineered. Additionally, silica nanomaterials are capable of maintaining their physical robustness in both solution and dried forms for long term storage. Both microporous and mesoporous silica nanostructures have been previously employed in in vitro gene delivery. Silica nanobowls are a new class of Janus nanoparticles with an engineered cavity to hold different types of payloads. The outside and inside surfaces of the cavity can be differentially functionalized to add stabilizing polymers like polyethylene glycol (PEG), specific targeting moieties and special properties like ferromagnetism and plasmonic scattering. However, the use of non-mesoporous silica nanobowls as gene delivery vehicles has not yet been explored.

Despite their high transfection efficiency in dividing cells under in vitro conditions, inorganic nanomaterials have had limited success in transfection of non-dividing cells, such as neurons. Neuron transfection approaches typically employ viruses, physical non-viral techniques (i.e., nuclear or cytoplasmic injections, electroporation and magnetofection), and chemical techniques (i.e., lipofection or PEI). The physical techniques have high efficiencies, though not scalable to in vivo applications, while chemical techniques can be toxic for non-dividing cells. This presents a unique opportunity to design nanomaterials as transfection agents for both dividing and non-dividing cells with a high transfection efficiency and minimal cytotoxicity compared to current transfection technologies. Employment of nanomaterials, including the inorganic type, has had limited use for transfection of neurons. Nevertheless, there are reports of in vivo gene delivery to the brain.

In this study, we describe the development of DNA-loaded silica nanobowls (nanobowls) that can be internalized within 4 hours in immortalized mammalian cell lines and in acutely dissociated rat peripheral neurons. We also show that the successful transfection (i.e., release of DNA) relied on coating the nanobowls with “helper” lipids. These nanovectors can be engineered to physisorb or chemisorb DNA at high loading efficiencies. We further demonstrate that the nanobowls are capable of transfecting cells when loaded with either linearized or supercoiled cDNA constructs. Finally, we show that these lipid-coated silica nanobowls can simultaneously deliver three cDNA constructs to recapitulate the coupling mechanism of G protein-coupled receptors (i.e., opioid receptors) and ion channels in an in vitro model.

### Experimental Procedures

Nanobowl Synthesis and Functionalization

Nanobowls were synthesized with 100 nm carboxyl terminated PS spheres (Polysciences, Inc.) as templates, as either large scale (60 ml) or small scale (6 ml). Briefly, 7 ml (or 0.7 m 1) deionized water, 40 m 1 (or 4 m 1) isopropyl alcohol (Sigma-Aldrich), and 13 ml (1.3 ml) ammonium hydroxide (Sigma-Aldrich) were magnetically stirred together. Thereafter, 550 μl (55 μl) tetraethyl orthosilicate (TEOS, Sigma-Aldrich ≥99% purity) and 1 ml (100 μl) PS spheres (2.5% solids w/v) were added simultaneously to the above mixture and allowed to react by stirring at high speed for 2 hours at room temperature. The solution was then centrifuged at 500 g for 10 minutes to separate large aggregates formed during nanobowl synthesis. The supernatant containing single dispersed nanobowls were washed 3 times in ethanol (EtOH, Sigma-Aldrich) by centrifugation at 3221 g for 15 minutes in order to precipitate single nanobowls. The purified nanobowls were re-dispersed in EtOH and allowed to air dry overnight. Our laboratory previously reported these features in which we found that 70-95% of the synthesized product are Janus nanobowls, with approximately 30% single cavity, approximately 40% double cavity and about 30% nanobowls with >2 cavities (i.e., >2 PS cores incorporated). After synthesis, the dried nanobowls were re-dispersed (1 mg/ml) in anhydrous dim ethyl formamide (DMF, Sigma-Aldrich) and heated for 3 hours in a silicone oil bath at 60° C. with magnetic stirring in order to dissolve the PS template and expose the cavity. The nanobowls were next washed 4 times in EtOH and air-dried. To amine functionalize for cDNA loading, the dried DMF-washed nanobowls were re-dispersed in a 1:1 ethanol:toluene (Sigma-Aldrich) bath at 0.5 mg/ml and allowed to react with 10 mM 3-aminopropyltriethoxysilane (APTES, Sigma-Aldrich) at 60° C. for 3 hours under rapid magnetic stirring. The nanobowls were then washed 4 times in EtOH and air-dried in preparation for DNA loading. In another set of experiments, Stöber silica nanoparticles were synthesized as previously described without the use of a PS template and APTES-modified as described above for nanobowl to allow for cDNA loading.

Thermogravimetric Analysis (TGA)

Purified amine-coated and DMF-washed nanobowls were air-dried until a white powder was formed. Approximately 4-5 mg of material were placed into a high-temperature platinum sample pan and incubated in a furnace. A 5 minute purge of inert gas at room temperature preceded the measurements to remove air from the furnace. Balance flow and furnace flow were both set to 25 ml/minute which resulted in 100 ml/minute of total gas flow through the sample. The temperature was equilibrated at 100° C. and the sample was heated to 1000° C. at 10° C./minute. All measurements were performed in a Discovery TGA-MS (TA Instruments). The weight loss (%) was calculated with respect to starting weight at room temperature before furnace temperature was ramped up. All measurements were performed in Materials Characterization Lab, Materials Research Institute, Penn State University, PA. The results were analyzed similarly to those previously described.

DNA Linearization and Functionalization

For cDNA loading and transfections, we chose clover (vector: pcDNA 3.1) and tdT (vector: pEGFP-N1), which code for two high quantum efficiency fluorescent proteins of the GFP family. PCR was performed to introduce either amine or azide functional groups into linear DNA. Forward primers with appropriate functional groups were designed to hybridize at the start of the CMV promoter region of the pcDNA3.1 plasm id containing the clover DNA insert. The modifications at the 5′ end of the forward primer were either a carboxyl or an azide group followed by a disulfide bond. The reverse primer was not modified and was designed to hybridize at the end of the polyadenylation sequence of the plasmid. All primers (Integrated DNA Technologies (IDT)) were custom designed. The sequences of the primers were the following:

Forward primer (FWD):
(SEQ ID NO: 8)
5′-GTTGACATTGATTATTGACTAGTTATTAATAGTAAT-3′.
Reverse primer (REV):
(SEQ ID NO: 9)
5′-CCATAGAGCCCACCGCAT-3′.

### Functionalized Forward Primers:

FWD-Azide:
(SEQ ID NO: 8)
5′ N₃-Cₙ-S-S-Cₙ-GTTGACATTGATTATTGACTAGTTATTAATAGTA
AT-3′.
(IDT modification code: /5AzideN//iThioMC6-D/)
FWD-Carboxyl:
(SEQ ID NO: 8)
5′ HOOC-Cₙ-S-S-Cₙ-GTTGACATTGATTATTGACTAGTTATTAATAG
TAAT-3′.
(IDT modification code: /5Carboxyl//iThioMC6-D/)
tdT primers:
Forward primer (FWD):
(SEQ ID NO: 10)
5′-TAGTTATTAATAGTAATCAATTACGGGGTC-3′.
Reverse primer (REV):
(SEQ ID NO: 11)
5′-GCAGTGAAAAAAATGCTTTATTTGTG-3′.

PCR was performed using the OneTaq HotStart 2×master mix (New England Biolabs). The PCR products were purified using commercially available standard DNA clean and concentrator kits (Zymo Research, 25 μg columns or Qiagen, 10 μg columns) and reconstituted in DNAse, RNase free molecular biology grade water. The purified products were quantified using the Qubit dsDNA BR assay kit (Thermo-Fisher Scientific) as per manufacturer's protocol. Lin-A and Lin-C refer to linearized cDNA products purified from PCR on supercoiled clover cDNA template with REV and FWD-Azide and FWD-Carboxyl, respectively. All linearized PCR products were visualized on agarose gel electrophoresis to confirm product size (clover ˜1.7 kbp & tdT ˜2.4 kbp, data not shown).

Nanobowl-DNA Loading Assay

The dried, amine-coated nanobowls were resuspended in Dulbecco's Phosphate Buffered Saline DPBS (with Ca²⁺ and Mg²⁺; Thermo-Fisher Scientific) with light sonication for 5 minutes at a final 1 mg/ml concentration. In addition, 2-50 μg of supercoiled or linearized cDNA was added to 1 ml (1 mg/ml) of nanobowl-DPBS solution and allowed to bind overnight at 4° C. with gentle shaking. The linkers, N-hydroxysuccinimide (NHS) and 1-ethyl-3-(3-dim ethylaminopropyl)-carbodiimide (EDC; both from Thermo-Fisher Scientific), were employed for chemisorption of Lin-C on nanobowls in 2-(N-Morpholino) ethanesulfonic acid (MES)-buffered saline (Thermo-Fisher Scientific). Initially, Lin-C (10 μg) was pretreated with 2 mM EDC and 5 mM NHS in 0.1 M MES buffer for 30 minutes at room temperature before addition to the 1 mg/ml nanobowl-DPBS solution and overnight incubation. For azido-DBCO click chemistry, the amine-functionalized nanobowls were first conjugated overnight with click chemistry linker dibenzo cyclooctyne, i.e., DBCO-NHS (Click Chemistry Tools) in dimethyl sulfoxide (DMSO, Sigma-Aldrich), washed 4 times in ethanol and dried before DPBS reconstitution at 1 mg/m 1. Afterwards, Lin-A (10 μg) was added to DBCO-coated nanobowls resuspended in DPBS and allowed to mix overnight. Once the DNA was loaded, the nanobowls were centrifuged at 3221 g for 30 minutes and the supernatants were collected for DNA quantification with Qubit assay kit (Thermo-Fisher Scientific). All loading efficiencies (%) were calculated as μg cDNA bound*100/μg cDNA added per mg J NB.

Nanobowl-DNA Release Assay

Amine- and DBCO-functionalized nanobowls were loaded with Lin-C and Lin-A at 10 μg/mg nanobowl as described above. Following an incubation period (24 hours), nanobowls were centrifuged at 3200 g for 30 minutes and the supernatant was then decanted. The linearized cDNA-loaded nanobowls were then gently reconstituted in 500 mM p-mercaptoethanol (Sigma-Aldrich) containing DPBS (with Ca²⁺ and Mg²⁺) at 2 mg/m 1 concentration in a 500 μl final volume. Control nanobowls were loaded with Lin-C or Lin-A reconstituted in DPBS at a final concentration of 2 mg/m 1. Both control and β-mercaptoethanol samples were placed in a heat block (37° C.) for 4, 24, and 48 hours. After each incubation period, the nanobowls were centrifuged at 3200 g for 30 minutes and the supernatant was decanted and used to measure cDNA concentration in triplicate with the Qubit dsDNA assay kit. DPBS with 500 mM β-mercaptoethanol only was confirmed to have no background in the Qubit dsDNA assay.

Nanobowl-DNA Lipid Encapsulation

The lipids 1,2-dioleoyl-sn-glycero-3-phosphoethanolamine (18:1 (Δ9-Cis) PE or DOPE) and 1,2-dioleoyl-3-trimethylammonium-propane (chloride salt) (18:1 TAP or DOTAP; both from Avanti Polar Lipids) were mixed at a 1:1 molar ratio in chloroform and transferred to a pre-etched round bottom glass flask. The chloroform was dried with a gentle nitrogen stream. Thereafter, approximately 1.5 gr of 2 mm glass beads (Sigma-Aldrich) were added to the bottom of the flask and a lipid reconstitution buffer (KCl 100 mM Tris 10 mM HEPES mM pH 8.4) was added to obtain a 1 mg/ml lipid solution. The liposomes were prepared by swirling the flask with the glass beads continuously for 5 minutes. The liposome solution was then serially filtered through 0.45 μm (Pall diagnostics) and 0.22 μm (Pall diagnostics) sterile filters. Afterwards, the liposome solution was extruded through a 0.10 μm filter (Avanti Polar Lipids) using a mini extruder setup with gas tight syringes (Avanti Polar Lipids), with 4 passes through the filter per 1 ml extruded. Finally, 1 ml of 100 nm extruded liposome solution was mixed via gentle shaking with 1 ml nanobowl-DNA conjugate (1 mg/ml) in DPBS for 60 minutes with gentle shaking at room temperature. The nanobowls were then centrifuged and washed once in 1 ml DPBS at 3221 g for 30 minutes. The LNBs were finally resuspended in 1:1 DPBS:Opti-MEM (Thermo-Fisher Scientific) at the desired final LNB concentration for transfection.

Nanobowl Toxicity Assays

MTT Assays

HEK, ND7/23, L-cells, and HeLa cells were purchased from ATCC. The cells were plated at a density of 25,000 cells/well in glass-bottom 96-well plates 24 hours before the experiment. On the day of the experiment, the cells were incubated in 0.05, 0.125, 0.25, and 1.0 mg/ml LNBs in 1:1 DPBS:Opti-MEM (200 μl final volume/well) at 37° C. for 4 hours. The null LNB (Control) group was incubated in vehicle (DPBS:Opti-MEM). Following the incubation period, the wells were gently rinsed in warm DMEM twice and 100 μl of warm DMEM (without phenol red) mixed with 10 μl of 12 mM MTT solution (Vybrant MTT assay kit, Thermo-Fisher Scientific) were added to the wells for 4 hours at 37° C. Thereafter, 85 μl of supernatant per well was discarded and gently replaced with 100 μl DMSO. The plate was incubated for 30 minutes at 37° C. and kept on a rotary shaker for 30 minutes at room temperature to allow the uniform dissolution of form azan. The plates were scanned for absorbance at 540 nm in FlexStation3 microplate reader (Molecular Devices). The absorbances were normalized to the live cell control and converted to percent viability.

Flow Cytometry

HEK cells were plated on 6-well plates at 120,000 cells/well 24 hours prior to the start of the experiment. Cells were incubated with LNB (0.05-1.0 mg/ml) loaded with 10 μg/mg linearized clover for 4 hours in a humidified atmosphere at 37° C. in 5% CO₂/95% air. Each condition was performed in duplicate. Following the incubation period, the wells were rinsed with warm DMEM (without phenol red) and returned to the incubator for an additional 44 hours. The negative control group was incubated in 1 mg/ml LNB in DPBS:Opti-MEM. The positive control group (i.e., clover-expressing cells) was transfected with supercoiled clover cDNA (4 μg) employing Lipofectamine 2000 (Thermo-Fisher Scientific) per well, followed by washing with warm, clear DMEM and 24 hours incubation before analysis. Prior to performing flow cytometry, phase contrast and fluorescence images were obtained with a Nikon TE2000 microscope, an Orca-ER CCD camera (Hamamatsu Photonics), iVision software for acquisition (Biovision Tech.), and Photo Fluor II (89 North) for illumination. The images were processed and pseudo-colored with iVision software. For flow cytometry analysis, the wells for each condition were combined and reconstituted in DPBS at 10⁶ cells/mi and stained with 7-Aminoactinomycin (7-AAD) flow cytometry viability dye (Thermo-Fisher Scientific). The cells were run in a 10 color BD FACSCanto with 488 nm excitation filter and 530 nm (clover) and 695 nm (7AAD) detection filters. Each sample was run until approximately 100,000 events were detected. Analysis was done by gating out debris and multi-cell clusters from original side scatter vs forward scatter plot, and then viable and clover-expressing populations were analyzed from the single cell events by applying appropriate compensation of these detector channels and setting thresholds (10³ units) for background emissions. All measurements were performed in the Flow Cytometry Core, Penn State College of Medicine, PA.

Western Blotting Assays

In this set of experiments, HEK and ND7/23 cells were plated at 120,000 cells/well in 6-well plates 24 hours prior to transfection. Linearized or supercoiled clover was loaded onto nanobowls, lipid-encapsulated, and re-dispersed in DPBS:Opti-MEM at 0.5 mg/ml as described above. Each well was then incubated in 1 ml of this solution for 4 hours at 37° C. and then rinsed 3 times in warm DPBS. After 48 hours post-transfection, the cells were trypsinized, dissolved in a lysis buffer containing p-marcaptoethanol. Protein extraction, purification, and collection were performed with the Nucleospin RNA/Protein kit (Macherey-Nagel, Inc.). The protein samples were quantified with the Qubit protein kit (Thermo-Fisher Scientific). The Western blot experiments were then performed with the Wes system (Protein Simple). The microplate was loaded with protein concentrations ranging from 0.025-0.25 μg/μl, primary antibodies and secondary antibodies. The rabbit monoclonal anti-clover (Abcam, Inc.) and anti-vinculin (housekeeping gene, Abcam, Inc.) antibodies were employed at 1:1000 and 1:500, respectively. Protein detection and quantification were performed with the Compass software (Protein Simple).

Transmission Electron Microscopy (TEM)

Between 5 and 10 μl of purified nanobowls (suspended in ethanol or water if lipid-coated) were drop cast on a 400 Cu mesh with Formvar/Carbon Film, Cat: FCF400-Cu (Electron Microscopy Sciences). LNB samples were stained in partially dried state by adding a small drop of 2% urenyl acetate (Electron Microscopy Sciences). The samples were dried by wicking excess solvent with the edge of a soft filter paper and then air-dried at room temperature. Tissue samples were fixed with 2.5% glutaraldehyde and 2% paraformaldehyde (Electron Microscopy Sciences) in 0.1 M phosphate buffer (pH 7.4) and further fixed in 1% osmium tetroxide (Electron Microscopy Sciences) in 0.1 M phosphate buffer (pH 7.4) for 60 minutes. Samples were dehydrated in a graduated ethanol series, acetone, and embedded in LX-112 (Ladd Research). The sections (60 nm) were stained with uranyl acetate and lead citrate (Electron Microscopy Sciences) and viewed in a JEOL JEM 1400 Transmission Electron Microscope (JEOL USA Inc.). All images were taken at 60 kV. All measurements were performed in the Microscopy Imaging Core, Penn State College of Medicine, PA.

Scanning Electron Microscopy (SEM)

After removing the PS core, the nanobowls were purified by centrifugal washing three times and re-dispersed in ethanol. A small volume was applied onto a microscope stub and air dried. The images were acquired with a Zeiss Sigma 500 scanning electron microscope at 2 kV. The images were processed at Nano3 Materials Characterization core facility at University of California San Diego.

Dynamic Light Scattering (DLS)

The nanobowls were purified and reconstituted in water at approximately 50 μg/ml concentrations at various steps. For sizing measurements, the sample dispersions were pipetted onto a disposable PS sizing cuvette (Malvern ZEN0040) and measurements were taken at 90° scattering angle. Zeta potential measurements were obtained with a folded capillary cell (Malvern DTS1070). Both measurements were taken at room temperature in a Zetasizer Nano (Malvern Instruments) at the UC San Diego MRSEC Materials Characterization Facility (MCF).

Co-Transfection of HEK Cells with Opioid Receptors and GIRK1 and GIRK4 Channels

In this set of experiments, HEK cells were plated on glass-bottom 96-well plates at 35,000 cells/well 24 hours prior to the experiment. In one set of experiments, the cells were transfected with 200 μl of 0.5 mg/ml LNBs loaded with yellow fluorescent protein (YFP)-tagged p-opioid receptor (YFP-MOR), GIRK1 and GIRK4 cDNA constructs at a 2:1:1 construct ratio of a total 15 μg. In the second set, the cells were transfected with KOR, GIRK1 and GIRK4 cDNA constructs at a ratio of 1:1:1 with a total of 15 μg cDNA/well. 48 hours post-transfection, the cells were loaded with the voltage sensitive blue dye (FLIPR membrane potential assay kit blue, Molecular Devices) at 37° C. for 30 minutes. Afterwards, fluorescence measurements (540 nm emission) were acquired at 2 second intervals with the FlexStation 3 microplate reader (Molecular Devices). After a stable baseline of 30 seconds was obtained, the specific opioid receptor agonists were applied to each well at different concentrations. Opioids used in this study, for example, fentanyl, oxycodone (p opioid agonists), U-50488, and U-69593 (K opioid agonists) were ordered from Sigma-Aldrich. Control wells received FLIPR buffer only. The maximum percent reduction in fluorescence signal from baseline (signal before t<=30 second) within the 180 second reading time interval (proportional to cell hyperpolarization) was plotted against logarithmic agonist (opioid) concentration range (log M) and fit with Hill equation to obtain the concentration-response plots.

Nanobowl Uptake and cDNA Transfection of Acutely Isolated Rat Sensory Neurons

The animal studies were approved by the Penn State College of Medicine Institutional Animal Care and Use Committee (IACUC). Sprague-Dawley rats were initially anesthetized with CO₂ and rapidly decapitated with a laboratory guillotine. The DRG neurons (L4 and L5) and SCG neurons were isolated as described previously. Both DRG and SCG tissue were then cleared of connective tissue in ice-cold Hanks' balanced salt solution. Thereafter, the tissue was enzymatically dissociated in Earle's balanced salt solution containing 0.6 mg/m 1 collagenase D (Roche Applied Science), 0.4 mg/m 1 trypsin (Worthington Biochemical), and 0.1 mg/m 1 DNase (Sigma-Aldrich) in a shaking water bath at 35° C. for 60 minutes. Thereafter, the neurons were dispersed by vigorous shaking, centrifuged twice for 6 minutes at 44×g, and resuspended in MEM (Thermo-Fisher Scientific) supplemented with 10% fetal bovine serum, 1% penicillin-streptomycin, and 1% glutamine (Thermo-Fisher Scientific). Finally, the neurons were plated onto 35 mm poly-L-lysine-coated dishes and stored in a humidified incubator supplied with 5% CO₂/95% air at 37° C. For internalization experiments, the neurons were exposed to nanobowls (30 μg/ml), mixed in DPBS:Opti-MEM for 4 hours, rinsed in warm DMEM, and fixed as per TEM fixation protocols. For LNB internalization experiments, LNBs (0.5 mg/ml) were added as above and applied to the neurons for 4 and 24 hours prior to fixation as per TEM protocols. For transfection of DRG neurons, the dissociated cells were incubated for 4 hours with 0.5 mg/ml LNB that were loaded with 10 μg/mg supercoiled clover cDNA. The neurons were fixed in 4% paraformaldehyde (PFA) 48 hours post-transfection. Afterwards, phase contrast and fluorescence images were acquired as described above. In another set of experiments, the non-dissociated DRG tissue (L4 and L5) were placed in a 96-well plate and incubated initially in DMEM with 3% DMSO in order to dissociate the meningeal layer at 37° C. for 30 minutes. Thereafter, the tissue was incubated in 300 μl LNB (1 mg/ml) loaded with 32 μg/mg linearized tdT at 37° C. for 6 hours. For internalization study, amine-nanobowls were conjugated to Cy3-NHS (Lumiprobe Inc.) in DMSO, washed, dried, coated with DOPE/DOTAP as described before, and finally re-dispersed in 1:1 OMEM:DPBS at 1 mg/ml and 0.5 mg/m 1 concentrations, respectively. The tissue was gently rinsed 3 times in warm DMEM and then incubated for 72 hours at 37° C. in DMEM supplemented with growth factor (15 ng/ml ciliary derived growth factor, 15 ng/ml nerve growth factor and 6 ng/ml glial derived neurotrophic factor). After the incubation period, the tissue was dissociated employing the protocol described above and plated on poly-L-lysine-coated 35 mm tissue culture dish for fluorescence imaging. All images were pseudo-colored in accordance with the appropriate filters used for the fluorescence channel.

### Results

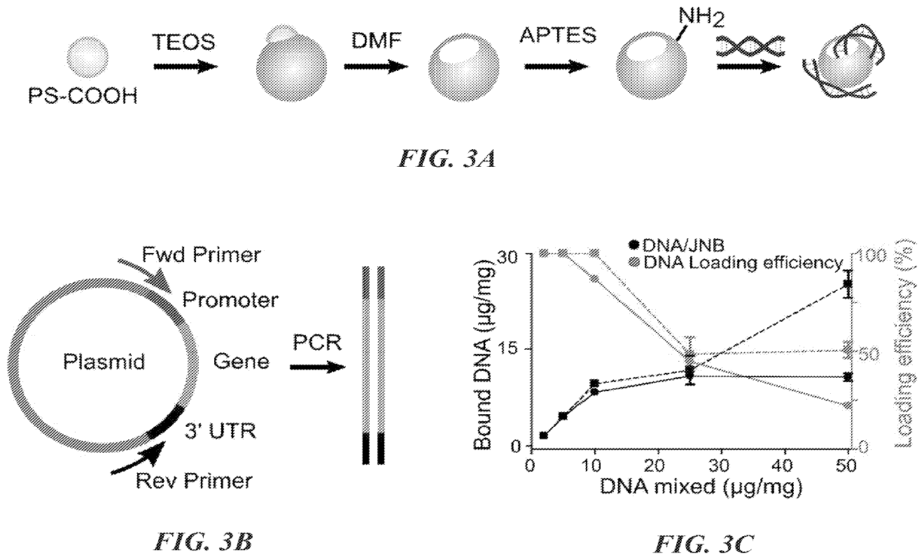

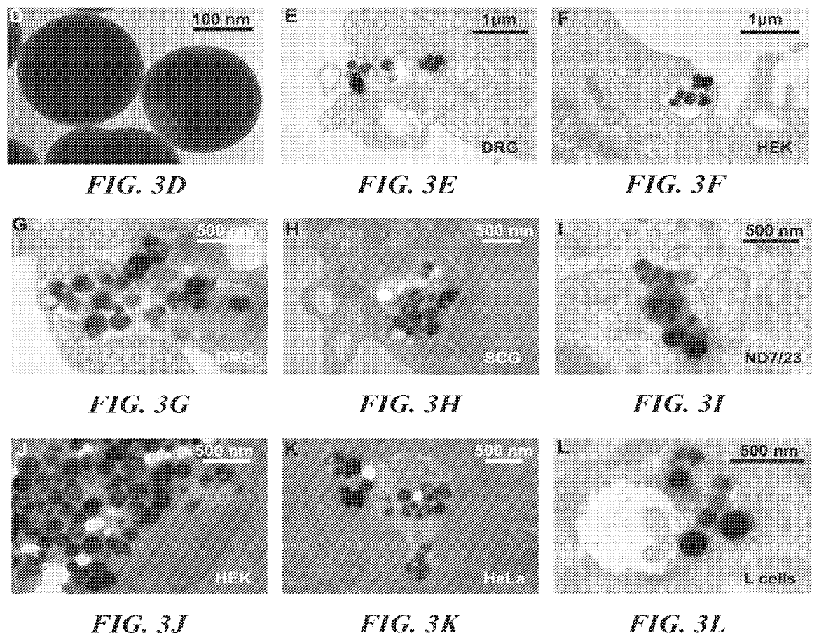

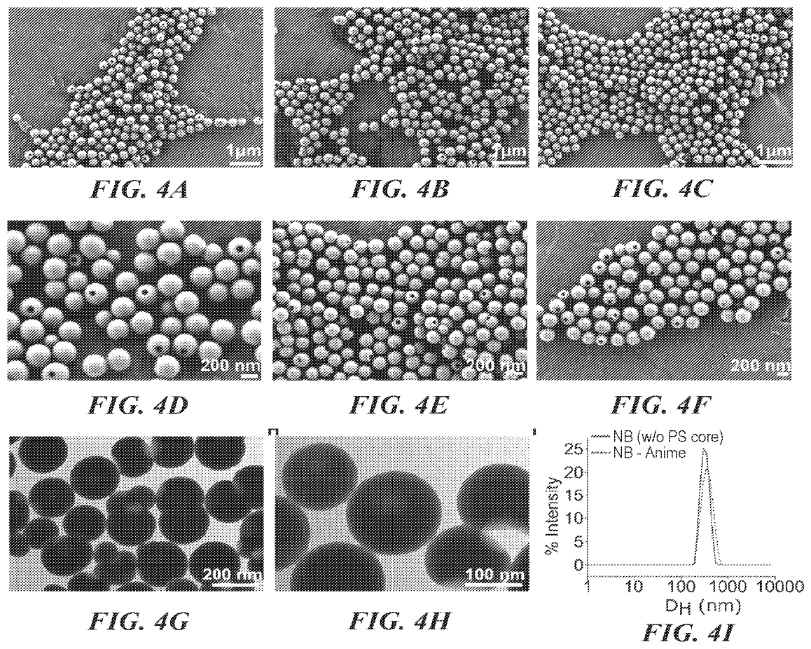

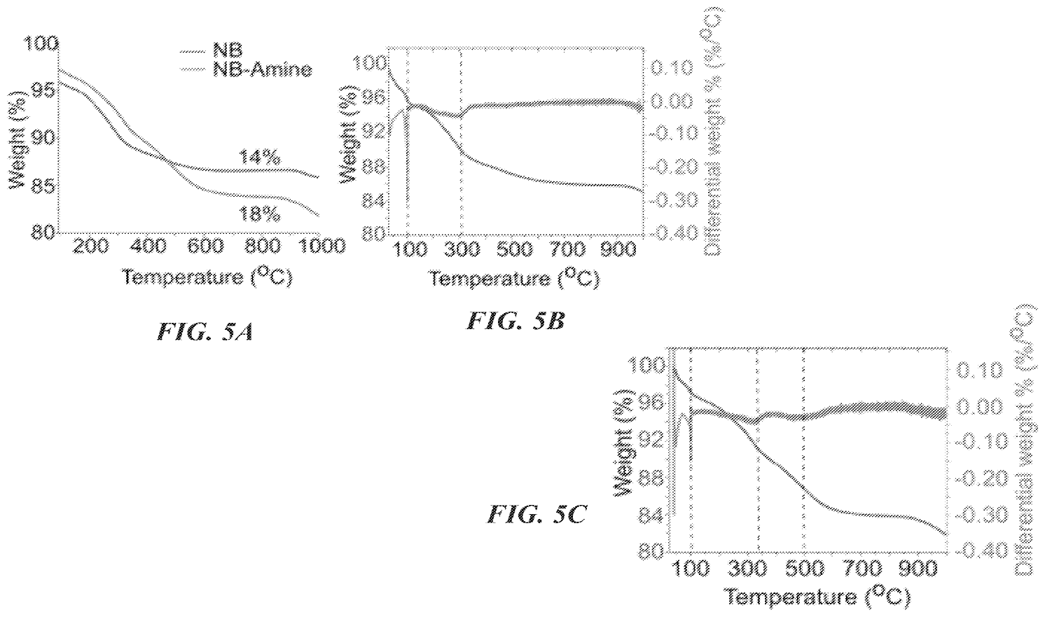

FIG. 3A is a schematic illustrating the synthesis of the nanobowls designed to transfect cells with cDNA. Silica nanobowls were synthesized by polymerization of TEOS around a 100 nm PS template. After removal of the PS template, the nanobowl surface was functionalized with amine groups by silanization with APTES. The representative TEM micrograph in FIG. 3D shows DMF-washed nanobowls with a hydrodynamic size distribution of approximately 314.1±6.6 nm (PDI=0.032±0.025) and a mean engineered cavity of about 65±8 nm (FIGS. 4A-4I). APTES loading was confirmed by measuring the mass loss due to incremental heating in a nitrogen environment from 100-1,000° C. in thermogravimetric analysis (TGA). The TGA results (FIGS. 4A-4I) indicate that the bare nanobowls exhibited a lower overall mass loss percentage than the APTES silanized nanobowls over the same temperature range. We measured a loading of approximately 210 μmol/g APTES (Table 1). Following APTES functionalization, the zeta potential, measured in water, of the nanobowls changed from −34.5±0.6 mV to 36.8±0.8 mV (Table 1).

TABLE 1
Characterization of size and surface charge of nanobowls

| | Hydrodynamic | Polydispersity | Zeta |
|---|---|---|---|
| | diameter | Index | Potential |
| Sample | (nm) | (PDI) | (mV) |
| Nanobowls | 314.1 ± 6.6 | 0.032 ± 0.025 | −34.5 ± 0.6 |
| (no PS core) | | | |
| Nanobowls (Amine- | 343.4 ± 5.3 | 0.114 ± 0.037 | +36.8 ± 0.8 |
| functionalized) | | | |
| Nanobowls (Amine- | 370.1 ± 11.1 | 0.177 ± 0.033 | −49.0 ± 2.2 |
| functionalized, 10 μg/mg | | | |
| plasmid loaded) | | | |

The nanobowls were purified in each case as described in methods and re-dispersed at approximately 50 μg/ml concentration in deionized water and measurements were acquired in triplicate. Error values reported are standard deviations for n = 3 repeats.

TABLE 2
TGA weight losses categorized in nanobowls
and amine-functionalized nanobowls

| | | Nanobowl | | Nanobowl-Amine | |
|---|---|---|---|---|---|
| | Materials | (% Weight loss) | | (% Weight Loss) | |
| Temperature | contributing | Aver- | Std. | Aver- | Std. |
| ranges | to weight loss | age | Dev | age | Dev |
| Weight loss % | Water | 4.25 | 0.03 | 2.88 | 0.02 |
| <= 100° C. | | | | | |
| Weight loss % | Solvents | 5.66 | 0.25 | 5.60 | 0.66 |
| >100° C. and | | | | | |
| <300° C. | | | | | |
| Weight loss % | Other bound | 5.00 | 0.21 | 9.64 | 0.64 |
| >300° C. | organics | | | | |

As shown in Table 2, the weight loss beyond 300° C. in amine-functionalized nanobowls was first background subtracted from nanobowl only sample. 4.64%±0.67% net weight loss was determined to attribute to APTES based surface functionalization of nanobowls, with average and standard deviations from duplicate measurements (n=2). This mass loss was calculated to be equivalent to 209.6 μmol g−1 APTES (Mw=221.4 g/mol) loading. Alternatively, by considering mass loss of nanobowl-amine in 300-500° C. and 500-1000° C. regimes separately, and background subtracting from DMF-washed nanobowls only, 5.21±0.91% total mass loss is calculated which results in 236.7 μmoles g−1. Assuming 1 mole of amine groups come from decomposition of 1 mole of APTES, we report here 209.6-236.7 μmoles g−1 amine loading on the nanobowls.

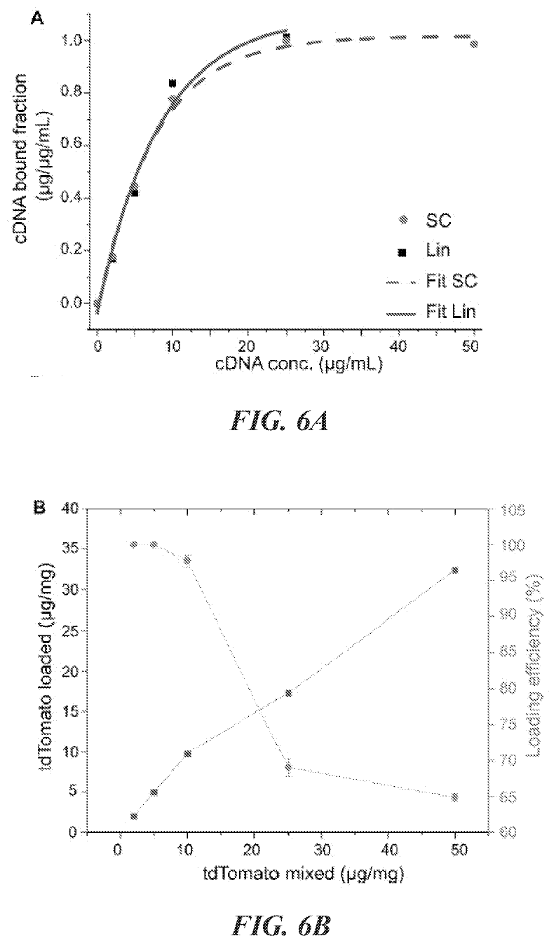

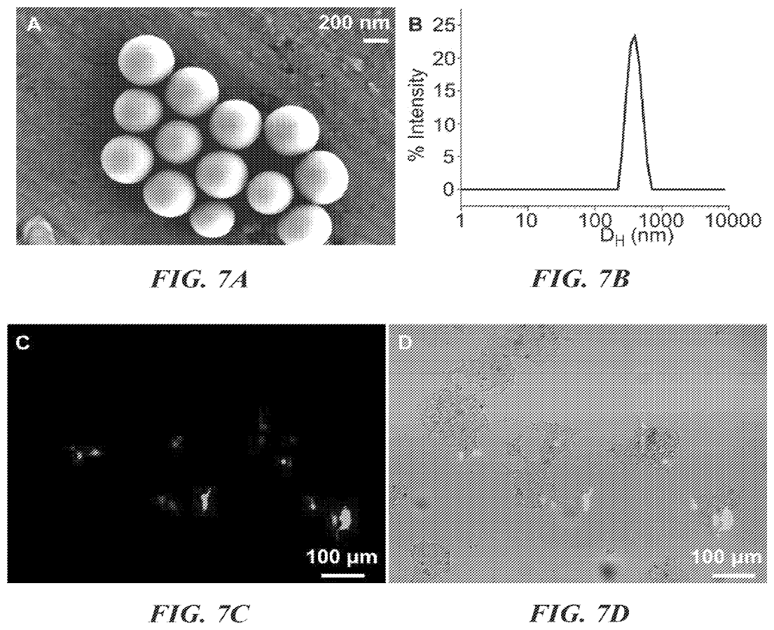

The cDNA constructs employed for transfecting in this study were either linearized (i.e., vector-less) or supercoiled. To synthesize linearized cDNA, the coding region of a supercoiled, vectored cDNA template was amplified with primers that were specific for the CMV promoter region (forward primer) and the polyA tail region (reverse primer) of the template plasmid by PCR (FIG. 3B). FIG. 3C is a plot that shows amine-coated nanobowls can load both linearized and supercoiled cDNA constructs. Unlike linearized cDNA, the adsorption profile of the supercoiled construct showed an exponential trajectory with a saturation plateau. The maximum bound cDNA achieved was 11 μg/mg nanobowl for supercoiled (black circles) and 25 μg/mg linearized (black squares) clover cDNA. FIG. 3C also indicates that the loading efficiency decreased with increasing cDNA concentrations. At the highest DNA tested (50 μg/mg), the loading efficiencies for linearized (red circles) and supercoiled (red squares) cDNA were 22% and 50%, respectively. The profile observed for supercoiled cDNA suggests that nanobowls possess a monolayer saturation adsorption capacity. The K_{D} value from the exponential fit was 5.5 μg/ml for supercoiled cDNA. On the other hand, linearized cDNA constructs exhibited a similar binding profile up to 25 μg/ml after which there is a linear increase in binding without reaching a saturation point with the highest amount of cDNA tested (FIG. 6A). Therefore, it was not possible to fit the entire range of the linearized cDNA binding data with a single exponential equation. However, fitting the data to an exponential equation up to 25 μg/ml, the K_{D} obtained was 5.9 μg/ml (R²=0.98) (FIG. 6A). APTES-modified Stöber silica nanoparticles, without PS template-induced cavities, exhibited a comparable size (DH=415.3±21.9 nm; PDI 0.207±0.043 in water) as the nanobowls. Further, these nanoparticles showed a loading of approximately 9 μg/mg for 25 μg/ml added supercoiled plasm id clover cDNA (FIGS. 7A-7D).

We next examined whether increasing the size of the linearized DNA would alter the loading curve. In this set of experiments, we employed the cDNA construct coding for the fluorescent protein, tdTomato (tdT). Our results (FIG. 6B) indicate that the tdT binding profile was steeper than that observed for clover (FIG. 3C). The maximum loading capacity observed at 50 μg/mg loading was 32 μg/mg, slightly greater than the linearized clover (25 μg/mg). However, based on the higher molecular weight of linearized tdT (2.4 kDa) than clover (1.7 kDa), we determined that a comparable cDNA copy number was adsorbed on the nanobowl surface per mg (New England Biolabs NEBiocalculator™: tdT copy number=1.3×10¹³ and clover copy number=1.4×10¹³).

In the next set of experiments, acutely dissociated rat peripheral neurons (SCG and DRG) and ND7/23, HEK, HeLa, and L-cells were transfected with nanobowls (30-100 μg/ml) loaded for 4 hours. We found that nanobowl concentrations greater than 100 μg/ml would lead to high cell death. The TEM images in FIGS. 3E-3L show that the nanobowls (30 μg/ml) were internalized within 4 hours by all cell types tested. The nanobowls in FIGS. 3E-3F appear to be endocytosed via a phagocytotic mechanism.

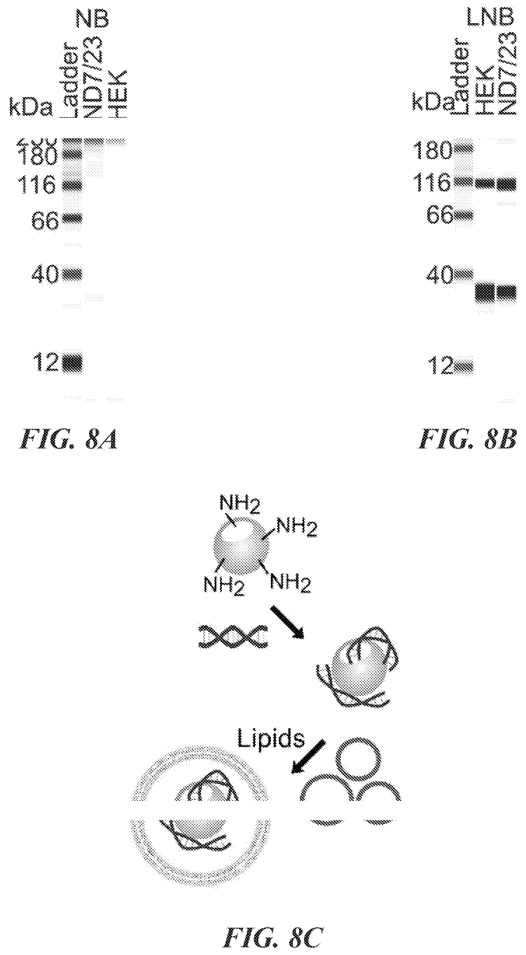

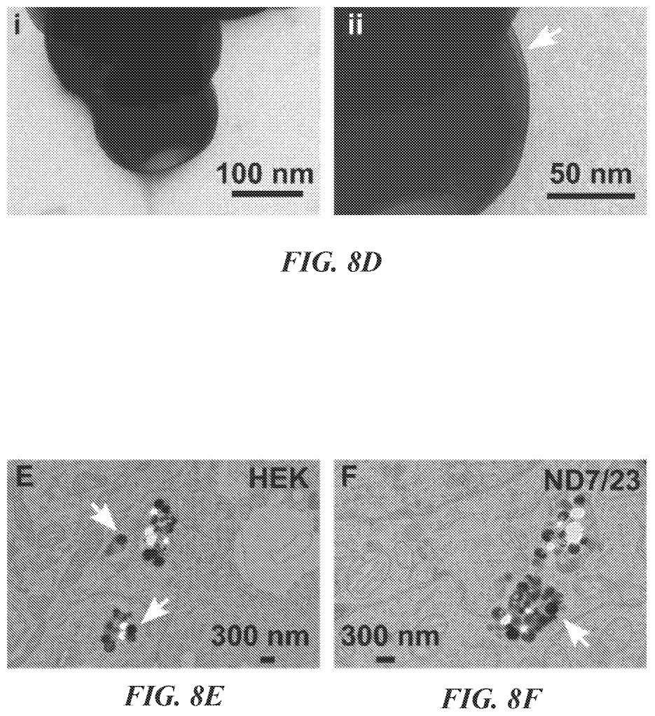

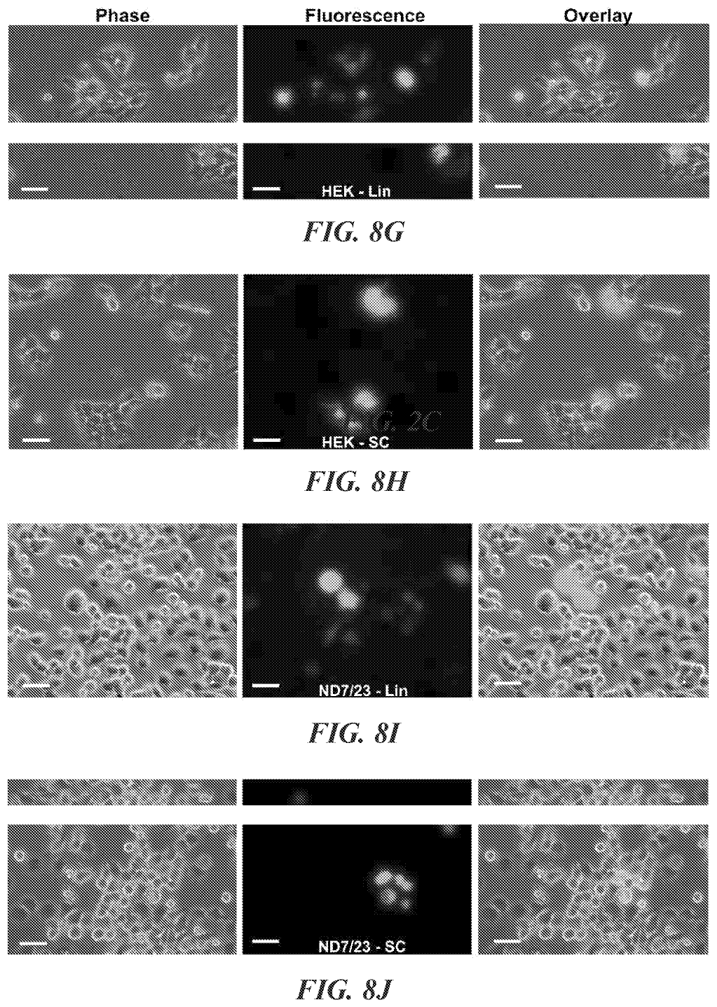

Although the clover cDNA-loaded nanobowls were internalized by all cell types, clover fluorescence was not observed up to 72 hours post-transfection with nanobowl concentrations up to 0.5 mg/ml. To confirm the lack of clover expression, HEK and ND7/23 cells were transfected with 0.1 mg nanobowls carrying 2 μg/mg clover cDNA for 4 hours and cell protein was isolated 72 hours post-transfection. Western blotting assays were employed to detect clover expression and the blot shown in FIG. 8A indicates neither cell type was successfully transfected.

The lack of clover expression suggested that the internalized nanobowls were trapped within endocytic vesicles and unable to release the cDNA. Consequently, the nanobowls were next coated with the lipids DOPE and DOTAP (1:1 molar ratio), which have been reported to act as “helper agents” in other transfection systems. The TEM micrographs shown in FIG. 8D indicate that the LNBs have a lipid coating of approximately 5 nm. HEK and ND7/23 cells were transfected for 4 hours with LNBs (0.5 mg/ml) carrying linear or supercoiled clover cDNA constructs (10 μg/mg). Clover protein expression levels were then determined with Western blotting assays. FIG. 8B shows that, unlike nanobowls, LNBs loaded with supercoiled clover cDNA resulted in protein expression in both cell lines. Furthermore, the TEM micrographs shown in FIGS. 8E-8F indicate that LNBs were internalized in HEK (FIG. 8E) and ND7/23 (FIG. 8F) cells within 4 hours of incubation, and some nanobowl clusters were found in the cytoplasm that seem to have escaped endosomal entrapment (white arrows, FIGS. 8E-8F). The fluorescence images acquired 48 hours post-transfection with LNBs also indicate that clover expression in both HEK (FIGS. 8G-8H) and ND7/23 cells (FIGS. 8I-8J) was successful with either linear (FIGS. 8G, 8I) or supercoiled (FIGS. 8H, 8J) cDNA construct.

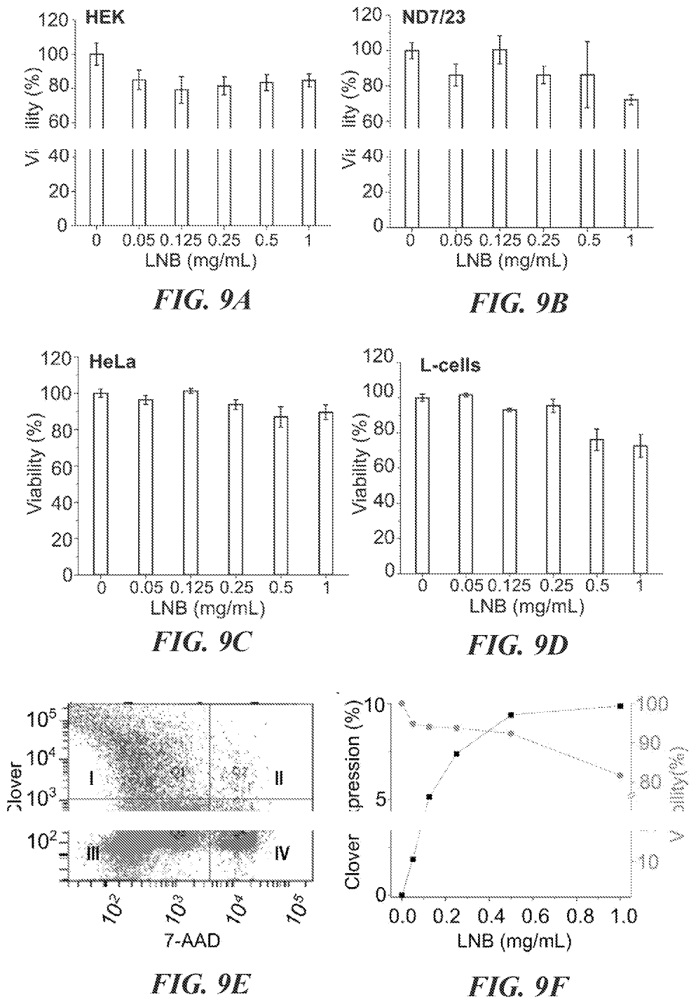

In the next set of experiments, MTT assays were performed to determine LNB (0.05-1 mg/ml) toxicity (without cDNA payload) in HEK, HeLa, ND7/23, and L-cells following transfection for 4 hours. The plots shown in FIGS. 9A-9D indicate that at the highest LNB concentration tested (1 mg/ml), toxicity was highest (˜28%) for both ND7/23 and L-cells when compared to both HEK and HeLa cells. However, HEK cells showed a consistent toxicity (˜16%) for all concentrations tested (FIG. 9A). These results suggest that for these cell lines, a 4 hour transfection period with LNBs will result in toxicity ranging from 10% to 28% at the highest concentration (1 mg/ml) tested, and lower toxic effects with LNBs at 0.125 mg/ml.

We next assessed the optimal LNB concentration (0.05-1 mg/ml) necessary for transfection, while maintaining a constant cDNA loading (10 μg cDNA/mg). HEK cells were transfected with linearized clover cDNA and flow cytometry assays were performed 48 hours post-LNB incubation to ascertain both the transfection efficiency (i.e., clover expression) and cell viability. The scatter plot in FIG. 9E depicts clover expression for single cell population (y-axis) as a function of cell viability (x-axis). The transfection efficiency for single cells (percentage of cells counted from quadrants I and II, FIG. 9E) ranged from 2% to 10% as the concentration of LNB increased, and is illustrated in FIG. 9F. The representative plot (in black) indicates that a plateau was reached at concentrations greater than 0.5 mg/ml. This suggests that higher LNB concentrations approach saturation of possible expression in HEK cells. Under our conditions, saturation in expression was reached at 10% for clover, calculated with a Hill fit of the data, slope of 0.12 and non-linear regression of 0.99. We also observed that the cell viability, measured by cell populations with negligible 7AAD emission (percentage of cells counted from quadrant I and III, FIG. 9E), increased from 82% at 0.05 mg/ml to 95% at 1 mg/ml (red trace, FIG. 9F). These results suggest there is a trade-off when LNBs are employed as transfection agents. That is, when high LNB concentrations are used, protein expression levels are higher with a concomitant cell death. To determine the optimal LNB concentration necessary for the highest transfection efficiency, we determined that at 0.5 mg/ml, the cell viability is 92% and clover expression levels reach approximately 10%. This value is close to the saturation level of expression while still maintaining >90% cell viability. At this concentration, we also observed approximately 1% dead clover-expressing cells (Table 3). Thus, 0.5 mg/ml is the optimum LNB concentration necessary to ensure maximum possible expression with greater than 90% cell viability. It should be noted that analysis of the flow cytometry data was performed for single cell populations, while cell clusters containing both clover- and non-clover-expressing populations were excluded from the analysis (Table 3).

TABLE 3
LNB concentration dependent flow cytometry parameters

| LNB | % Events | % Clover |
|---|---|---|
| conc. | from | Positive, |
| (mg/ml) | clusters | dead cells |
| 0.000 | 23 | N/A |
| 0.050 | 37 | 0.1 |
| 0.125 | 33 | 0.6 |
| 0.250 | 33 | 0.6 |
| 0.500 | 28 | 0.9 |
| 1.000 | 29 | 1.4 |

As shown in Table 3, cluster population percentages are reported above for all LNB concentrations tested with transfected HEK cells in flow cytometry assay. These events were gated out and were excluded from percent GFP positive population analysis. Among the clover-expressing cells, further live/dead gating in the 7AAD channel (quadrant II in FIG. 9E) was applied to determine percentage of dead clover-expressing cells at various LNB transfection concentrations shown above.

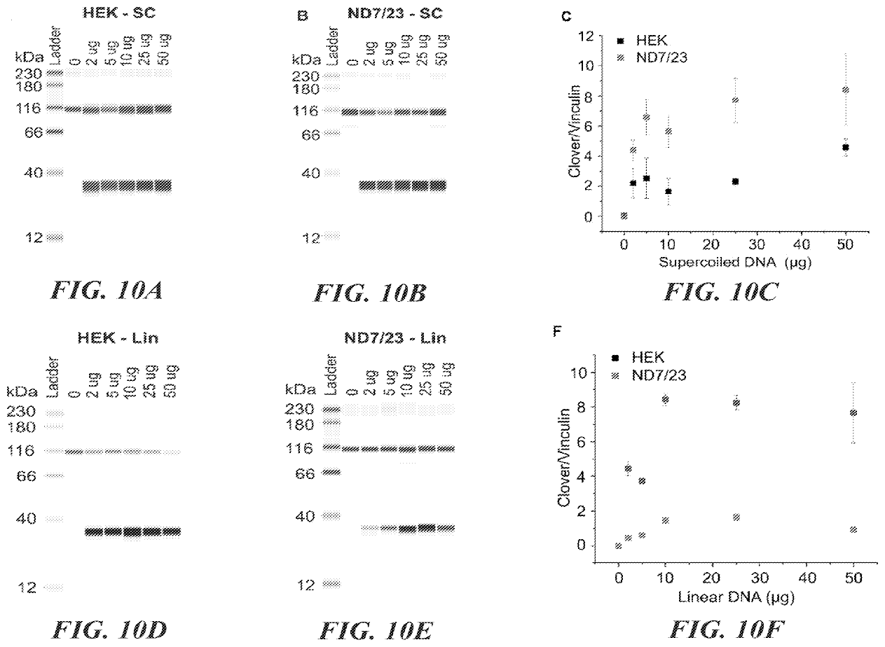

We next determined the transfection efficiency in both HEK and ND7/23 cells with varying loads (0-50 μg/mg) of clover DNA (linear and supercoiled) with a constant LNB concentration (0.5 mg/ml). Clover expression was quantified employing Western blotting assays 48 hours post-transfection with LNB (FIGS. 10A-10F). The results indicate that clover expression, determined by the clover/vinculin ratio, was higher in ND7/23 cells for all cDNA loads tested (FIG. 10C). On the other hand, HEK cells exhibited greater clover expression when transfected with LNBs loaded with linear cDNA (FIG. 10F). Although clover transfection can be obtained with LNBs loaded with either supercoiled or linearized cDNA, the relative expression levels when transfected with the LNB system are cell type dependent.

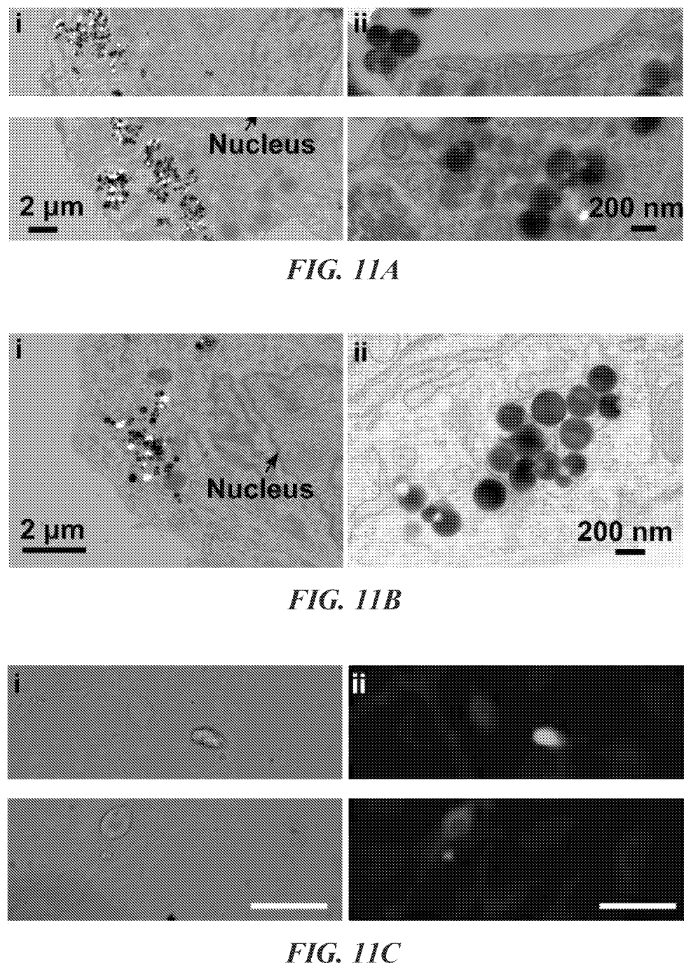

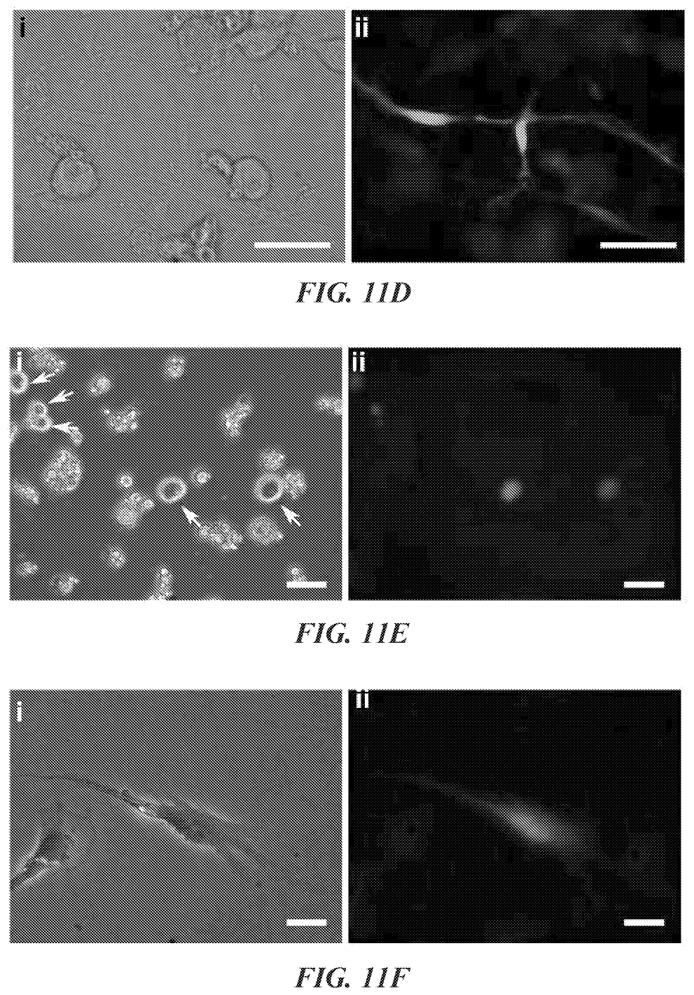

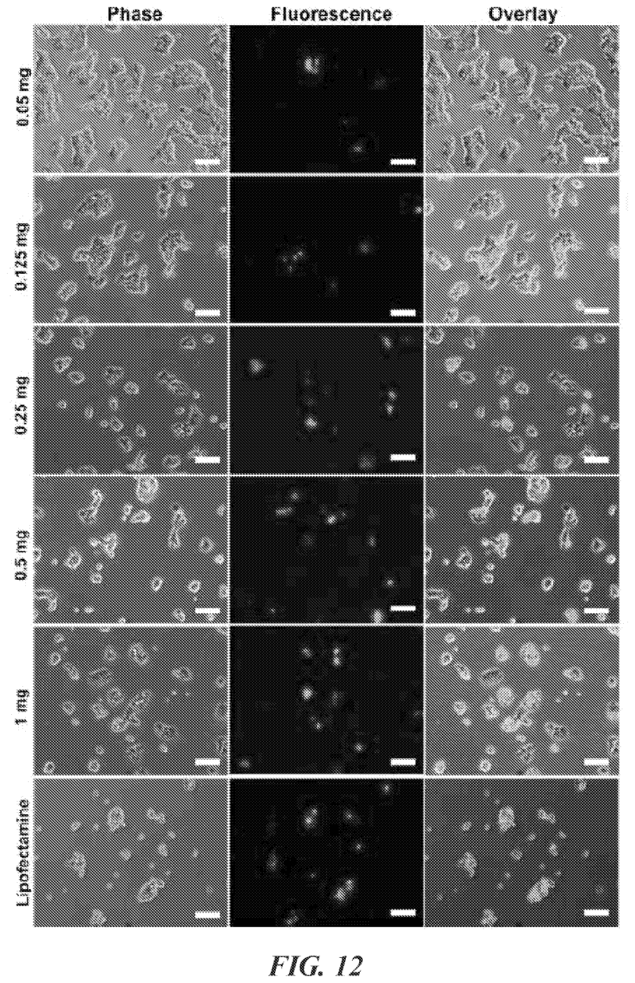

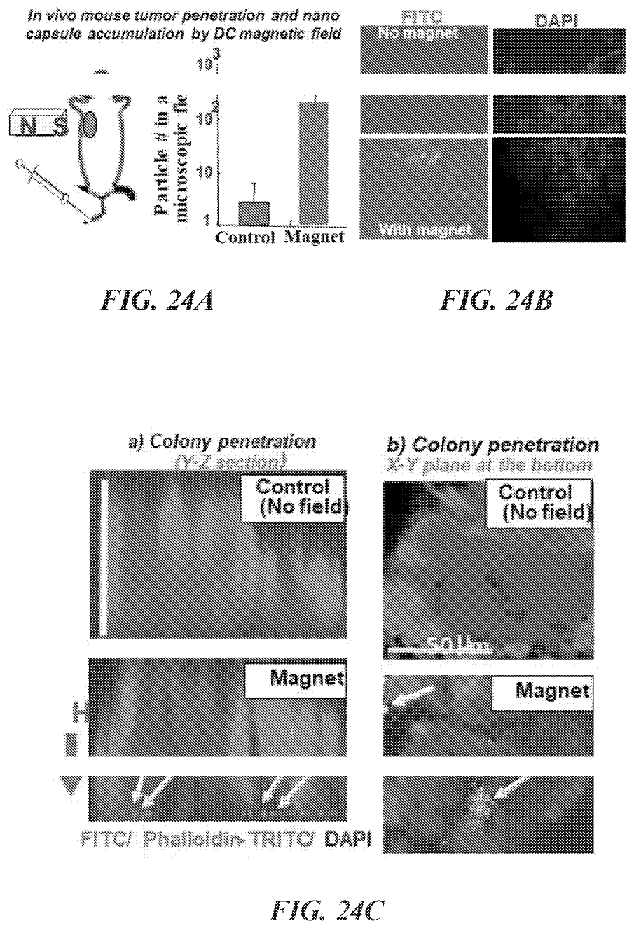

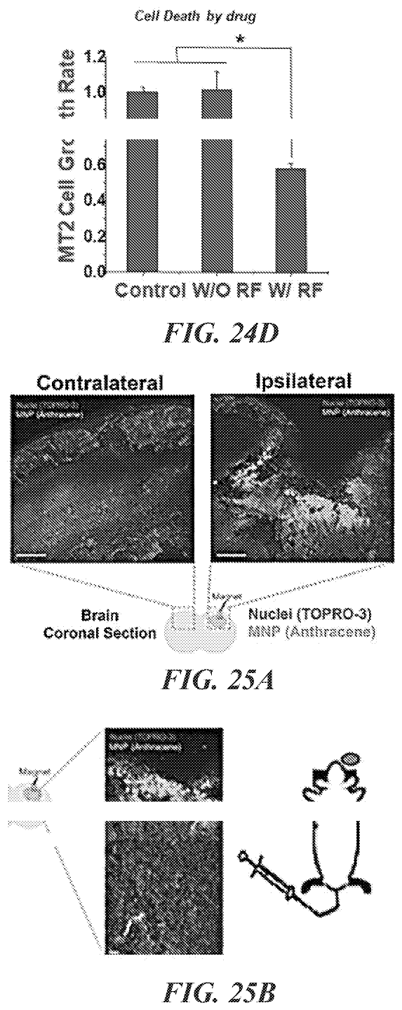

We next examined whether clover cDNA-loaded LNBs (0.5 mg/ml) could be employed to transfect acutely dissociated rat DRG neurons. The LNBs were loaded with supercoiled clover cDNA (10 μg/mg). The TEM micrographs acquired 4 hours (FIG. 11A) and 24 hours (FIG. 11B) post-transfection show that LNBs remain internalized within the cytoplasm. The micrographs also depict several LNB clusters that are free of vesicular encapsulation post-endocytosis. FIGS. 11C-11D are phase and fluorescence images of acutely dissociated DRG tissue. The images show that both neurons (FIG. 11C) and glial cells (FIG. 11D) expressed clover within 48 hours of in vitro transfection post-dissociation. Whether DRG tissue, prior to enzymatic dissociation, could be transfected with LNBs was also tested. In this set of experiments, cDNA coding for the fluorescent protein tdT was employed. The DRG tissue was incubated for 6 hours with LNBs (1 mg/ml) and loaded with linearized cDNA (50 μg/mg). The DRG tissue was dissociated 72 hours post-transfection and the neurons were then plated in 35 mm dishes. Similar to the results described above, the fluorescence images shown in FIGS. 11E-11F indicate that DRG neurons were successfully transfected with tdT cDNA-containing LNBs.

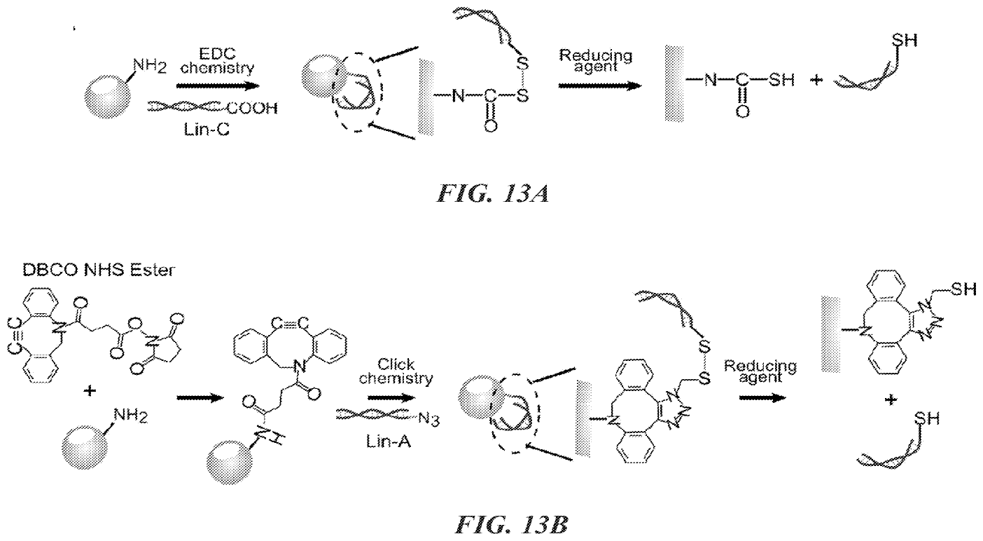

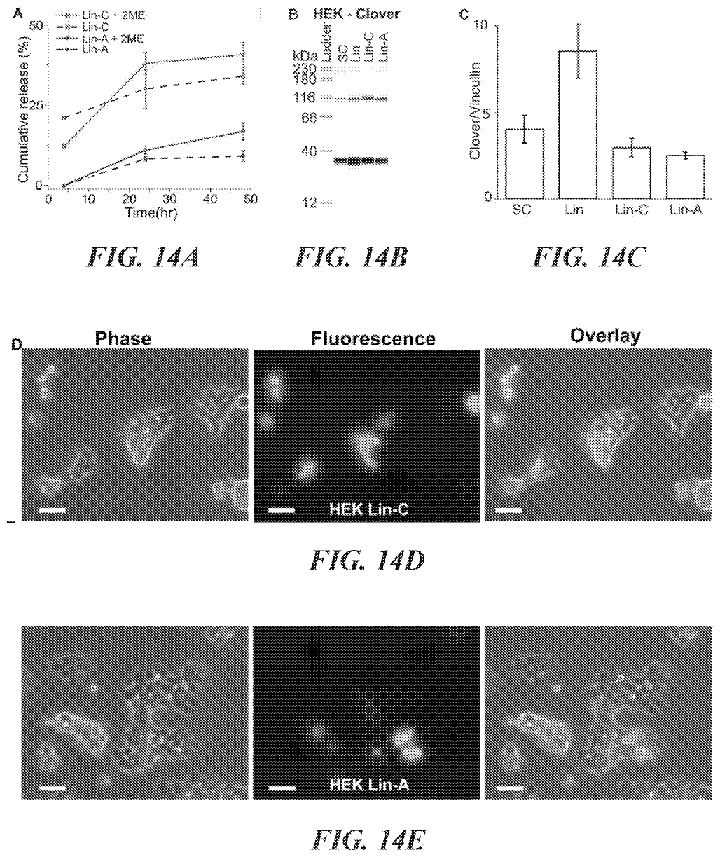

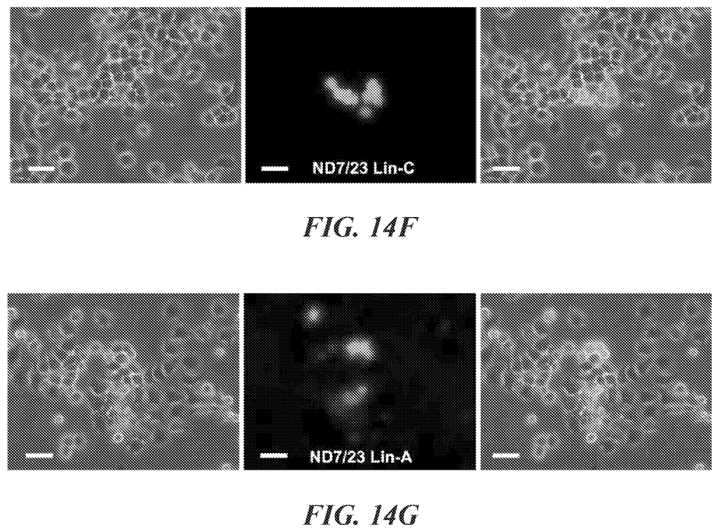

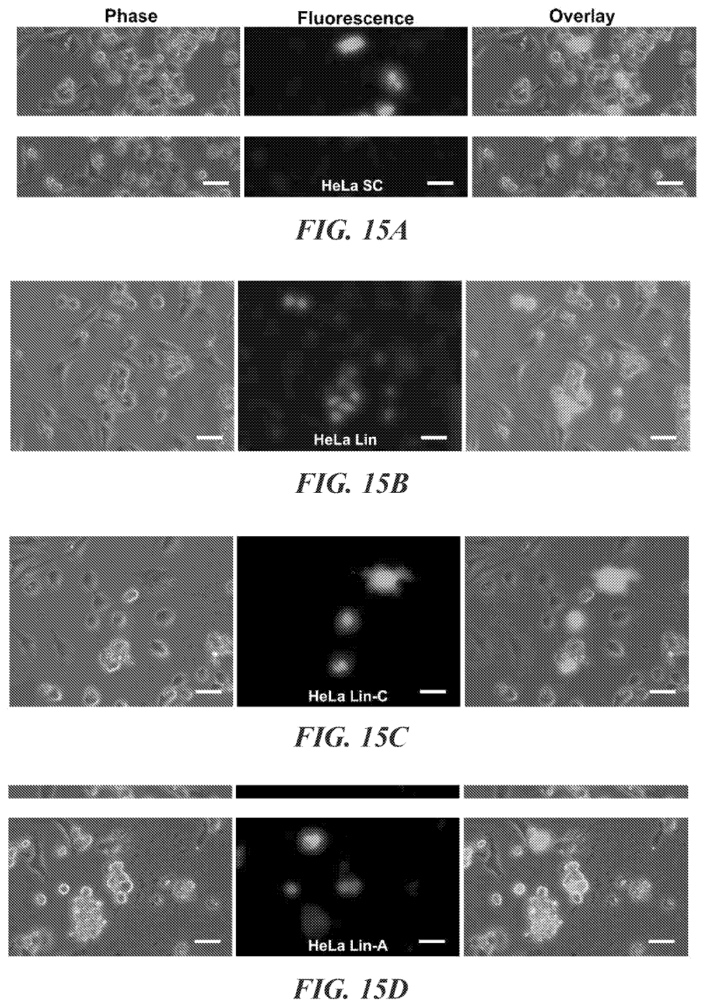

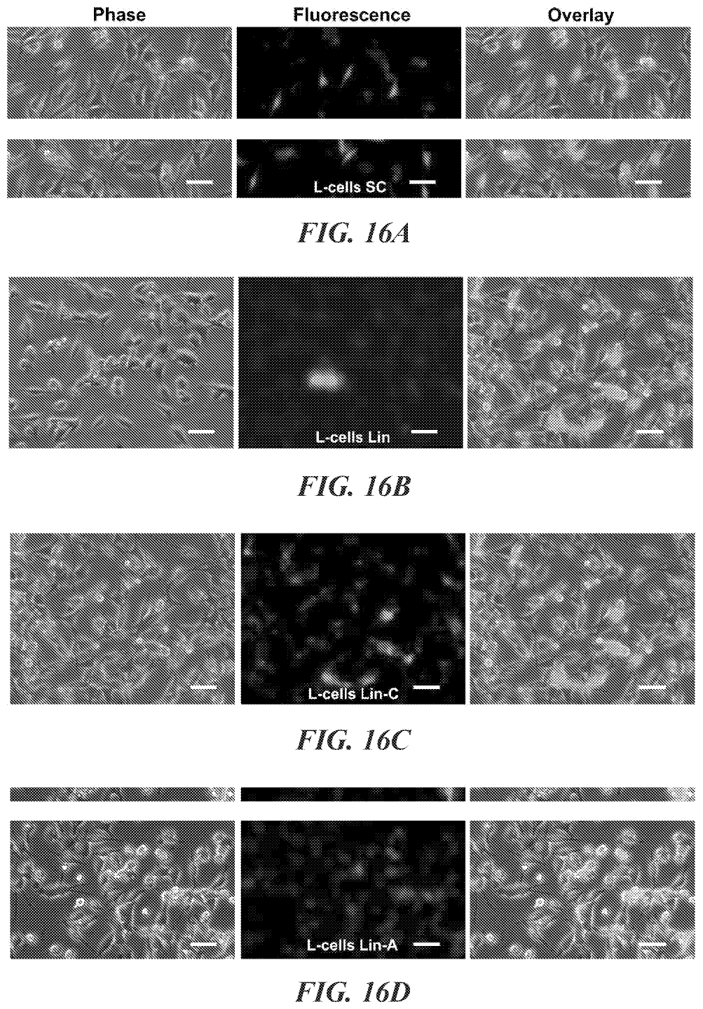

In this set of experiments, we characterized the controlled release of linearized clover DNA, containing cleavable disulfide groups, chemisorbed on the nanobowls. We employed PCR forward primers (see Experimental Procedures) that would allow for the incorporation of a disulfide bond linked to a terminating carboxyl (Lin-C) or azide (Lin-A) group. The chemisorption to nanobowls was accomplished employing either EDC linker-based conjugation chemistry (Lin-C) or azido-DBCO click chemistry (FIGS. 13A-13B). Complete adsorption of DNA (10 μg/mg) was observed by 48 hours. However, the release properties for both chemistries were different. That is, in the presence of a reducing agent, the total release of Lin-C-nanobowls (red symbols) and Lin-A-nanobowls (black symbols) were 41% and 17%, respectively (FIG. 14A). However, we also detected nonspecific release in the absence of a reducing agent for both chemistry types. They were 34% and 9% for Lin-C-nanobowl and Lin-A-nanobowl, respectively (FIG. 14A). These observations suggest that the final adsorption on the nanobowls is caused by both specific chemisorption and nonspecific physisorption. Although Lin-C-nanobowl exhibited a higher overall DNA release within 48 hours, 16% of the DNA released was a result of disulfide bond cleavage. On the other hand, 45% of the DNA released from Lin-A-nanobowl resulted from cleavage of the disulfide bond. An overall release of less than 50% of the total nanobowl-bound cDNA was observed in the in vitro release assay with either chemistry (FIG. 14A). HEK and ND7/223 cells were then transfected with either Lin-C-LNB or Lin-A-LNB clover cDNA and clover expression was examined 48 hours post-transfection by microscopy. FIGS. 14A-14G show images of HEK (FIGS. 14D-14E) and ND7/23 (FIGS. 14F-14G) cells transfected with linearized cDNA. The fluorescence images indicate that clover expression occurred in both cell types with either cDNA construct. Similar results were obtained with HeLa and L-cells (FIGS. 15A-15D, 16A-16D).

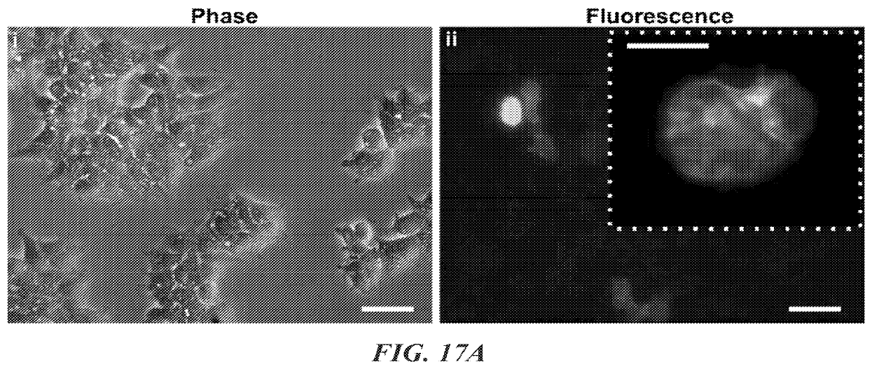

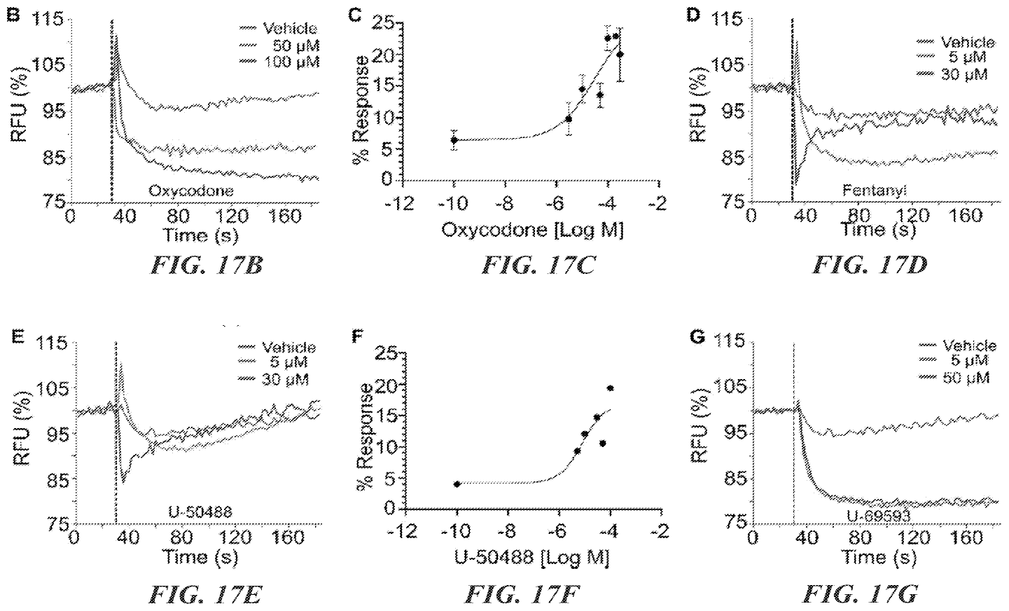

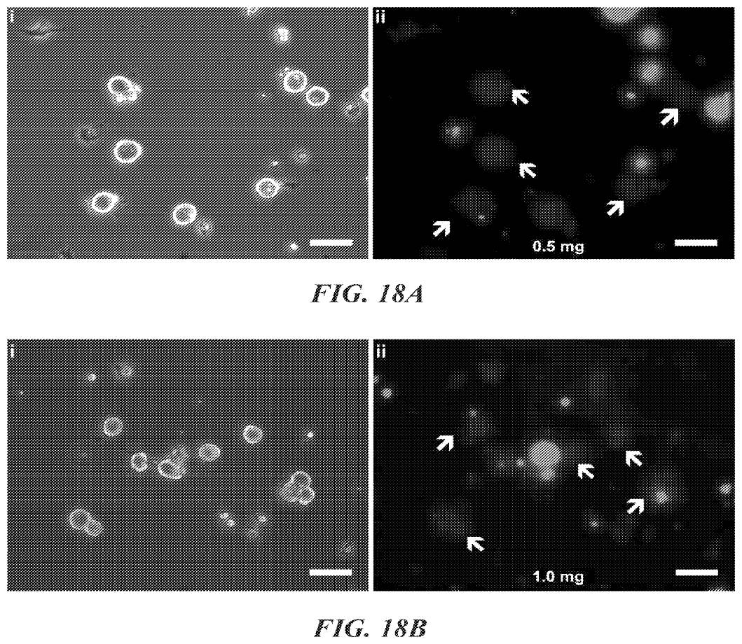

In the final set of experiments, we examined whether LNBs coated with multiple cDNA constructs could be employed to obtain simultaneous protein expression. HEK cells were transfected with LNBs loaded with three cDNA constructs coding for the yellow fluorescent protein-tagged μ opioid receptor (YFP-MOR), G-protein coupled inwardly-rectifying K⁺ channels 1 (GIRK1) and GIRK4. HEK cells do not naturally express these proteins and, thus, provide a suitable null background. Stimulation of MOR leads to G-protein-mediated opening of GIRK1/4 channel dimers and results in cellular hyperpolarization. FIG. 17A shows phase and fluorescent images of HEK cells transfected with the cDNA constructs 48 hours post-transfection.

We optimized a fast, high throughput assay employing a microplate reader with a robotic system which added agents to the 96-well plate seeded with the transfected HEK cells. We employed the FLIPR membrane potential assay kit to measure the opioid-mediated stimulation of MOR and activation of GIRK1 and GIRK4 channels, leading to cellular hyperpolarization. FIG. 17B shows the fluorescence signals of 3 individual wells with HEK cells expressing YFP-MOR, GIRK1, and GIRK4 before and following addition of vehicle (black trace), 50 μM (green trace), and 100 μM (blue trace) oxycodone, a high affinity MOR agonist. Following a 30 second stable baseline, vehicle or agonist application was performed. It can be observed that vehicle did not exert an overt effect on the fluorescence signal, while oxycodone at either concentration caused a decrease in fluorescence, indicative of cell hyperpolarization (i.e., stimulation of GIRK channels). FIG. 17C shows the concentration-response relationship of the oxycodone-mediated decrease in fluorescence. A fit of the data with the Hill equation resulted in an EC₅₀ value of 32.6 μM for oxycodone. We also tested the effect of fentanyl, another high affinity MOR agonist, on the membrane potential of transfected HEK cells (FIG. 17D). It can be observed that the decrease in fluorescence was dose-dependent for 5 μM (green trace) and 30 μM (blue trace) fentanyl. The former caused a 17.1% decrease, while application of the latter led to a 21.2% decrease (FIG. 17D).

We next tested whether another opioid receptor subtype, KOR, could be co-transfected similarly with GIRK1 and GIRK4 in HEK cells. The fluorescence signals shown in FIG. 17E depict the changes in membrane potential of HEK cells co-expressing the three cDNA constructs following exposure to the high affinity KOR agonist, U-50488. Similar to the changes observed with MOR stimulation, application of 5 μM (green) and 30 μM (blue) U-50488 resulted in a dose-dependent cellular hyperpolarization. The representative U-50488 concentration-response relationship is depicted in FIG. 17F. After the data was fit to the Hill equation, the calculated EC₅₀ for U-50488 was 7.95 μM. Thereafter, we examined the effect of a second KOR agonist, U-69593 (FIG. 17G). Application of either 5 μM or 50 μM U-69593 lead to a 21% in fluorescence (FIG. 17G).

### Discussion

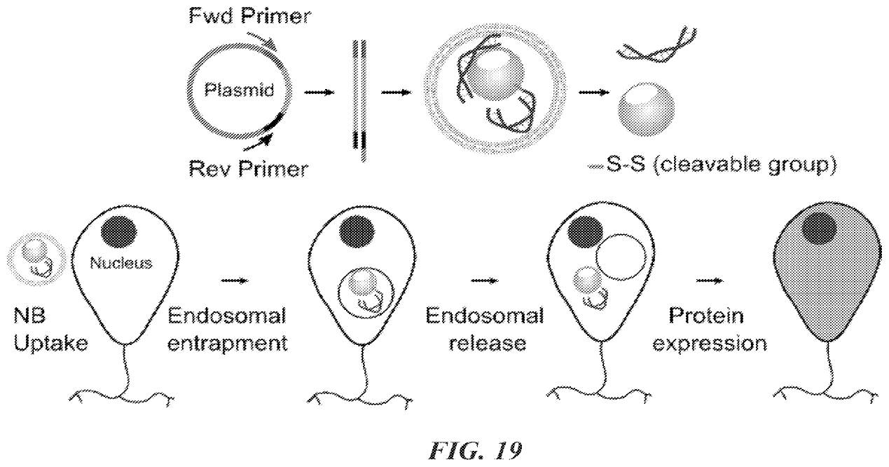

Vesicle entrapment has been reported as one of the major barriers to delivery of DNA or drugs inside the cytoplasm for a number of non-viral transfection agents, including multifunctional silica nanoparticles. Among the strategies to overcome endosomal entrapment, the use of “helper” lipids to coat nanobowls was performed in this study (see FIG. 19). The lipid DOPE was chosen due to its ability to form inverted hexagonal structures that can easily fuse with cellular lipid bilayers and vesicular compartments and facilitate the release of loaded DNA from the nanobowls. DOTAP was chosen as a cationic lipid to stabilize the lipid bilayer on the DNA-loaded nanobowl surface and provide colloidal stability in the media. Previously, it was shown that mesoporous silica nanobowls overcame endosomal entrapment by loading with the endosomolytic compound, chloroquine. However, the drawback was a “leaky” DNA delivery system. An advantage of lipid coating a nanobowl is that the DNA cargo is protected against nuclease degradation during transport. Moreover, the lipids allow for an increase in complexity of the nanobowl system development with the possibility of further functionalization of the outer surface with polymers or peptides to control endocytosis, specific cell/tissue targeting and opsonization properties in vivo. Although in the present study we did not employ confocal microscopy imaging to ascertain endosome entrapment, we speculate that the lipid-encapsulated nanobowls loaded with clover cDNA were released by endosomes due to clover expression, which we observed with fluorescence imaging (FIGS. 8A-8J, 11A-11F, 14A-14G, 17A-17G) and Western blotting assays (FIGS. 10A-10F). Additionally, the pharmacological assays (FIGS. 17B-17G) revealed that three cDNA constructs were successfully co-expressed and exhibited functional coupling of two opioid receptor subtypes with K⁺ channels.

Our results showed that nanobowls are able to load linearized as well as supercoiled DNA with varying efficiencies. The supercoiled cDNA adsorption reached saturation, while linearized cDNA did not exhibit this property. Such differences in surface adsorption behavior of supercoiled and linearized chromosomal DNA have been previously reported on silica-based clay mineral surfaces. It is possible that once monolayer adsorption is completed for linearized cDNA a different binding mechanism causes the increase in adsorption, such as additional cooperative binding between the free cDNA and the nanobowl-bound cDNA. Another possible reason could be the morphological differences between the compact, supercoiled DNA and that of the dimensionally larger, more rigid linearized cDNA which differ in the density and availability of phosphate groups. For example, linear DNA has a much higher number of available acidic groups along its length than compact supercoiled DNA for multi-loci interactions with clay surfaces. These differences could alter the affinity, the nature of cDNA interactions (electrostatic, bridging, coordination, and/or H-bonding) and protection from nuclease activity in a morphology-dependent manner for silica nanobowl surface adsorption. The presence of the engineered cavity in the silica nanobowls could also affect how DNA (linearized and supercoiled) condenses on the silica nano surface. This may explain the reason for the observed differences in DNA adsorption of nanobowls and Stöber nanoparticles and, ultimately, affect their transfection efficiency. For example, the results shown in FIGS. 7A-7D indicate Stöber nanoparticles exhibited approximately 18% less supercoiled clover cDNA loading and a lower transfection efficiency when compared to equal concentrations of nanobowls in HEK cells. In the present study, we focused on developing a versatile transfection system employing lipid-coated nanobowls. Optimization of Stöber nanoparticles as transfection agents is beyond the scope of this study. Future studies are necessary to examine whether these nanoparticles can be optimized in a similar fashion.

The Western blotting assay (FIGS. 10A-10F) and microscopy imaging results (FIGS. 8A-8J, 12, 15A-15D, 16A-16D) showed that the LNB transfection system we developed could be used to transfect all cell lines tested with either supercoiled or linearized cDNA. It should be mentioned that the clover expression ratios determined from the Western blot assays reflect protein pooled from cells that express proteins either strongly, weakly, or not at all. The clover amounts detected, therefore, do not discriminate between protein pooled from weakly or strongly expressing cells. Overall, our results suggest that LNBs loaded with either linearized or supercoiled cDNA can be employed as transfection agents with the cell lines tested. Thus, the LNB system exhibited versatility in delivering either type of DNA construct with comparable transfection efficiencies.

Previous studies have shown that transfection with appropriately linearized cDNA construct is more likely to become incorporated into the cell's genome resulting in higher success rates of obtaining stably transfected cells when compared to supercoiled cDNA plasm ids. Further, linearized plasm id bacterial resistance genes have also been considered as the method of choice for vaccinations. The disadvantages of employing linearized cDNA for transfection includes the susceptibility to exonuclease digestion, as well as inefficient encapsulation by the lipids used for transfection. Our LNB system showed similar or higher transient transfection efficiencies, including higher DNA condensation capabilities for linearized cDNA constructs as compared to supercoiled plasmids. In addition, we demonstrated that the PCR-based linearization technique allows for incorporation of specific functional groups on the linearized cDNA enabling chemisorption and controlled release of cDNA from surfaces.

Our release studies from nanobowls loaded with linearized cDNA show that the choice of conjugation chemistry is advantageous in order to control the release rate of cDNA. That is, the reducing agent-specific release is more than double for click chemistry than EDC-based conjugation chemistry. This is partly due to the pH of the solution employed. Under our conditions (i.e., pH=7.4) electrostatic-driven physisorption on the amine-functionalized nanobowl surface is even more likely for Lin-C. As a result, both chemisorption and physisorption take place simultaneously during loading of Lin-C, and during release, it is much more likely to re-physisorb back to the amine-coated nanobowl surface. Steric hindrance applied at the nanobowl-buffer interface in the form of biocompatible polymers like PEG can also help in favoring chemisorption and minimizing post-release physisorption. Furthermore, in the present study, the disulfide/azide or disulfide/carboxyl groups were added to only the forward primers for PCR. This leaves room to further tune the nanobowl-DNA release properties by incorporating these chemical groups to the forward as well as the reverse PCR primers during the linearization step.

One major advantage of loading nanobowls with linearized cDNA is that the DNA can be easily functionalized with a variety of terminal chemistries for efficient conjugation to surfaces and release using cleavable bonds built into the primer design. In a biological system, the disulfide group is assumed to be broken by reducing agents such as glutathione (GSH), which is found in the cytoplasm and facilitated the release of DNA from the nanobowls. GSH concentrations are ten-fold higher inside the cellular environment than the extracellular space thereby making the release controllable post-cellular internalization and endosomal release of the LNBs. This becomes a crucial feature in in vivo applications where LNBs are likely to spend a greater time circulating in the plasma/CSF before becoming internalized. The ability to chemisorb and release DNA in a controlled manner also puts the LNB system at a more advantageous position than simple liposome-based systems where DNA complexation and release cannot be controlled with chemical conjugations.

The MTT and flow cytometry assays, which measured LNB toxicity with or without DNA loading, showed that cell viability was greater than 80% at the concentration (0.5 mg/ml) employed. When compared to the commercially available Lipofectamine 2000, the viability measured via flow cytometry was approximately 70%. It should be noted that transfection with Lipofectamine 2000 was not optimized under our transfection conditions (i.e., beyond the focus of the present study) and employed as a positive control for flow cytometry assay (FIGS. 9E-9F). Nevertheless, the toxicity measured for LNBs (92.4%) was lower than for Lipofectamine 2000 (70%) with comparable transfection efficiencies as evaluated with fluorescence microscopy (FIG. 12).

Our results demonstrate that LNBs can be employed to transfect multiple constructs simultaneously, in a relatively fast, inexpensive, and reliable manner in order to determine the pharmacological profile of G-protein coupled receptors, such as opioid receptors. The GPCR subfamily of opioid receptors μ, κ, and δ are clinical targets for a massive number of pharmacological studies especially in the area of newer drug design and understanding mechanisms of desensitization, tolerance, and addiction of highly potent opioids such as fentanyl. The use of high throughput signaling assays like FLIPR© therefore hold unprecedented clinical value in the face of the current opioid crisis.

We have also demonstrated that we can successfully transfect DRG tissues ex vivo with linearized cDNA construct. Our results show transfections in both neurons and glial cells. The nanobowls used in this study have been previously demonstrated to be multifunctional by IONP functionalization and gold coating that gives it the ability to be magnetically guided, localized, and used for IR or MRI based diagnostics. Therefore, this study shows promise of in vivo therapeutic and diagnostic applications of LNBs within a neuronal context.

As the fluorescent images alone provided limited determination of transfection efficiency, we relied on flow cytometry (FIGS. 9E-9F), Western blotting (FIGS. 10A-10F), and functional coupling with the FlexStation 3 reader (FIGS. 17B-17G). One drawback to flow cytometry quantification is that we can only measure clover expression in single dissociated cells, whereas clusters of cells (made up of clover-expressing and non-clover-expressing cells) were excluded. The result is that the percent efficiency is underestimated, with an approximate value of 10%. This value was lower than that obtained with Lipofectamine 2000, which resulted in approximately 15% efficiency of transfecting single cells. The Western blotting technique did allow us to determine clover expression levels and the results demonstrated a very good signal (FIGS. 10A-10F). Finally, the FlexStation 3 results (FIGS. 17B-17G) suggest that pharmacological studies can still be performed with the transfection efficiency we obtained. That is, expression levels for both opioid receptors and GIRK channels were sufficient to obtain functional coupling of G protein coupled receptors with ion channels. The pharmacological parameters determined were comparable to published reports. However, it is also possible that the incubation time post-transfection we employed may not have been sufficient to allow for further clover expression. For example, the TEM images showed that 24 hours post-transfection, there were lipid-coated nanobowls that remained within the vesicles (FIGS. 8E-8F). The endosomolytic properties of the LNBs within cells is another parameter that can be further tuned. For instance, the use of endosomolytic peptides, like H5WYG or incorporation of pH buffering polymers in the outer lipid encapsulation layer of the nanobowls that can lyse endosomes by the proton sponge effect, may serve as an alternative to increase the release of the LNBs that result in greater expression levels.

Our studies showed that these nanobowls are nonspecific and are taken up in cell lines of different origins, neurons, and glia. For more targeted applications, this LNB design needs further development to add target specificity and IONP attachment for magnetic localization. Adding polymers like polyethylene glycol (PEG) in the form of functionalization or physical introduction in the lipid bilayer are needed to improve the stability of the LNBs in protein rich media (beyond the time scales of in vitro transfection) and to further facilitate in vivo transport. LNBs at their current design were unable to cross the meningeal layer in intact DRG tissue enough to cause successful transfection, and we therefore facilitated their tissue uptake by partial dissolution of the meningeal layer with DMSO. Fundamental transport studies of LNBs in connective tissues like the meninges needs further exploration with or without magnetic guidance to be a viable non-viral in vivo transfection agent.

Taken together, we have shown a non-mesoporous silica nanobowl system that can load and deliver both linearized and supercoiled plasm id cDNA. Lipid coating was essential to release nanobowls from endosomal entrapment and can be further optimized/functionalized for faster and more efficient transfection. PCR-based linearization helped attach functional groups for covalent linkage of cDNA to nanobowl surface and reduction-controlled release. We also showed that neurons and glial cells from rat DRGs could be transfected with the LNB system under in vitro or ex vivo conditions. Finally, we have demonstrated the applicability of this system by co-transfecting 3 different cDNA constructs to express opioid receptors, coupled GIRK channels in HEK cells, which resulted in opioid concentration dependent membrane hyperpolarization.

### Example 2. Targeted and Controlled Delivery of SiRNA and Therapeutic Agents Using Nanobowls for COVID-19 Treatment

In this study, the use of nanobowls loaded with siRNAs and therapeutic agents targeting SARS-CoV-2 for targeted delivery and drug release is tested. Targeted delivery of multiple drug molecules can effectively interfere the SARS-CoV-2 infection and related alignments. Targeted delivery will reduce the administered doses of drugs and their side effects. External stimuli mediated controlled release of drug molecules improve the therapeutic effect and treatment. Specifically, the following are tested: (i) magnetic silica nanobowls for delivery of siRNA and multiple drug delivery; (ii) in vitro, lung epithelial cell uptake, and magnetic release; and (iii) inhalation/injection in mice model to determine the pharmacokinetics and toxicity of nanobowls.

There is an urgent need to develop a new strategy to suppress SARS-CoV-2 infection-related morbidity in sick patients. As SARS-CoV-2 is a single-stranded RNA virus, it is possible to select several conserved open reading frames for an siRNA-based therapy. However, development of effective siRNA therapy is limited by poor targeted delivery in vivo. Among viral and non-viral delivery systems, no delivery system is able to deliver to a broad range of cell types with fewer limitations and side effects. For effective treatment, it is essential to deliver multiple drug molecules at the target site. However, the treatment of COVID-19 and related health conditions usually requires high doses of multiple drugs. Such an approach leads to untoward effects at off-target sites. Selective delivery to target sites through a focused, on-demand delivery system would ensure the adequate local concentration and mitigate systemic untoward effects. In this regard, we have developed magnetically guided and stimuli-responsive polymer-gated, multifunctional theragnostic delivery nanocarriers. These nanocarriers allow controlled on-off cargo release in the presence of applied AMF, heat, light, pH, and other biochemical manipulations. Preliminary results show magnetic field-mediated focusing, on-off release and accumulation of drugs in model systems. Loading different drugs in nanocarriers and controlling their release in the presence of stimuli will mitigate the off-target mediated side effect and will facilitate effective treatment of COVID-19 patients.

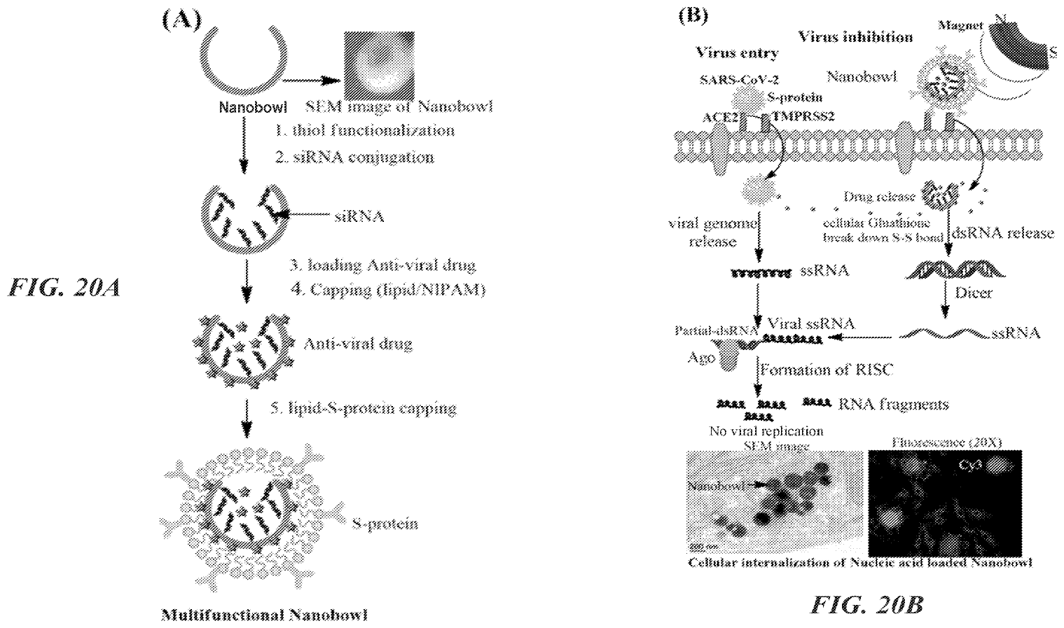

The SARS-CoV-2 pandemic forced health agencies to repurpose drugs such as rem desivir, lopinavir/ritonavir, Interferon beta-1a, and chloroquine/hydroxychloroquine for COVID-19 treatment. Unfortunately, these drugs alone were ineffective in curbing COVID-19 and related ailments, such as pneumonia and inflammation. Here, we propose a new siRNA-based intervention strategy in combination with repurposed drugs for COVID-19 treatment. However, administration of multiple drugs in high doses causes untoward effects at the off-target sites. Controlled targeting and on-demand precision delivery system to predetermined sites (tissues/organs) will solve the problem related to drug side effects and lower the dose (see FIGS. 20A-20B). However, detailed evaluation of nanocarrier safety and efficacy in animal models is needed before consideration for clinical use. In our proposed work, we will: (i) enhance the targeted delivery of repurposed drugs, siRNA, and interfere with virus S-protein and host ACE2 receptor interaction; and (ii) improve the responsiveness to applied DC magnetic field for focused delivery. Development of a focused, on-demand (conditional), and trackable nano-delivery system will allow for evaluation of the therapeutic efficacy of repurpose drugs, as well as siRNA strategy to combat the COVID-19 crisis.

The proposed study will provide novel therapeutic strategy for targeted and control delivery of therapeutic agents for COVID-19 treatment. This study, for the first time, proposes a siRNA-based targeted intervention of viral replication and controlled delivery of multiple therapeutic molecules for the COVID-19 treatment, which will effectively prevent SARS-CoV-2 infection and related health concern.

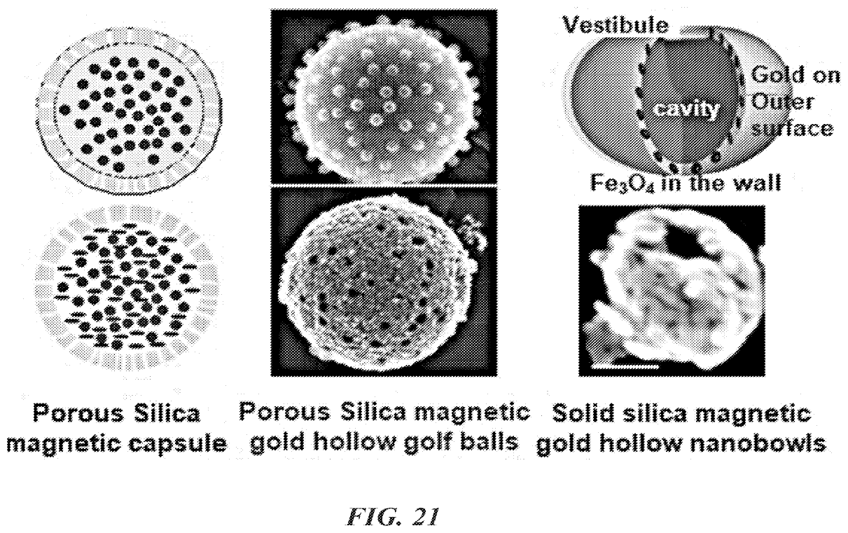

Several nano drug delivery systems have been developed using liposomes, polymers (e.g., chitosan, poly(lactic-co-glycolic acid) (PLGA), inorganic matrices such as iron oxide, gold nanoparticles, mesoporous silica, etc.). Many systems contain functionalized surfaces for attaching antibodies or homing molecules for targeted therapy. However, these systems have inherent limitations which preclude their effective clinical application. Our nano-delivery systems (silica-magnetic capsule, silica-gold magnetic nano golf bowls, and silica-gold magnetic nanobowls) (FIG. 21), have flexible modular design allowing rapid adaptation and integration for specific diagnostic and/or therapeutic applications, making this an ideal platform of technologies. The nanobowls can be porous or non-porous and can contain multi-surface features. The outer surface can be tailor-functionalized for target (cells, tissue) recognition or for capturing and encapsulating external biomolecules. Their inner cavity can be tailored for defined payload capacity which will be unfeasible for currently available nanoparticle-based delivery systems. The gold and iron particles allow on-off release of the payload by RF magnetic heating or NIR-based heating of the nanobowls. Nanobowls, when coated with liposome, allow protection against immune response, spontaneous leakage, and blood shear force.

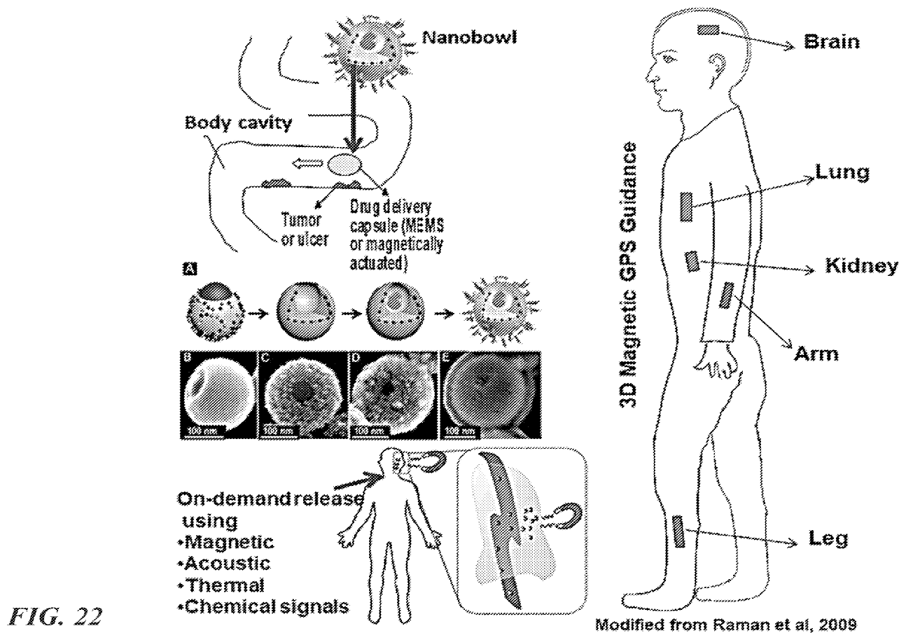

As described above, unlike the other nano-delivery vehicles, the nanobowls of the present technology (FIGS. 21-22) can be designed as a hierarchical, multi-component system with a hollow cavity. The cavity can be functionalized to carry different payload types like hydrophobic/hydrophilic/ionic compounds. The cavity volume is tunable to increase payload capacity. Finally, the entire nanobowl, including the vestibule, can be capped with a heat-sensitive polymer N-isopropylacrylamide (NIPAM) to protect the payload from interacting with the environment, prevent spontaneous leaking as well as a mechanism for conditional delivery in response to a specific temperature.

IO particles are embedded in the nanobowl wall to respond to externally applied magnetic fields. The concentrations of IO particles are tuned to provide higher magnetic sensitivity for a given magnetic field. Since IO particles are buried in the wall, they do not directly contact the biofluids and thus are less toxic. By applying a DC magnetic field, these particles can be vectored and focused to a target site to increase the local bioavailability.

At the target site, when the magnetic field is switched to an alternating RF signal (100-300 kHz), it induces magnetic hyperthermia to activate the heat-sensitive polymer NIPAM. Magnetic nanoparticles (MNP) are heated due to hysteresis loss, and changes in the Neel/Brown relaxation properties affect the polymer permeability to release the drug via the vestibule. Specific response to AC magnetic field prevents any accidental release under DC fields.

According to requirement, the nanobowl outer surface is coated with gold enabling photoacoustic imaging to track the delivery system. Laser-based heating and relaxation during photoacoustic image provides additional functionality to our system, which can be tuned as an alternative strategy for controlled release of a payload for diagnostics or therapeutics.

Since the nanobowl has two surfaces (inner cavity and an external surface), they can be functionalized independently to carry two different types of chemical species. For example, the inner surface could be functionalized with a hydrophobic moiety to carry lipophilic opioids and the external surface with hydrophilic moiety for better stabilization in the physiological environment.

In view of the above qualities, this delivery platform would profoundly impact the management of COVID-19 pathology. In the study, we will test solid magnetic gold nanobowls, as well as porous nanobowls and nano golf balls. The data show: (i) magnetically guided, organ-specific delivery; (ii) pH and heat-mediated on-off cellular delivery of drugs, DNA, and small molecules; and (iii) non-toxicity in animal models.

### Optimization of Magnetic Silica Nanobowls for SiRNA and Multiple Drugs Delivery

First, we will maximize payload capacity for 100-150 nm size nanobowls using suitable functionalization strategies such as disulfide (S—S) based conjugation and cellular enzyme (glutathione) based release of siRNA.

Higher loading capacity would allow a smaller dose of nanobowl, and the focused delivery would further increase the local bioavailability of released therapeutics. We aim to achieve ˜100 nM capacity per nanobowl. The size of the cavity will be increased. siRNA targeting six different regions of the SARS-CoV-2 genome (see Table 4) will be conjugated in the nanobowl cavity. Remdesivir will be loaded in the nanobowl to improve its targeted efficacy. We will use thermo-responsive (NIPAM conjugates) and liposomes (e.g., 1,2-Dipalmitoyl-sn-glycero-3-phosphocholine) for on-off release and conjugate S-protein for targeted internalization.

TABLE 4
SiRNA target sequence in plus strand of coronavirus (MN908947).

| | | | | | | Number |
|---|---|---|---|---|---|---|
| | | | | | | of |
| SEQ | | | | | AR | mutation |
| ID NO: | Target 5′-3′ | Position | Region | Length | (RAR) | strain |
| 1 | AAUAGUUUAAAAAUUACAGAAGA | 6,509-6,531 | Orf1ab | 23 | 20 | 1 |
| | | | | | (20) | |
| 2 | UCCUUCUUUAGAAACUAUACA | 7,168-7,188 | Orf1ab | 21 | 18 | 0 |
| | | | | | (12.6) | |
| 3 | AUGUCAUCCCUACUAUAACUCAAA | 15,041-15,064 | Orf1ab | 24 | 18 | 0 |
| | | | | | (18) | |
| 4 | UUAAAAUAUAAUGAAAAUGGA | 22,391-22,411 | S | 21 | 18 | 0 |
| | | | | | (12.6) | |
| 5 | CUUGAAGCCCCUUUUCUCUAUCUUU | 25,693-25,717 | Orf3a | 25 | 18 | 0 |
| | | | | | (12.6) | |
| 6 | CAACUAUAAAUUAAACACAGA | 27,128-27,148 | M | 21 | 19 | 2 |
| | | | | | (19) | |
| 7 | UUGAAUACACCAAAAGAUCACAUU | 28,688-28,711 | N | 24 | 18 | 0 |
| | | | | | (18) | |

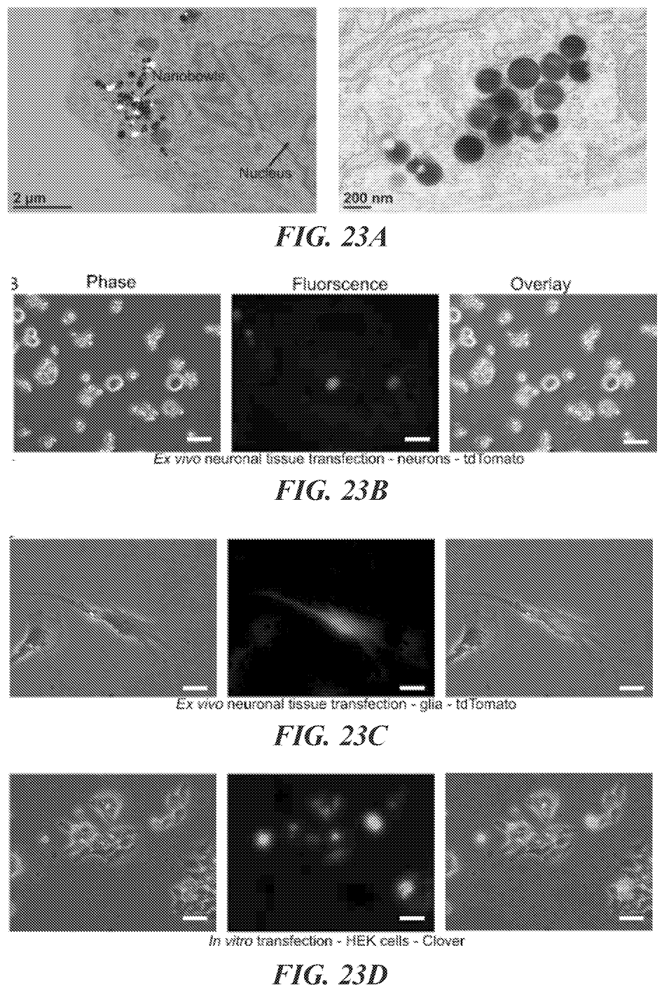

Our current work on pain management via efficient delivery of nucleic acid (e.g., DNA, siRNA) for opioid pain receptors (FIGS. 23A-23D) shows that nanobowls can efficiently deliver nucleic acid (DNA/RNA) intracellularly. We have performed both in vitro and in vivo experiments with Cy3-tagged nanobowl functionalized with lipid molecules. The results show: (i) the nano-delivery system to be non-toxic; (ii) their cellular internalization; and (iii) external magnetic field-mediated delivery of DNA. With further optimization, targeted delivery of siRNA in the lung would prevent SARS-CoV-2 replication.

The inner core of the nanobowl initially contains a PS bead. It is dissolved in dimethylformamide (DMF) to create a cavity. The size of the cavity will be increased by embedding a larger size PS bead. Further, the loading capacity of lipophilic drugs will be improved by enhancing the hydrophobicity of the cavity. We will also utilize thermo-responsive small vesicles (NIPAM conjugates) and liposomes such as 1,2-dipalmitoyl-sn-glycero-3-phosphocholine for surface functionalization. Also, hydrophilic functionalization will be carried out with PEG/charged moieties to enhance hydrophilic drug loading.

Hollow capsules (˜80-150 nm dia), with imbedded magnetic nanoparticles and optional gold nanoparticles, are fabricated using biocompatible SiO₂. The nature of the magnetic materials and the number and size of the embedded magnetic particles will be varied to optimize the effects of these parameters on the cargo delivery and release behaviors. Briefly, PS spheres containing magnetic nanoparticles will be fabricated, followed by attachment of mercapto functional group to a pre-made ˜10 nm Fe₃O₄ nanoparticle layer deposition by chemical reactions. The Fe₃O₄-shell-coated polymer spheres are then treated with DMF or other polymer solvents, such as hexane or toluene, with mechanical stirring to dissolve away the polymer so that only the magnetic particles remain on the nanobowl spheres.

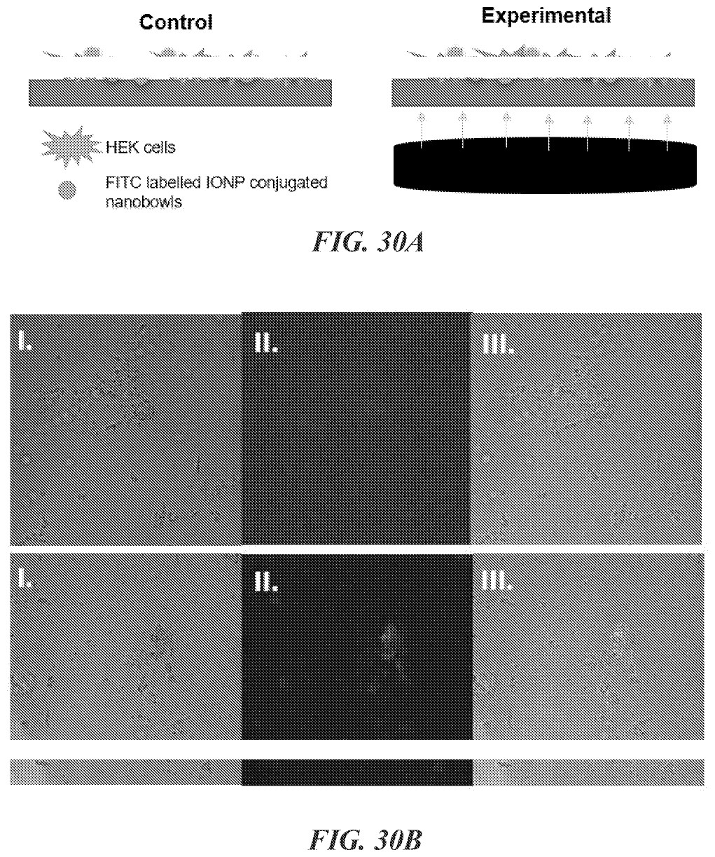

An alternate capsule geometry for drug delivery is to use known biodegradable (or bio-resorbable) materials to coat the nanobowls. These biodegradable materials can be coated by well-known polymer coating techniques, on the surface of these capsules. Biodegradable polymers, such as polylactic-polyglycolic acid (PLGA) or p(MMAco-NIPAM), can be utilized as the shell material. Our preliminary results (FIGS. 24, 30) show successful pH- and temperature-dependent on-off release. We will examine biodegradable polymers so that when all drugs are released, the shell material will bio-degrade, and the magnetic nanoparticles will then be absorbed and metabolically discarded by human body.

For siRNA conjugation, specific target sites of SARS-CoV-2 genome will be selected and siRNA with highest binding affinity will be selected (see Table 4). The siRNA will be custom functionalized to conjugate on the nanobowl. Glutathione enzyme mediated cleavage of S—S functionalized siRNA and its release from nanobowl surface will be performed in in vitro and in vivo conditions.

Next, we will enhance the magnetic sensitivity to applied DC magnetic fields for effective vectorization and focusing in safe magnetic fields (<0.1 T). Currently, the nanobowl magnetic sensitivity is low (with magnetic spins that are randomly oriented, canceling the net moment) thus requiring higher magnetic fields to vector them. Higher magnetic field exposure is linked to several health concerns. We will increase nanobowl sensitivity by optimizing the amount and magnetic orientation of 10 particles so that they require low magnetic fields to focus. The velocity of our prototype nanobowl presently is 0.75 cm/minute or ˜25 μm/second at 0.5 T.

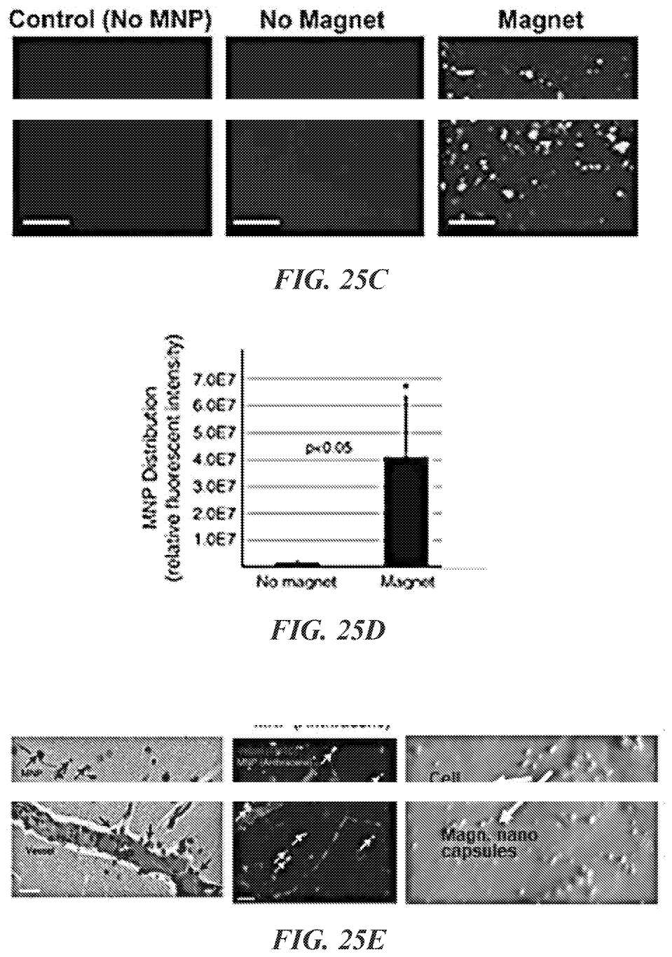

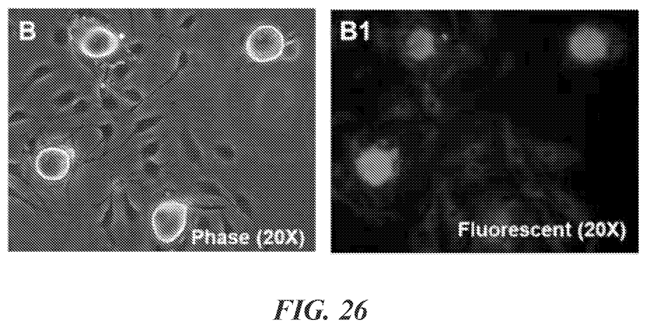

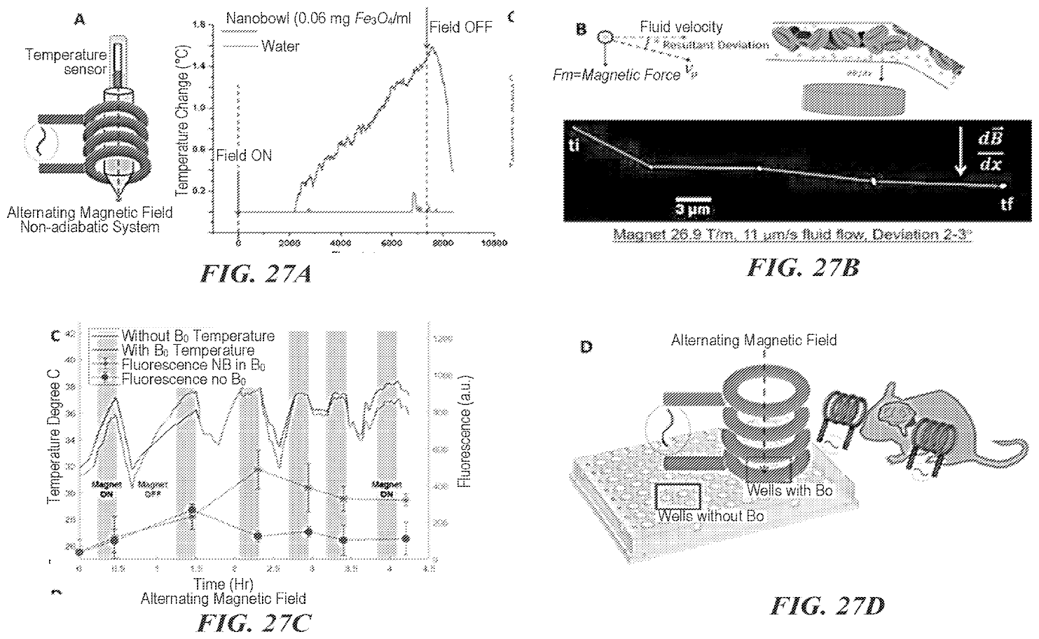

Based on preliminary data, we have successfully delivered magnetically guided porous nanobowls and on/off time-dependent release of anti-cancer drugs in mouse breast tumor model for tumor treatment (FIGS. 24A-24D). We have delivered magnetic porous nanobowls in mice brains using a magnet over each side of the brain (FIGS. 25A-25E). These nanobowls were not toxic in the animals. We have also delivered non-porous MNBs in mice DRG either by direct injection under laser guided system or also by intravenous (IV) injection in mice tail vein. They were held there using a magnet held outside the DRG (FIG. 26). Finally, guiding efficiency for nanobowls was determined in vitro using particle trajectories. Nanobowl cluster trajectories were imaged in different fluid flow and magnetic conditions. VB used was larger than those in commercial MRI machines. In 15 μm/s fluid velocity, clusters of nanobowls deviated 15° due to magnetic force (FIGS. 27A-27D).

To achieve a vector velocity of 1.5 cm/minute, we will align the randomly distributed magnetic moments of 10 particles into unison by the continuous growth of the Fe₃O₄ shell on the nanobowl in the presence of a homogenous magnetic field. Alternately we will also pursue growing Fe₃O₄ in linear mode using salt (Na⁺ and Ca²⁺) based assembly method. Such methods will allow aligning their magnetic moments and avoid canceling net momentum thus increasing their responsiveness to the applied magnetic field.

For external magnetic field guided delivery, several methods will be evaluated for optimal conditions for tissue-specific delivery. Magnetic volume will be increased by increasing the volume of iron oxide on the nanobowl by continuous growth of Fe₃O₄ shell on the nanobowl or by growing Fe₃O₄ in linear mode using salt (Na⁺ and Ca²⁺). Such methods will allow aligning their magnetic moments and avoid canceling net momentum thus increasing their responsiveness to the applied magnetic field. Further, the magnetic volume can be increased for large blood vessels with high velocity by encapsulating several nanobowls in larger flexible polymeric capsules. Flexibility allows for large magnetic volume and compatibility with physiology by preventing obstruction of vesicles. Flexibility of microcapsules will depend on packing density and degree of crosslinking in membrane. Microcapsules of Young's modulus similar to red blood cells (RBCs) can prevent obstruction of microvessels. The microcapsule can be synthesized with materials that have well recorded biodegradable rates so that encapsulated nanobowl carrying the payload are exposed at target site.

The synthesis of flexible nanocapsules will be a three-step process. First, several test MNPs will be encapsulated in a core—vesicle or solid template (silica, alginate). Second, multiple layers of alternating oppositely charged polyelectrolytes (PE) will be grown on the core. PE materials can be such that they are either biodegradable or NIR sensitive biopolymers. The third step is crosslinking of PEs with amide linkages for mechanical and chemical stability. If a solid template is used, an additional etching step will be required. Microcapsules of Young's modulus will be measured on AFM. Their ability to squeeze through small vessels will be tested by imaging their flow through 1-3 μm microfluidic channels.

The nanocapsules' transport activity will be evaluated using microfluidic systems. This would enable determining the efficiency of nanobowl as vectors for targeted guiding of payload. SQUID magnetometry is used to quantify the magnetic moment of 10-embedded nanobowls. Microcapsules' deviation in a magnetic gradient is recorded by brightfield imaging in a microfluidic channel. Guiding efficiency can be measured by flowing through a branching channel while a magnetic field gradient localizes them to one of the branches. Number of particles coming out of each branch is measured by spectroscopy.

Next, we will improve encapsulation to minimize nonspecific drug release and enhance the specificity of conditional release at 37-40° C. The majority of drug delivery systems are leaky and unable to achieve controlled drug delivery. Encapsulation of drug inside solid support and functionalized with external stimuli (e.g., pH and temperature) sensitive molecule such as NIPAM will be an effective strategy to achieve minimum leakage and control delivery.

We will increase transition temperature by magnetic hyperthermia under an AC magnetic field. We will co-polymerize with Poly(N-isopropylacrylamide-co-methacrylic acid) or poly(N-isopropylacrylamide)-co-N,N′-dimethylaminopropylacrylamide to increase its transition temperature above 37° C. and below 40° C. PNIPAAm co-polymerized with N, N′-dimethylaminopropylacrylam ide for capping is sensitive to temperature and pH. Therefore, it will provide a conditional drug release mechanism (On-Off) to regulate drug delivery. To avoid heat-mediated separation of the polymer from the nanobowl surface, we will use carboxylate/amine/thiol functionalization methods and conjugate it to the surface covalently.

For drug insertion and release characteristics, the operational parameters (magnetic field intensity, switch-on/off cycles) for each payload (siRNA and drugs) will be examined. Our preliminary results and published work show unequivocally that payloads, when not loaded in the nanobowls, diffuse in the medium and/or endocytosed by the cells rapidly; when loaded in the nanobowls, they are released slowly. We have been able to load hydrophilic, hydrophobic, and neutral chemicals. We will use these approaches to load repurposed drugs (e.g., remdesivir) and other therapeutic agents following our successful methods (approach section):

We will (i) study the robustness of the nanocapsules that is essential for effective tissue penetration; (ii) investigate the adhesion and internalization in cells and release behavior of nanocapsules; and (iii) study the uniformity of volume, shape, and stability (Zeta potential) of the nanobowls, nanobowls with inserted drugs, and other attached/conjugated imaging and/or guiding molecules size by DLS, EM, AFM.

Finally, we will optimize gold thickness to improve non-invasive imaging such as ultrasound and photoacoustic (PA) imaging contrast from 0.76 to 1.5 for image-guided delivery. Non-invasive imaging techniques such as ultrasound and photoacoustic imaging can be used to monitor the effectiveness of therapeutics. However, these techniques face the challenge of low contrast. Optimization of gold nanoparticles on the nanobowl can be utilized as a contrast agent for photoacoustic imaging. Although, the growth of gold nanoparticles is not the main focus of current work.

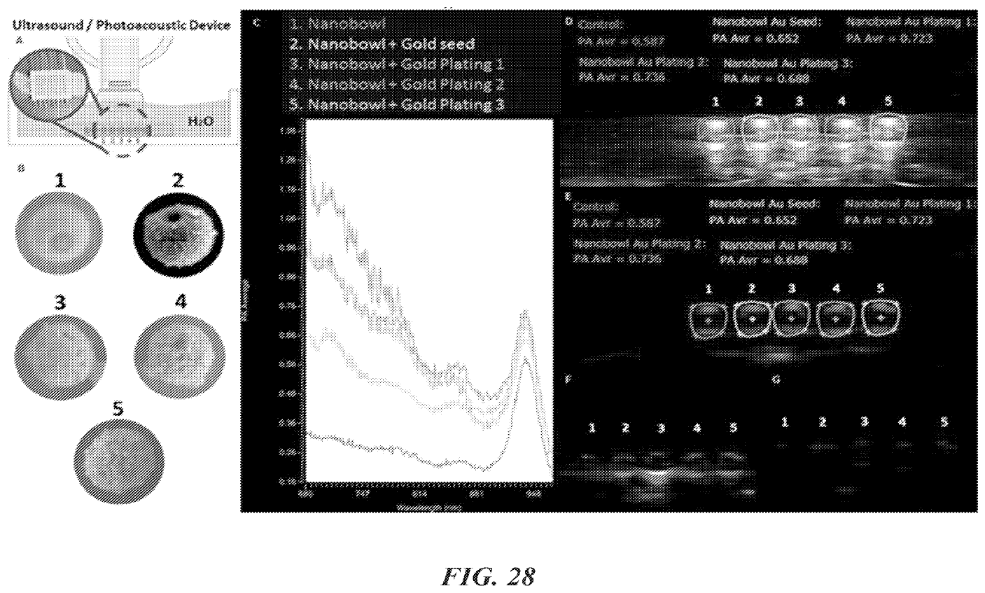

In vitro PA imaging will be obtained by the gold nanoparticle distributed over the nanobowl. We will get a contrast of 0.73, which is barely enough to visualize the nanobowl (FIG. 28). To improve the SNR, we intend to achieve a PA of 1.5.

We will functionalize nanobowl surface to conjugate gold seeds so that they cover the surface uniformly and grow the gold using hydroxylamine hydrochloride mediated reduction of auric chloride solution. Surface gold will provide another modality of the conditional release mechanism. PA mediated heat generation and relaxation can be tuned with wavelength by growing different amounts of gold over the seed to use photothermal drug release mechanism. However, we will focus on the NIPAM mediated on-off release system.

It should be noted that siRNA will be conjugated by S—S bond and its release controlled by glutathione activity. Steric hinderance of enzyme activity on S—S bond due to crowded siRNA may interfere the siRNA release. Therefore, effective siRNA concentration on the nanobowl needs to be optimized in in vitro and in vivo conditions. The insertion of drugs into the nanobowl cavity is limited by the surface tension of the solvent and the trapped air within the nanopores. This can be overcome by ultrasound-mediated vacuum insertion to reduce water surface tension and air entrapment, as well as by hydrophobic cavity functionalization. Moreover, current guided velocity is limited. High F M/F D is needed to achieve the guiding velocity of 1.5 cm/minute. Increasing magnetic volume and minimizing demagnetizing interactions in nanobowls by higher iron oxide/silica ratio in nanobowls can greatly enhance their guiding efficiency for small vessels. Finally, image contrast in the PA imaging is dependent on particle size, shape, and uniformity and thickness of gold. We will optimize the gold nucleation process to get uniform coverage and thickness on the surface.

### Determination of Nanobowl Lung Epithelial Cell Uptake and Magnetic Release In Vitro

Nanobowls will be used for drug delivery, and an appropriate functionalization of the nanobowl will improve its stability, minimize nonspecific interaction, and prolong its systemic circulation. We will analyze the efficacy of our synthesized nanobowl using different cell lines and optimize the functionalization of the nanobowl using different molecular size PEG and optimize its stability under physiological conditions.

We will analyze the multiple drug loading and release profiles and pharmacologic activity of siRNA and remdesivir following temperature-activated release in an in vitro cell model and improve encapsulation to minimize nonspecific siRNA and drug release. We load rem desivir and siRNA onto the nanobowl and analyze the effect of multiple molecules loading in release profile of rem desivir and siRNA, cross reactivity with respect to individual drug loading and release profile in vitro as well as in mice model. The objective is to ensure that the siRNA and remdesivir retain activity in cells and their pharmacological profile following their temperature activated release from encapsulated nanobowls. Functionalization might alter their activity.

Cellular entry of SARS-CoV-2 depends on the receptor binding domain (RBD) of the spike (S) protein and its binding with angiotensin-converting enzyme 2 (ACE2) present in the plasma membrane of cells in the lung, heart, kidney, and intestine. To develop a targeted delivery, it is essential to understand the effect of nanobowl interaction on the cell surface, internalization and release profile of siRNA and remdesivir. We will examine the HEK293_ACE2 and CHO-K1_ACE2 stable cell lines to study the nanobowl-mediated delivery of siRNA and repurpose drugs. Pharmacologic activity of siRNA and drug will be analyzed in presence of external stimuli.

The S-protein functionalized nanobowl, siRNA and drug encapsulated within the nanobowls, will enter using ACE2 enzyme mediated entry pathways. It is possible that the encapsulated siRNA and drug may not exert any effect on the virus. If so, we will optimize the functionalization chemistry to facilitate easy release from the nanobowl once the NIPAM layer opens in response to the specific stimuli.

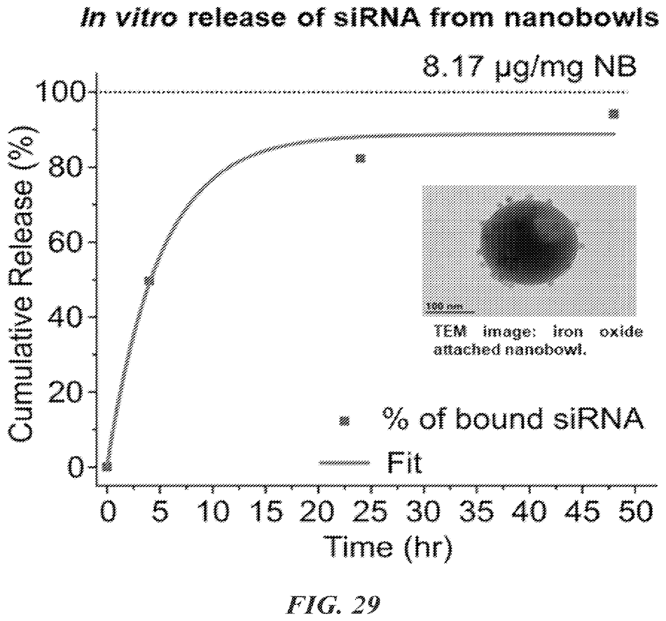

Preliminary data show the loading of siRNA against SARS-CoV-2, release in PS buffer, and uptake by HEK cells which express S2 receptor in the plasma membrane (FIGS. 29-30).

### Determination of Pharmacokinetics, Toxicity, and Efficacy of Nanobowls in a Preclinical Model of COVID-19 Infection in K18-Hace2 Transgenic Mice

First, we will determine pharmacokinetic parameters of injected/inhaled siRNA-nanobowl (SiRNBs) (e.g., tissue distribution, clearance, toxicity) in wild type C57BL6 and K18-hACE2 transgenic mice (COVID-19 mice). In this part, the in vivo characteristics and safety of the nanobowl formulation will be carefully studied to confirm their translational potential. These studies will be conducted using C57BL6 and K18-hACE2 transgenic mice, and various parameters such as circulation half-life and organ-level distribution, will be studied after intravenous or intratracheal administration of the nanobowls. Studying toxicology of a nanoformulation is an essential element of clinical translation, which helps to ensure that the formulation is safe for use in human trials. The following studies have been approved by the University of Arizona, IACUC under protocol (UA Protocol 13-490), University of California, IACUC under protocol S09388 in accordance with local, state, federal, and National Institutes of Health guidelines.

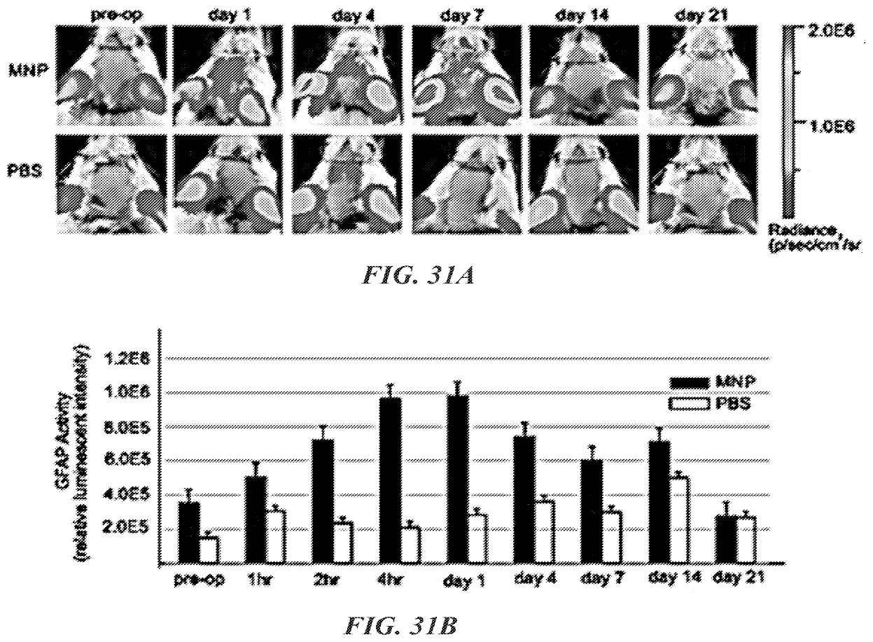

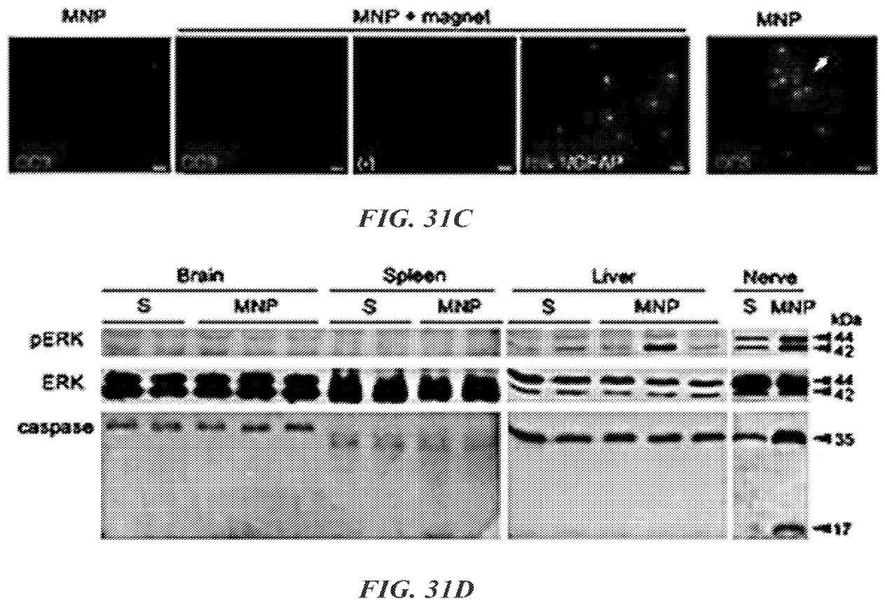

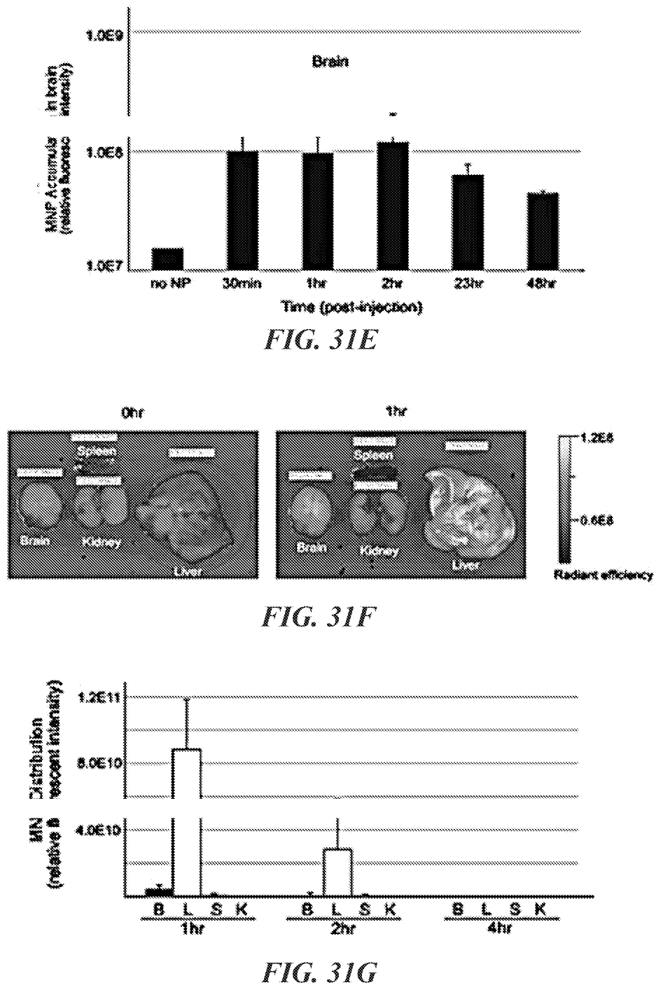

Our earlier work on delivery of porous MNBs in mice models show that MNBs are non-toxic (FIGS. 31A-31G). Our current limited preliminary results also suggest that non-porous MNBs are non-toxic in mice models.

To study the pharmacokinetics of the nanobowls, they will be labeled with the far-red fluorescent dye (e.g., Cy5) to enable in vivo tracking and then administered directly into wild type C57BL6 or K18-hACE2 transgenic mice lungs via intratracheal injection through a 25 g angiocatheter. In separate studies, mice will receive an intravenous injection via the tail vein of wild type C57BL6 and K18-hACE2 transgenic mice. At set timepoints (1 minute, 5 minutes, 30 minutes, 1 hour, 2 hours, 4 hours, 8 hours, 24 hours, 48 hours, and 72 hours) bronchoalveolar fluid and blood will be sampled at sacrifice and the fluorescence (Ex/Em: 649/666 nm) will be measured on a plate reader. Both the absolute half-life and the elimination half-life will be calculated based on a two-compartment model.

To study the biodistribution of the nanobowls, the fluorescently labeled nanobowls will be administered intravenously via the tail vein to wild type C57BL6 and K18-hACE2 transgenic mice. The timepoint for analysis will be informed by the circulation of the nanobowls and will be determined as the time at which <10% of the initial dose remains in the blood. At this timepoint, the mice will be euthanized, and major organs, including the lung, liver, spleen, kidneys, heart, and brain will be dissected and homogenized to determine the fluorescence readings.

To determine the maximum tolerated dose value, mice will be administered with increasing amounts of the nanobowls until toxicity, which will be defined as 10% loss in weight, is observed. If the formulation remains safe, we will establish the maximum feasible dose for a single administration, which will be determined by the maximum concentration that is possible from the manufacturing process. This dose will then be administered daily. Weight measurements will be taken every day for up to 2 weeks after the start of the first administration for these studies. We will also observe for changes in animal behavior (i.e., activity, appetite, fur condition), as well as signs of acute distress, such as trouble breathing or signs of neurological damage.

Based on the maximum tolerated dose, we will further assess safety by performing a number of blood analysis assays. Blood chemistry and cell counts will be performed 1, 3, and 7 days after the first nanobowl administration by collecting blood samples via submandibular puncture. Additionally, histological analysis will be performed on the organs where there is the most accumulation of the nanobowls, as determined by the biodistribution study. Finally, front-line inflammatory markers will help to show signs of acute inflammation, and we will analyze the following, IL-6, IL-1β, and TNFα by ELISA assays.

For all in vivo studies, we will use 6 mice/group, and the ANOVA model will be used to evaluate statistical significance. With this sample size, we aim to have 80% power to detect a ≥25% difference between groups. Microsoft Excel and Graphpad Prism software packages will be used to analyze the data.

Next, we will ensure hemocompatibility through the determination of RBC aggregation and deformability, platelet activation, WBC activation, complement activation, and immunological assays. When a biomaterial comes into contact with blood, different elements in the blood trigger different processes (such as hemolysis, activation of coagulation, protein adsorption, cell, and antibody-mediated immune reaction, etc.) to reject the foreign material from the body. Therefore, it is essential to ensure the hemocompatibility and safety of nanobowls.

Nanobowl and blood cell interaction will be examined by incubating nanobowls with human blood samples for different periods (0, 30, 60 minutes) at 37° C. Unbound blood cells will be removed by centrifugation at 3000×g for 10 minutes and the hemogram of the sample will be analyzed by resuspension of the sample.

Protein adsorption is one of the critical determinants of hemocompatibility. Protein adsorption will be estimated by measuring the amount of protein in the plasma before and after incubating the nanobowl and taking their difference. Total protein in blood plasma will be determined by standard protein quantification assays (e.g., Bradford assay). Samples will be taken at every 2 hours for up to 24 hours at 37° C.

For hemolysis evaluation, 4 ml of fresh ACD (acid citrate dextrose) blood will be diluted with 5 ml of 0.9% saline. Different amounts of nanobowls will be incubated for 60 minutes at 37° C. A control experiment will be performed with saline. All the samples will be centrifuged at 750×g for 5 minutes, and the optical density of supernatant at 545 nm will be measured to assess the hemolysis.

For platelet adhesion evaluation, we will collect platelet-rich plasma (PRP) by centrifugation of the blood sample in PBS containing 3.8% sodium citrate at 1300×g for 10 minutes at 4° C. The PRP will be warmed to 37° C., and nanobowls will be added and incubated for 60 minutes. The unbound platelet will be removed by centrifugation at 1300×g for 10 minutes, and nanobowl will be collected at 3000×g for 10 minutes. Weakly adsorbed platelets will be washed using PBS. The nanobowl samples will be analyzed by SEM. For all these tests, we will continuously optimize the nanobowls to have least interactions with blood elements.

For immunological assays, a battery of tests will be carried out to profile the immunogenicity of the nanobowls in CD1 mice between 12 and 18 weeks of age as it is the animal of choice for immune-toxicological valuations conducted by the National Toxicology Program. Both humoral (lymphocyte proliferation) and cell-mediated (NK cell activity, macrophage activity, and T-cell mediated immunity) and cell viability will be assessed. These assays will be carried out on splenocytes extracted from mice treated with nanobowls for different timepoints.

Next, we will determine nanobowl toxicity in wild type C57BL6 and K18-hACE2 transgenic mice. To examine in vivo toxicity and distribution of nanocapsules, m urine models will be used, and nanobowls with relevant drugs will be tail vein-injected or directly introduced into the lung via intratracheal instillation. DC gradient magnetic field with various field directions will be utilized to induce BBB penetration and targeted attachment of nanocapsules. Remote AC magnetic field on-off switching of K18-hACE2 transgenic mice model drug release will be performed, and biological toxicity, distribution, and disposal of the nanocarriers and released drug will be studied following FDA guideline by histological and histochemical assays, and non-invasive imaging such as MRI, ultrasound, and photoacoustic imaging. We will examine nanocarrier release of the anti-SARS-CoV-2 load in brain tissue mimetics and primary neurons in culture. If and when successful and without toxicity, we will examine delivery and toxicity of nanobowls, as well as the COVID-19 therapeutics in the K18-hACE2 transgenic mice model.

Then, we will determine successful delivery to lung epithelial cells. Our preliminary results including those described above (FIGS. 23A-23D) indicate that our nanocarriers are very well endocytosed by iPSC derived primary neurons (NPC) and DRG cells. The decrease in SARS-CoV-2 load in mice lungs and subsequent recovery will be established by non-invasive imaging, viral particle analysis, and inflammatory marker analysis from lung samples.

Finally, we will examine the efficacy of bixin-containing nanobowls in murine models of acute lung and ventilator-induced lung injury in wild type and K18-hACE2 transgenic mice. The majority of COVID-19-related deaths are due to lethal, inflammatory acute respiratory distress syndrome (ARDS). We will address the efficacy of nanobowl strategies in reducing COVID-19-induced lung injury utilizing a preclinical COVID-19 murine model and a non-COVID-19 LPS/VILI murine model of ARDS. Specifically, we will assess the efficacy of nanobowls encargoed with bixin, a Nrf2 activator and potent antioxidant (PMID: 26729554) in reducing ROS and inflammatory lung injury in the two preclinical models of ARDS (SARS-CoV-2/VILI and LPS/VILI). Nanobowls containing bixin will be delivered one hour after SARS-CoV-2 or LPS infection as a single i.v. administration in each ARDS model. Following a subsequent 24-hour period, in each murine model, mice will be placed on VILI-producing mechanical ventilation (tidal volume of 40 mL/kg, 0 PEEP) for 4 hours, thereby simulating the clinical trial design. COVID/VILI and LPS/VILI studies will be performed at the University of Arizona and will test the nanobowl encargoed with bixin at two concentrations: 2 mg/kg (low dose) or 20 mg/kg (high dose). These studies will directly address whether nanobowls encargoed with bixin can deliver their cargo as a therapeutic strategy in COVID-19- and non-COVID-19-induced ARDS.

We will review proof of concept studies with nanobowls encargoed with bixin in a preclinical “two-hit” LPS/VILI murine model of ARDS. We will use WK18-hACE2 transgenic mice (COVID-19 mice) expressing human ACE2, the receptor used by SARS-CoV-2 to gain entry to cells, with expression driven in epithelia by a human cytokeratin 18 (K18) promoter. We will generate five groups of K18-hACE2 transgenic mice (6 mice/group). Group #1 will be untreated and unchallenged. Group #2 will receive nanobowls encargoed with bixin at highest concentration (20 mg/kg) without LPS infection. Group #3 will receive intratracheal LPS (IT 20 μg, 24 hours) for 18 hours using the K18-hACE2 transgenic mice but without LPS challenge. After 18 hours, we will initiate lung injury with LPS (followed by mechanical ventilation (40 m L/kg, 4 hours) but no therapeutic intervention). Groups #4 and #5 will receive intratracheal LPS (IT 20 μg, 24 hours) for 18 hours and mechanical ventilation (40 mL/kg, 4 hours) but will also have IV delivery of nanobowls encargoed with low bixin dose (Group #4, 2 mg/kg) or high bixin concentrations (Group #5, 20 mg/kg). Mice will all be sacrificed at 28 hours.

We will then review proof of concept studies with nanobowls encargoed with bixin in a preclinical “two-hit” COVID-19/VILI murine model of ARDS. We will again use WK18-hACE2 transgenic mice expressing human ACE2 (COVID TG mice) to assess nanobowls encargoed with bixin in a “two-hit” COVID-19/VILI murine model of ARDS. The Urbani strain of SARS-CoV-2 will be obtained from the Centers for Disease Control, Atlanta, GA, propagated and titered on Vero E6 cells in the UA biosafety level 3 laboratory. The titer of virus used for all studies, as determined by a plaque assay, is 7.6×106 PFU/ml. We will generate five groups of K18-hACE2 transgenic mice (COVID TG mice, 6 mice/group). Mice will be lightly anesthetized with halothane and infected intranasally with the indicated dosage of SARS-CoV-2 in 30 μl of DMEM. Group #1 will be untreated and unchallenged, and Group #2 will be treated with nanobowls encargoed with bixin at highest concentration (20 mg/kg) without SARS-CoV-2 infection. Group #3 will receive SARS-CoV-2 infection and mechanical ventilation (40 mL/kg, 4 hour) as we have previously described but without the nanobowl-bixin intervention. Groups #4 and #5 will be infected with SARS-CoV-2 placed on mechanical ventilation, and receive nanobowl-bixin at either low (2 mg/kg) (Group #4) or high concentrations (20 mg/kg) (Group #5) one hour after virus exposure. Mice will all be sacrificed at 28 hours.

For phenotypic assessment, in all studied groups (non-treated, therapeutic), animals will be sacrificed, and lungs aseptically removed. Tissue culture infective dose (TCID50) will be determined. Phenotypic assessment will include BAL protein, lung tissue albumin, Evans Blue dye leakage, BAL cell counts/cellularity, lung tissue myelo-peroxidase activity, lung histological and immunohistochemical evaluation, and lung and plasma inflammatory cytokines, and the magnitude of ARDS and VILI injury and recovery responses determined by an acute lung injury severity score (ALISS) as we have described. We have developed an ELISA-based meso scale discovery platform (MSD, U-PLEX) to rapidly and accurately measure plasma biomarkers and will measure eNAMPT, Ang2, IL-6, IL-8, MIF, and IL-1RA as we have shown to predict ARDS mortality. Plasma levels in mice will be measured at time of sacrifice. ALISS, will be generated as previously described.

We expect our nanobowls to have multiple desirable parameters: versatility (encapsulating various drug types), low toxicity profiles, drug release modulation; multivalency (ability to bind various ligands due to large surface area); high drug payloads; ability to incorporate, protect, and promote the absorption of otherwise non-orally administrable constructs in vivo. We expect that transgenic mice exposed to LPS or to SARS-CoV-2 will develop pneumonitis similar to human disease, which will be exacerbated by exposure to VILI. We anticipate the bixin-containing nanobowls to significantly and dose-dependently reduce histologic and BAL lung inflammation and reduce circulating plasma biomarkers of injury and inflammation.

After the successful accomplishment of the proposed experiments, we will have a well-optimized and characterized nano-delivery system that has: (i) high drug loading efficiency to maximize bioavailability and minimize toxicity; (ii) high sensitivity to applied magnetic fields (≤0.1 T) for safe and effective focusing; (iii) better drug encapsulation to prevent exposure of payload to environment and nonspecific leakage; (iv) conditional delivery at precise temperature to maximize bioavailability at the target site; (v) established functional activity of released opioids; (vi) better imaging contrast to track these particles at the target site; and (vii) well-characterized pharmacokinetics and toxicity results.

We anticipate a broader impact on COVID-19 patient care by targeted delivery of viral m RNA and rem desivir (nucleotide analog for adenosine), lopinavir/ritonavir (protease inhibitor) to interfere with viral replication, and anti-inflammatory drugs. Also, delivery of imaging contrast molecules and acoustic-optical imaging can be used to monitor efficacy of administered drug and diseases progression.

### Example 3. SiRNA and Repurposed Drug Loading, Release, and In Vitro Delivery with Nanobowls

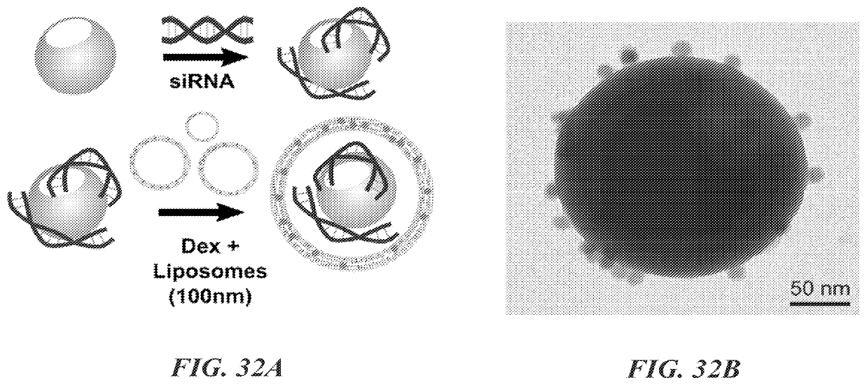

In this study, nanobowls loaded with siRNA and/or dexamethasone, a drug repurposed for the treatment of COVID-19, were tested for their ability in drug loading, release, and delivery into a cell in vitro (FIGS. 32A-32B). As discussed below, these nanobowls contain 10 nanoparticles for thermally activable release of the payload (i.e., siRNA or dexamethasone) when heating the magnetic particles.

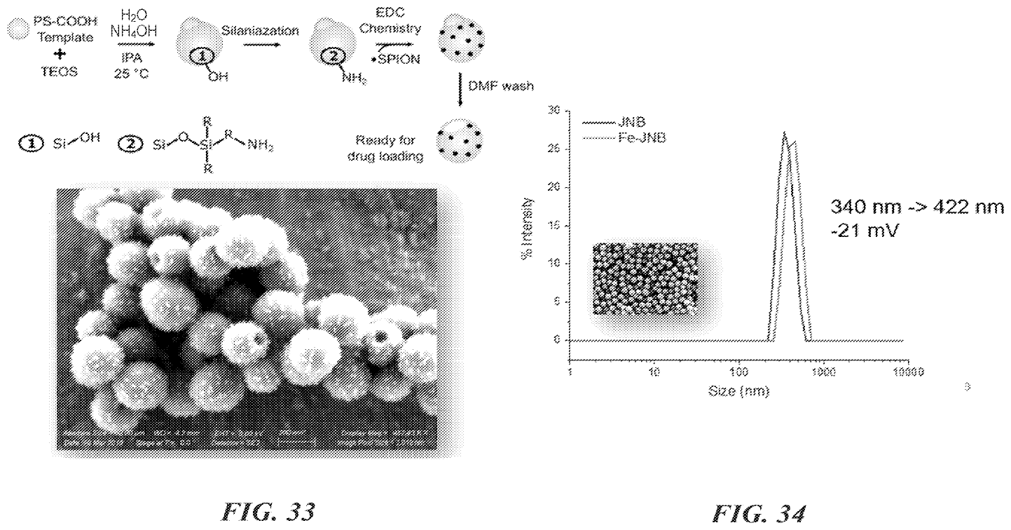

The nanobowl used in this study was synthesized similarly as previously described (i.e., by polymerization of TEOS around a PS template and functionalization with amine groups through silanization (FIG. 33)). Then, the nanobowl was functionalized to be paramagnetic using 1-ethyl-3-(3-dimethylaminopropyl)carbodiimide (EDC) to covalently bind carboxylated superparamagnetic 10 nanoparticles (SPIONs) (10 nm) to the nanobowl surface. The paramagnetic functionalization was essential for magnetic guiding and magnetic hyperthermia for drug release. Finally, dimethyl formamide (DMF) was used to dissolve away the PS template, resulting in a hollow nanobowl ready for drug loading. These synthesized nanobowls containing paramagnetic magnetic nanoparticles are evident by a size change in nanobowl diameter measured using a DLS instrument (FIG. 34).

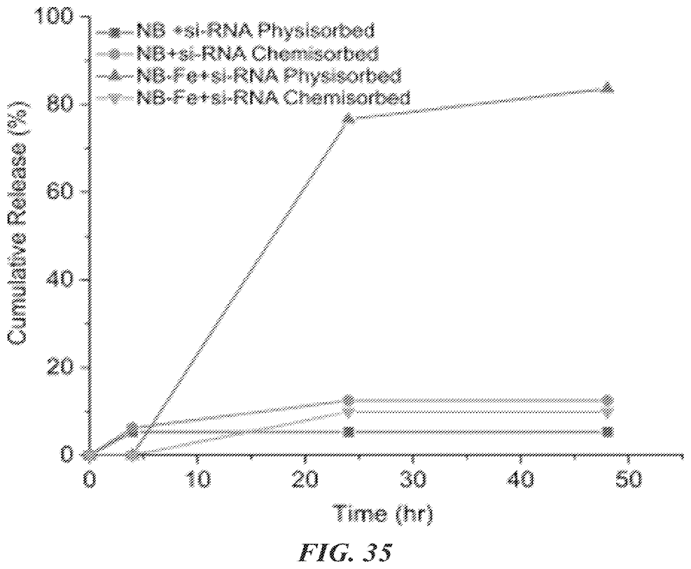

The binding energy of nanobowls to siRNA in their eventual detachment (and release) was first examined in a physiological buffer. This was tested for both nanobowls with paramagnetic iron particles and without. Various reducing agents were used to determine the role of physisorption and chemisorption. Table 5 summarizes different types of nanobowls (with or without paramagnetic iron particles) made by physisorption or chemisorption and the respective amount of loaded/conjugated siRNA. Relative cumulation release after reducing agents is shown in FIG. 35. As shown, it appears that maximal release was achieved when nanobowl is linked to paramagnetic particles by physisorption and then heated.

TABLE 5
SiRNA loading efficiency of nanobowls

| Type of Nanobowl | siRNA Loading (μg/mg nanobowl) |
|---|---|
| Amine (physisorption) | 3.87 |
| COOH (physisorption) | 5.87 |
| COOH (chemisorption) | 5.03 |
| nanobowl-IONP | 2.87 |
| (physisorption) | |
| nanobowl-IONP | 2.03 |
| (chemisorption) | |

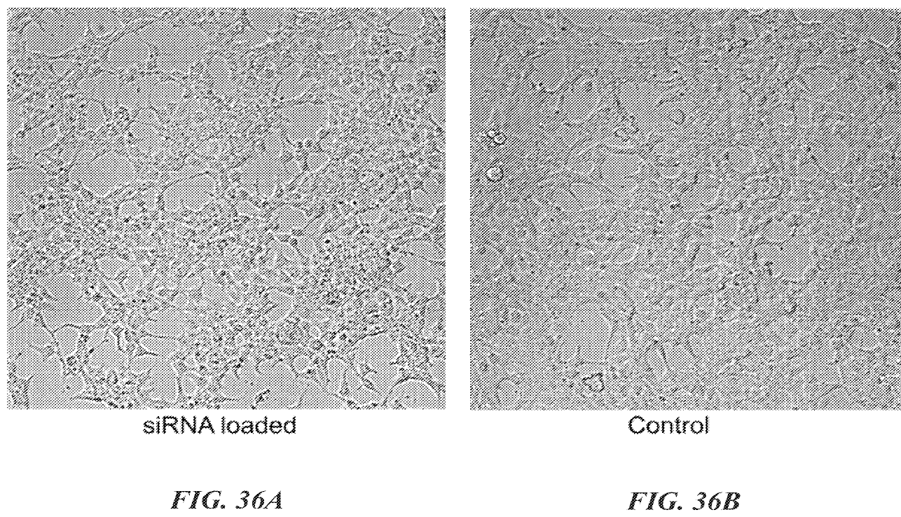

As shown in FIGS. 36A-36B, siRNA uptake by cells in culture were examined using a stable cell line (HEK cells). HEK cells were treated with nanobowls loaded with siRNA or unloaded nanobowls (control). For these experiments, there was no magnetic field applied to force the entry of magnetic nanobowls in the cells. As shown previously (FIGS. 30A-30B), in the presence of a magnetic field, uptake of FITC labeled magnetic nanobowls was observed in HEK cells.

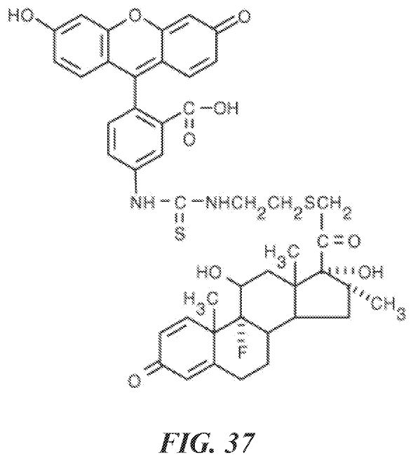

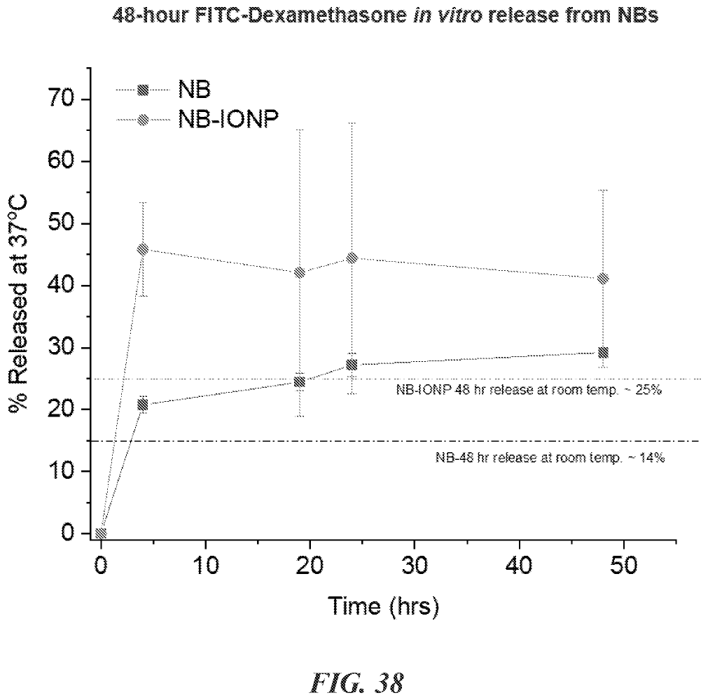

Next, experiments similar to those described for siRNA-nanobowls were also undertaken for nanobowls loaded with the repurposed drug dexamethasone tagged with FITC (structure shown in FIG. 37). Nanobowls were coated with liposomes (1:1 DOTAP:DPPC), and the drug was dissolved into the lipid mixture before coating on the nanobowls. Excess drug was removed by centrifugation post-liposomal encapsulation of nanobowls. Fluorescent emission at 525 nm was used to quantify released dexamethasone. Table 6 summarizes the loading efficiency of dexamethasone (Dex) into liposome coated nanobowls (nanobowl) at different testing conditions. FIG. 38 shows heat-mediated release of dexamethasone from nanobowls with magnetic ion particle coating (nanobowl-IONP, circles) versus those without (nanobowl, squares).

TABLE 6
48-hour FITC-dexamethasone loading
in liposome coated nanobowls

| | Mass of | Mass of | Loading |
|---|---|---|---|
| | Dex (ug) | nanobowl | Efficiency |
| | Added/0.25 mg | Loaded Dex | (% Loaded |
| nanobowl:Dex | nanobowl | (ug/mg) | Dex/nanobowl Mass) |
| 10:1 | 25 | 7.6 | 3.0 |
| 5:1 | 50 | 8.0 | 3.2 |
| 2:1 | 125 | 29.8 | 11.9 |
| 1:1 | 250 | 122.2 | 48.9 |
| 1:2 | 500 | 26.1 | 10.4 |

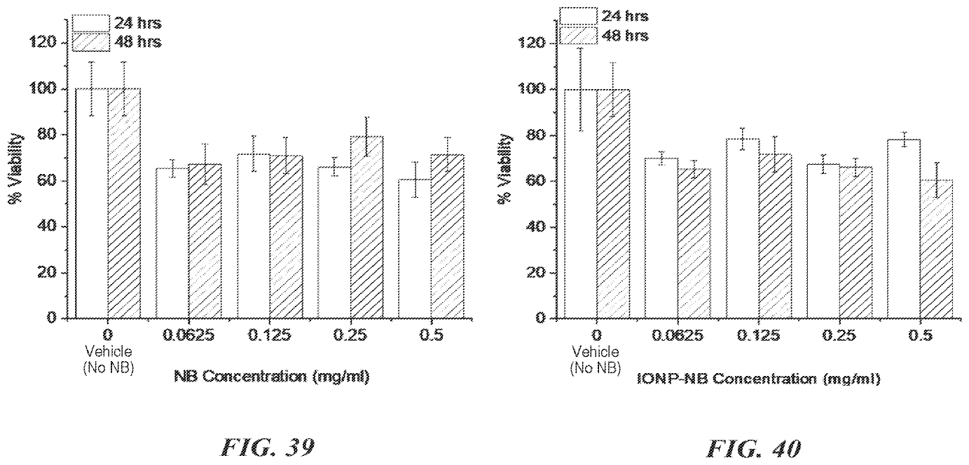

Dexamethasone is known to cause cell toxicity, while siRNA itself has no effect on cell viability. The relative role of siRNA and dexamethasone on cell viability was tested for nanobowls both with and without paramagnetic particles (FIGS. 39-40). Cell viability in response to treatment with siRNA and dexamethasone in a silica nanobowl or magnetic silica nanobowl in HEK cells containing specific cell receptors was shown. When dexamethasone was co-loaded with siRNA, cell viability decreased. However, there is no significant difference between non-magnetic nanobowls and magnetic nanobowls on cell viability.

Taken together, this data suggests that magnetic iron particle coated nanobowls can serve as an effective vehicle for controlled siRNA and/or drug loading and delivery in vitro.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A nanobowl-based therapeutic system comprising a nanobowl and one or more nucleic acids targeting SARS-CoV, MERS-CoV, SARS-CoV-2, or a variant thereof.

2. (canceled)

3. (canceled)

4. The therapeutic system of claim 1, wherein the one or more nucleic acids are conjugated to the nanobowl through disulfide bonds.

5. The therapeutic system of claim 1, wherein the one or more nucleic acids comprise siRNAs.

6. The therapeutic system of claim 4, wherein the siRNAs each comprise a nucleotide sequence that is identical or complementary to a genetic sequence of SARS-CoV-2 that is conserved among different strains of SARS-CoV-2.

7. (canceled)

8. The therapeutic system of claim 6, wherein the genetic sequence is located in the Orf1ab, S, M, or N gene regions.

9. The therapeutic system of claim 6, wherein the siRNAs each comprise a nucleotide sequence that is at least 80%, at least 85%, at least 90%, at least 95%, at least 96%, at least 97%, at least 98%, at least 99%, or 100% identical to any one of SEQ ID NOs: 1-7.

10. The therapeutic system of claim 6 wherein the siRNAs each comprise a nucleotide sequence complementary to a nucleotide sequence that is at least 80%, at least 85%, at least 90%, at least 95%, at least 96%, at least 97%, at least 98%, at least 99%, or 100% identical to any one of SEQ ID NOs: 1-7.

11. The therapeutic system of claim 1, wherein the nanobowl further comprises iron oxide (IO) nanoparticles.

12. The therapeutic system of claim 1, wherein the nanobowl is coated with a heat-sensitive coating, a biodegradable coating, and/or a lipid coating.

13. The therapeutic system of claim 1, further comprising one or more additional therapeutic agents loaded to the nanobowl, wherein the one or more additional therapeutic agents are selected from the group consisting of an antiviral agent, an anti-inflammatory agent, an antimalaria agent, and a biological agent.

14. The therapeutic system of claim 13, wherein the antiviral agent is selected from the group consisting of remdesivir, favipiravir, lopinavir/ritonavir, nitazoxanide, danoprevir, umifenovir, nafamostat, brequinar, merimepodib, molnupiravir, opaganib, and ivermectin.

15. The therapeutic system of claim 13, wherein the anti-inflammatory agent is selected from the group consisting of ruxolitinib, baricitinib, dapagliflozin, eicosapentaenoic acid (EPA), tocilizumab, sarilumab, ravulizumab, losmapimod, pacritinib, bucillamine, tradipitant, lenzilumab, acalabrutinib, otilimab, abivertinib maleate, selinexor, brequinar, ibudilast, apilimod dimesylate, gimsilumab, dociparastat sodium, itolizumab, pemziviptadil, prednisolone, dexamethasone, reparixin, brensocatib, emapalumab, and anakinra.

16. The therapeutic system of claim 13, wherein the antimalaria agent is hydroxychloroquine or chloroquine.

17. The therapeutic system of claim 13, wherein the biologic agent is an antibody specific to SARS-CoV-2 or a vaccine against SARS-CoV-2.

18. (canceled)

19. A method of treating or preventing infections or diseases caused by SARS-CoV, MERS-CoV, SARS-CoV-2, or a variant thereof in a subject in need thereof, comprising administering to the subject a therapeutically effective amount of the therapeutic system of claim 1.

20. (canceled)

21. (canceled)

22. The method of claim 19, further comprising delivering the therapeutic system or the composition to a target cell, tissue, or organ in the subject by application of an external stimulus.

23. The method of claim 22, wherein the external stimulus comprises a magnetic field.

24. The method of claim 19, further comprising controlling load release of the therapeutic system by application of an internal or external stimulus.

25. The method of claim 24, wherein the internal stimulus comprises a biochemical substance.

26. The method of claim 25, wherein the external stimulus comprises a magnetic field, light, heat, or pH.
